SEQUENCE LISTING (23 sequences)

SEQ ID NO: 1 - RNA, 1355 nt, Labrenzia sp. PHM005
atctcttcggagatagtggcagacgggtgagtaacgcgtgggaatatacctttcggtacggaacaacagttggaaacgactgctaataccgtatacgccctatgggggaaagatttatcgccgagggattagcccgcgttagattagctagttggtgaggtaatggctcaccaaggcgacgatctatagctggtctgagaggatgatcagccacactgggactgagacacggcccagactcctacgggaggcagcagtggggaatattggacaatgggggcaaccctgatccagccatgccgcgtgagtgatgaaggccctagggttgtaaagctctttcagcgaggaggataatgacgttactcgcagaagaagccccggctaacttcgtgccagcagccgcggtaatacgaagggggctagcgttgttcggaatcactgggcgtaaagcgcacgtaggcggacttttaagtcaggggtgaaatcccagagctcaactctggaactgcctttgatactggaagtcttgagtccgagagaggtgagtggaactccgagtgtagaggtgaaattcgtagatattcggaagaacaccagtggcgaaggcggctcactggctcggtactgacgctgaggtgcgaaagcgtggggagcaaacaggattagataccctggtagtccacgccgtaaacgatggaagctagttgtcaggcagcatgctgtttggtgacgcagctaacgcattaagcttcccgcctggggagtacggtcgcaagattaaaactcaaaggaattgacgggggcccgcacaagcggtggagcatgtggtttaattcgaagcaacgcgcagaaccttaccagcccttgacatttggtgctacattcggagacggatggttcccttcggggacgccaggacaggtgctgcatggctgtcgtcagctcgtgtcgtgagatgttgggttaagtcccgcaacgagcgcaaccctcgcccttagttgccatcatttagttgggcactctagggggactgccggtgataagccgagaggaaggtggggatgacgtcaagtcctcatggcccttacgggctgggctacacacgtgctacaatggcggtgacagtgggcagcgaactcgcgagagggagctaatctccaaaagccgtctcagttcggattgttctctgcaactcgagagcatgaagttggaatcgctagtaatcgcgtaacagcatgacgcggtgaatacgttcccgggccttgtacacaccgcccgtcacaccatgggagttgggtttacccgaaggcagtgcgctaaccgtaagggggcagctgaccacggtaggctcagcg

SEQ ID NO: 2 - DNA, 68996 bp, Artificial
  Nucleic acid sequence of the Lab biosynthetic gene cluster
ttagactttggatgctgccaatatttcggccagatcccgtaaagtcaccgccttggcaaaactcatcaaagggatggcaatgcccatatcttccatcgacagggtgatgacatccatccgatccaccgaatttgccccgaggtcgaccaggattgactccggttggatcatatccggctcgagttcaggcaacacctcttgcacattgcgtttcacagtctcaaacggatcagtttgactcatgatgttgcgtccctggggttgttcttggcgcaattgaaatcagcggatacgctgtgtgttctacacggatgcagggagagtgtcacgaatgaacaccgcagggattgaagcagttggtgtttatggcggcagtgtttacctggatgtctctgaactggcgcaataccgcggcatggatcttcagcgtttcgagaacctcctcatccgccagaaatcagcggcattgccatatgaagacgcggtgtcgcttggagttaatgccgccaaacccgtgatcgatgcattgtcgcaggccgaacgcgatcagatcgaactgctgattacatgtaccgaatccggtctggattttggcaaatcgctgagcacttatatccatcactatttgggattaagccgcaactgccggctctttgaaatcaaacaggcctgctattccggaaccgcgggctatcagatggcactgaacttcatattgtcgcagacctcaccaggtgcgaaagctttggttgttgcgaccgacttatcccgggtcttggtggacgagaccagtgacgaactgaccatggattgggagtattttgaacccagtggcggggctggcgcggttgcgcttttggtaagcgaccagccgcgcatatttcagtccgacatcggcgccaatggcacatattgttttgaagtcatggatacctgcaggccaatgccagattctgaagccggggactcagacctgtcgctcctgtcctacctcgattgttgtgagcagagctttgctgcttatcgtgcacgtgtcgaaggtgtttcctaccaagacagcttcaactatctggcctttcacacgccctttggcggaatggtgaaaggcgctcatcggcacatgatgcgccggcttttgcgcagtcgtcctgatgagatcgacgtggatttcgaaactcgagtggctcccggattgcgcctgtgccagaggatcggaaacatcatgggggcgactgttctgttgtcactgacaggagccgtgctttatggcgattaccggacgccccagcggatcggttgcttttcctatggctctggctgtgcctcggagttttacagcggagtttctactgctgacgggcagcggcggttacaggacgcgccgattcaaaaagcgctggacctgaggcataaacttaccatgccgcaatacgaggcattgcttgaaggttgcaaggctgttcccttcggcacgcgcaaccaccaaccagatcttgatcaggttccggacatgaaatcctgcattgccgatcaaagcgcccagctcggatatcagcggctcttcctgaaagaaatcaaaaacttccatcgcgaatacgatgtactttgagttgtgttgtctcctctgctccgataggcttacccaaggatacttttaagagcgcttgtctgcgatactggacgttcccatcgcagcaggcgatgtgcgagggaaaatgccattcacgcatttcggcaaatcctaccgaagctctgaggtgttgctgtgactggctgccagagcaaaagagccgggctctcgccgttggcccttttgttgaatgctgcaggccgcgggctttttcctgccgcgggcgtaacatttcgaccggactgccgggccgaagatcttgaagccagtctcgaacctgccgacttcaacattcgaccagccgcggtcgacgacattgatacgctccatatgctggagacagtctgttggccgaaggagctacagacgccgacaaaaaccttggccagtcgggtggcaatcgacccgaatggacaactggtcctcaccttggacggctccccatgcggagtgatatactcccagcggatcaactccgtcgaggctctgacctcttcggatatggacaaggttgacagcctgcgggatccttcaggttcaattctgcatttcctggcaatcaacattctcccaagcgtgcaagaccgtggcctgggcgatgcgctccttgaattcatcctgcactacgccgcacttgctcccggcatcaagtctgccgctgccgttacactttgccgtgacttcacgggacgaaccctatccgatctgaatgagtatttacgccggaagacaccgctgggcacagtggcagacccggtactgcgttttcatgaacttcacggtggtcgtattcaacacccggtaccaaactatcgggcccgcgacacccgcaatctgggcgccggagtgcttgtaacctacgatctgaacaagcgccgcagatctcatgctcctcaaccgcggcaaaaaattgcgcggacggacatcgccaaccgcgtcaattccgcaattcgttccgcgttgggctcaagcagcgatcagttcgaaaaagacacgccactgatctctatgggtttggattcagcggcgatattgggattggcggactgtctgcaagccgagtgcggtagcacactgactgccgcacagcttttcaaacacaacaccgcggaaaaaattatcgcttttctgcacaacgaactgccgtcctccggtttgtcaaagcctacgctgctaccggcgcaaacgagttgccccgcagatggcggttcagaccaaagcgttgccatcatcggcgtctctttgcgcatgcctggcgggatcgaaactcctcaagcactttgggaacttcttgacctaggcggcaccgtcatcactccagtcccttctgatcgctggtcctggccggatggctttcggccgcagggagccgcctatggtggcttcttgcaggatcctgcccgatttgacgccgcattcttccgcatttcaccacacgaagccgaagccatggatcctcagcaaaggatattgctggaattggcctggcacggtctggaggacgcgggcctttccgcgaccaagttggctggctcttccaccggcgtgtttgtcggtgccagcggatcagattatcaacgcgccatggacgctgcgggagtgccggttcaaccgcatcacagcaccggcgcagccttgtcggtgatagcaaaccggctctcatatgcgctggatttcacagggccaagcctggttgttgacaccgcctgttccagttcactggtcgcagtgcatcaggctgtggcagcgcttcaagagcggacttgcggcctggcattggcggcagggatcaatctgatcctgcatccggcaacatcgcaggcttatcaatcggcgggcatgctgtcaccatccgggttatgccgaagtttcggttctggggccgatggttatgtccgcagcgaaggtgctgttcttttagtccttaagcctttggctcaagccctggccgaaggctgccgggtgcacgcggtaatccgcggaagcgcctgtaatcatggtggcatgaccagtgggttgacggtcccgagtccggacaagcaaacggagctcttgtccgcagcctggcataatgcggatataaaacccgctgaccttgattatcttgaagcccatgggaccggcaccaaacttggtgatccaatcgagatagagggcatgaaaacggcgctggctgagttcgatgatagtcagccgaacccccctgaacaacacgcttgcttgacgggttcggtcaagtcgaatttgggtcatctagaagctgcagcggggctggctgggctgtgcaaagtaatgttggcgttacgccatgaacggctgcctgcttcgctgaatgcatccccacaaaatccggaaatctcgctgaacggctccaatctggccatcgctgacaccgctcgagattggccaaaaggaaaccggcccagaatctccggcgtcagcagttttgggtctggcggtacaaatgctcatattgttgtagccgaaccgccggatgccccggatggcgtcatcgatacgggaccgcaactttttgtcctttccgcaaacacgcccgaacggctgatggcgttggcggtacattggcaagagtggttgaagaagcagccgcacgatctgaacatccctgccctttgtcatgccagccgccaccggcgtgccgccttgcctgcgcgctttgcgacaaaagtctcttcacgggcagacctggaaaaagcgcttcaccaagccgctcagaaaaatcccgcatctagtcaggccaaacccaagtttctggaacatctgaaaggagacgctggacaagccttcttgcaggccttggcaaaagagggggacctgtccgccctggcagatctctggtgtgccggggttccggttgattggtcactgattgattcgacgcccccagaacagccggtgccctggattgatttgccattgtatccattcgataaaactcgcttctgggctttgggaaaagcaccggctgttccgcaggatcgggctgcggcaactgcagaactgtacgctccggtctggcaagaactggccgcgagcaaaacgcagatgccagagccagacttgctgtctgggccgtttgcacttaaagccgcgcagcttttaaagctcgatccatcggaaagccggaactcagaaacaaacgccataggcgagaacatgcacgttctctggagcagtgccccgcggcccagcgattccggtgaaacattagaggaattccgggagtttcaggacttcgttgccggcttgcctcgccagttgtcgcgtttgcggctaaccgtggtgacttggaacggacaggccgtgtacggcaacgagccggttgatgccgaggccgccgcgatctcggcgtttacgcatgtcttggcccaggaaaaacccgaatgggacatacgcacgtttgacttggactcgtgtgacccgccctcatggtccagtctcgctgagagcaatgaaacgaggtctgctgtccgggccggtaaagcctatggtttgcggctggccatggccgacccacttccggataccggccaatcgcacctgcgcgaagacggtgtttacgttgtcatcggcggggcgggggcattggcacgacctggagtgaagcggttctaaacaacgtccaggcgcaagttatttggataggccgccgtccacataatgcggcgattacggcacatatcgaccggctgaccaggctgggcccacctccgatctacattcaggcggacgccacgaaccccgacgcccttgaaagggctttgcaagaaattctgaagcgttggggacgaatagatggcgtgattcatgcgatcacaggcccatccgaccagcccatcttggacagtgagccggaaaatctaacccgtgtcatggcagccaaaacccatggtttgatccaaaccgcccacacgtttgccgccttggacctggatttctttttagtcttttcatcgattatttcgctggaacagcccggcggtttcggaggttacgcggccagctgcgcattcgcggatgctttcgttcgcggactggactcccagacaccttaccctgtccggtgcttaaactgggggcattgggatgtcggtgtcgcccgcaatctgcctgaggcgacaaagatacggctggacaacgccggagttgtcccgatcacggctcaggacgcgttgaagcattgcgatacggcactgaatgctccgctgcctcaactggcaatattgaaatggaatgatcctgcccggcatcccctggtcgacagccaggttcatatgcgcctttcgcggaaggcaccggcgcgcagtctcccggctgcaacaaatgaattgaacacacggctgcaggaaatcgagcggcacggactttttgcccatccggagttggaggcggcattgcccggcgcaatagccgcggaacttgaccgccatggcctgcggacatccttgcctgacacggctccgtggtatctgcgccgatggcacaaggcgacgaaacggctccttgcgcaagggaacaccggcgagaactgggatgcgaccgcacgccgtctgcgcgcggatgcggatctggctcctgcgatcaatttggtgacggcctgcctggcacgactgcacgaagtcctgacaggtcagacaccggccactgatgtcctgtttcccggtgcatctctcgatctgctagagccggtttatcgcggcactgcttccgcggatctgctcaacgatgttttggccgatacattggctgaaacgctccgagcagacctgagggaccagcctgagaacacatccttacgggtccttgagatcggcgcgggaacaggcggcacgaccgcgcgggttctgccctgcttgtccgagcttgctggacagattgagacctatgattacacagatctgtcacgtgcatttttgcagcatgcccaacaggcttttgccccaagtgcacccttcctgaaatcactcagatttgacgttgaaaaaagcccggaaagtcaaggcctgcaacccggcagctacgatgccgttctggcaacaaatgtgctccatgccacgccggacatccgccagacattgcgccatacacacgctttgctcaaacctggcggggtgttgcttctcaatgagattgtgaccccgtcagtctttgctcatgcaacctttgggctgttggaaggatggtggaagtcatgcgatccgggcctccgccatcctgacacgccccttctatcagccgagagttgggaaaaactgctgctggcaaacggctttaccgctgttgaaatgcttttgaacagcagcactgcgcttggtcaacaagtctttgctgcccgcagcgacggctgtttcgagtaccggaaggcagagattgacacaacccgcagacaacctgagacgctcgagccgcgcatcctcaagaacacggtcagcgagttgccattggaggacctggaaaatccgcaagctgcggctgcaaggcttttaacagaaatcgtcgctagcgccttacagattacagaagaccagctggatccatggacacctttgggcgactacggattggattcgatcctgaatgcccaggtcaccgcaagattgcgggagctggttccagatctcgataccaccttcctctaccaataccagaccatcgcagatctctcgcaagcacttgttcaaaaacatccagaagcgtttgagcagatcggccacaccacttgcggagaagcggacgtggcatcgccttcgacagtatccgccagcaaaagaaccgcggggaacgaacagcaggacattgctattgtcggcatgagtttccgttttccaaaggctgatacacctgaggaattctggaccctcttgtcacaagggcaaagtgcagtgacggaaattcctcccgatcgctggcaactggacggtttttatgaatctgatccagacaaggccgtagacggctggaaaagctacagcaaatggggtgcatttctggagcgggtgacagccttcgacccgctctttttcgggatcaacccaaaagaagccgctgccatcgacccgcaggaacgcctgtttctgcagaccgcatgggcggcactggaagatgctggatttccgcgccagcgcctggcagatgaactggcacggagtgtcggtgtgtttgtcggtatcacgcgaaccggatttgacctttttggccccgatttgtggcaggcaggtcaaaaggtctatccgcacacttccttcagttcagctgctaaccgcctgtcctggttcctggatgccgatggccccagcatgccggtcgatacaatgtgttcgtcttccctcacagcgctccatcaggcctgtgccagcctcaagacgggcgaatgcagactggcgattgcaggcggagtaaacctctttctgcatccgacaagttacatcgggctctcggcgatgcgcatgttgtctccagatggacgctgcagcagtttcggtgccggaggaaacggatttgttcctggtgaaggcgtagctgccctggtgcttcggcctctggccgaggcccaagccgcgggcgatcaggttattggtgtgatccgaggcagcgcagtcaatcatggcgggcgcacaaatggtttcaccgttcccaatccccgcgcccagagcagtctggtgcgtgaggcgatgtcccgtgcagggcttgagcctggacagatcagctatcttgaggcgcatggcacaggcaccgaaatgggggacccgatcgaaataaccgggttgaccgaagcatttgccgggcgggagcaaggtttggcgccgtgcgccatcggctcgatcaagaccaacattggacatcttgaggcaactgccggattggctggcgtgatcaaggtgctgttgcagatgcgccatcgccagatcgttccgagcctgcacagcagctctctcaatccaaagattgattttgagcatgcgccatttcgcgtcgcgcaggacctcactccatggtccccagctaaagggcgccggatagccggagtttcatcatttggcgccggcggaacaaatgcgcacgtcatccttgaagaagcgccggacatacctgaaaaaagtgcaactgatcccgcgccaaacgaaccgatcgcgcttgtcctttctgctcatgacgaaccgcgtttacgggcctatgcagcgcggctcgccaagttcttgacttcccccaacgcccctcccctggcactggccgctcaaagcctgcaactgggacgagagccgatgcgccatcgcatggctgctgtcgtgtccgataaggctcaggccgtggcagtcttgcaagccgtcgccgagaaccggccgttgcctgacaaaaccttcttgcgggatacacgcaggtacaaggggcaatgtccttcttcggtggaaagtgaagaccttggtgaactgacagatgcatggagcaaaggcagcaaaatcgattgggctaagctccaccaacgccgccaaaccgtatcactgcctacctacccatttgatgaaaaaccttactggttcgccgacaccgcgcctgttgggggacccatggacgtcccctcctctgaagacgcttttagggaattaaaaccggcttctcggccttcaccggtccggcggacactgccaaggctggatactgcaccggcacagtttgagccgcatcgccgcagccaaaagcttcggctgtcttctctgaacccagcgagtgaaacaccgcctgctgaaatcgaattggacatcaacggcatcggcagagttcgcctagagcctgccagcccgccgccaaacctttcaaccggaaacgccatgaaggttctggtggtcgaggggcttcagcattggaacggagaccggttggggctgctgcatgagctcgaccaactctcgcaaccagtaatcctgacagtgtccgcgagttcgttacccccgatcccggatacgcttcttaccgctccagcctttgagcaggcacaggaaatggcaaacgccaccgcacgctgtccggctgccacgctggccaccttaaaaaaccatattcgcaatcaacctagctggccggatatcgcagggattccggcggaatggatggccggcagcggatggccggtttcgtcgcccgagccggcaccttctggcggcgctattccgcttcaatccgaagtcgtccaattgcacgacatggggggcggtgtcgcgcaaatcacaatggccgagcgcgatgcgcaaaacacctttacgcccgcttttgtcactggagttctggaagcgttcgacaaggtcgagtcctctgccgccttcaaggttgtcgttttgacaggctatgaagcctattttgcttgcggtggtacgcgcgaagggctcctggcgatccagaatggacaagcccgctttaccgatgagcaaagctacgcccgtccgctgcgctgtccgattcctgttattgcggccatgcaggggcacggtatcggtgctggctgggccatggggctttactgcgatttggcgatttacagcgaggaaagctgctatcaaagcccctatatgctttatggcttcacccctggagcgggtgcaacaacccttttccccgcgcggttggggcggcaacttgccaatgaaatactattcactgctcagtcattcccaggccacatcctggcacagaagggattgactgcaccggttctaccgcgtgaagaggttttaccccaggctcatgcattggctcgaagcattgcgcaaaacccgcgcgagacgctgatggcccgcaaatccacgcagacagccgaatttctccacatgttgcccaggctgtttgaagcggaactggctctacatgaaagcacctttgtagggaattctgacgttctggagcagataagtgagcattttgccgacaaacagatgacccaaaagcctggcgcatcccagaaagaggcgcggaacacgtccgcgctcaagacgcaactgcgcatgatgcttgcagaggaactggacatccctcctgaccggatagacgacgacacgcctttcgtggatctcggtttggagtccattgcagctgtcatctgggttcggaaaatcggcgaagagctcggagcccagatcggagcaaccagtgtctatagccaccccaacctggcagcatttacagaactggtagctgagaaaggtggccagctggccgaggcggtcaacaagaccacagcacccccttccgagcccccaaaagccgccatccctgccgatccggaagagcgccttttgccgtcagacagctctgatctttttgtctggctgcaggcatctttggaaacagagctctccatcccatccgggacgcttgatcctgatcgcccgttcgtggaactcgggctcgattcggtgactgcagtcacctggatacgccaggtcaatgacgccctgggcaccaaagaaactgggaccgtggtctatcaccacaccaacctgactgaattggcggcctatctggcgggcattgccggcaaaacacctactaccaggaccacttccttaccatacaagctggaggcaccagtacgatccgccttgcctcggctggaaaatctagcgcctttccaagatgaaagacccggaattgcgattgtcggtatggcgggccgttttcccgaagcgcccaacgtgtccagcttctggcagaatgtcctggctggccgggattgtgtctatgagattcccgccacacgctggtcaatcgacgcctactatgatccggaccgccaggctccaggcaaaaccgtttgccgcagaatgggtgcgattgaagacatcgacgcattcgactctctgttttttggcatttcgccagctgaagccgagctgatggacccgcaacagagactgttcctggaaaccgcctgggaagcgatagaggatgcgggacacgcgccgtctaccttagccgggacacgatgcggtctgttcgtcggcactgaaaacggagactatgcccggattgccggtgatgccaaacctgaagcattggcgctgaccgggcgctccgtggcgatgctcccggcgcgtgccgcctatgcattggatctacagggcccctgccttgccattgacacagcttgttcggcgtctctcgtggcaattgcccaagcctgtgccagtcttcacgaccgtcactgcgatagcgcgctcgctggcggtgtaaatgttctgaccggtccggaaatccatgtcgcgatgagccatgccggcatgctgtccccaagcggcaaatgcaacagctttgacagccgcgcggatggttttgtgcccggagaaggcgttggcgcgctccttttaaaacggttggaggatgcacaggccaacggcgacgatgtttacgcggttatccggggctggggggtcaatcaggacgggcggacgaatggtatcactgctcccaaccccgcagcgcaaactcgtttacaaacagagctttaccaccggttccatatcgatccggctcggatcggcatggttgaggcgcatggaaccggcacggctcttggcgatccgatcgaagttgaagcactcaagcgaagttttgctcagttcactgaccgcaagaattattgcgcgctcgggtctgtcaaaagcaacatcggtcacttggccacagccgcaggggtcgccggcgcaatcaaggcaacactagcgttaaagcaccgcaagatcccagccagcattcatcatgatcagctgaacccgcatatcgacctcaaagacgcgcctttttatgttccgcggactgcagcggattggacagctggtccggacgctccacagtatgcggcagtgagttccttcggatacagcggaactaatgcacatttggttctggaagcggcaccggcaagacctgttccggttacgcagacccaagcagtgattgttccggtttcagcccgttcattggaatgcttaaccgaagccgtgacacgattgtccacctatctgggaaccggtgccggacagactgtccccttggcagatcttgctctcacctatcagactggccgggatacctttgaccagcgtgtagcgttccttgccgacagccacgacagcctccgagcaggccttgaacagttcttaaacgagcctgagcatgctggcggtgtcgtctactcaaatgacatgccaccgacacttcgtgataccgccacggcctggatcgaaggcaagacaatcgcgtggcctgtggtagctggagcaagccggcggcacgggtgtccgacctatccgtttgccaaggagcgccattgggtttccgatgcgcccgtggaattgccggaagctgcacccataccctccaaagagacgcccctccaaccggaagccgaagacacagctgttgatcccgattggcgtgaacgcttaaaacagcgttttgcccgaccaattacactgttgtctgacgatccgaagtggatcgggtccatggcatccctgctgtccgcgcttggcgctgctccgggcggaccgggacagccggacctgcgcatcaaatccaatctgcgtgaggcggaggggagcgttttctgcgacacacatctcggaacacggttgcctggaaacgaacaagtggatttgttaatcctgacagaacttccttcggacccgggcctgattccacagcatgcgctgattgttagcgacgataaccgggatgatatcgaatcccactgccagcgattgatccaggaatggctccgattggagccggacggctcaaaagataccctgcacgtacaattccgaaacgggcgccgtttagtagcggcgaagcctctagatccggctgacggtgcttgcatcttgcgaaagacatggcagcgcacgcctttggctgaccagaaaaccgctccatcagacaaaaacgtctgcttgatcggccgtggccccaaattcgaggcgctggcttctggtcttgaggcccactttcagtcagtcactttacgggacactccgccggaaggggcgatggcggcgtgggatgtgtttatcgacgccgccgctctgactgaagtgagagacaacgatccggacgaccctgaccgcagacactggatccaatccctcatgcgtgagggccgggacctgaacttgctgcacttgacgtgtgatgtgataccgttccgcagtgtttcccgcaatctggccggggcgcggcaagccgggttggtcaagaacctgcgcgccgaataccggtttgcagagtcccggtggctcgatctggatatggcgcaggtcgcagatacagctggcctggcgaaactcattgcggccgaatgtgcgtcagccggaccggtctccgaggtttgttatcgcggcggcgcgcggtttgcgccggtacttgaggcacctgagccggtcgcatcaccgtccgttcacctgaacgcggaaggactgtatctcataagcggtggcacccgcggcgtcggtttgactttggcgcaggacctggcagcccagggagcccgacatctggcgctgattggtgaaacgcctttgccgccgatgcaggactggcccagtctgatcgccgcggctgacacgcctgctgaaatccgcagtcaattgagcatcttgcaggcattgtcagatcaattggaaactctggaaatcttgcatgcctgcgtcagcgatgcggccaaagtgtctgcatggctctcaagtctccgcaaacgcggcctgccgctcagcggcgtgatccatgcagccgggcgctattctgaggtagacccacccggttttgccgccaagtctgccgatcacatgcgcgccgtactcacagccaaggcagatgggctggagaccctccatagtcttacgaaaaacgacccgctttcttttcttcttgtgctgacttcaataaccggcttggttccacacttcgcacgaggcgccctggattacgccatggccaatgcttatgcggatctttttgctgccaaagcccatgaactggatggtggacgcacccggtcgacaattctcagtgactggacgcaaagtggtgcgttctgccgtgtcagaccagagaaagccaagtcggtccaaaagaatttcgatcaaattggattaaagaccttgagtgatgctgaaggctgcgcccttatccggcgggcgctgtctcccactgcggagaccggcacaatcttgggtctgatcgcggaagaccggtttgctgctgcccgcccgggcctgctgctggccggaacgttaaacgatgaggccttggacatgaatacccagcttgcacgctgggaaaaaatccgctcccgcggggatcttgtaaccattgaagacgtcacatctgtaatcggcctggaacagatccgtgaattgcccccgcgcaaatgcttcgcctccaccggatcatgcttggccccactgaagtagttcctcccgaagctgaggatgagtctctgccggacatgatcgccgggattgtctgcaacgtgcttaaactcaaggagatcgaccacaatacgccgttacagaactacggcctcgattccatctcgggcatgatactgagcactcggctggaaatagctttagacatgacggtcgatccgcgcacattaatcgatcatccaagcatcgccgccttatcagcctatatccaaaaagcacgggaagcggcatgagccagagcatagaggaacttttaggagtcgataccttaccgaagccgtccaggcggcaaaacatgcgatttagctgcctgttcttttccgatgtgcgcacagacatctcatatgccgagaagtaccggtttcttggtgatgtcacccggttcgccgatcaaacgggtttcgaagcggtttatttcccggaacgccatttccacgaattcggttcggtctttgccaatcccgcaatcgccgcagcgcatctcattccccaaacacaaaacatccgctttcgtaccgctggtgtcaccatcccgctacaccatccagcggagattgtggaatggtgggcgatgaacgatgttctatcgggcggacgggtggatcttggctttggctcaggttgggccaagggagatttcatctatgctccagaaaactttgaagatcgccgcaaaatctgcagcgacggcatagagacaatcaaacgtttgtggcggggcgagacgctcgcctttcccggacccgggggcgatgttgtcgacatcaccgtctacccccgtccaatccagtccgatctggcggtctggttgctgataactcagaacgaagacgccttcatccacgccggaaagatgggctacaacgtgttcactatgctctatgggaccaacctggagaacttgtcccaaaagatcgccttgtatcgcaaggctcggcaggaggcgggccatgatccggtcagcggcagagtaaccctcacgcttcataccctgctgctcgacaccatggactcagttctggcagccatcgaagtcccattccgccagtacatccaaagcagcctgaacgcccacgtgaacgccggtgcggtcacaggcgcctcagcagatctgagtgacgccgaccgtgccaaagtgctggattatgcctatcagcgctatgtcaggacaggtgcattattcggcacgcccgatactgcaaaagatatggtcgacgaggttatcgccgctgatgtcgatgaaatcgcctgcttgatggattttggtgccgactatgacattgtcaggcacggctttacacatttggcacaattggctcaacattacagttcacctctgttgacaccgtagtaccgacggccgagcacacatttttctttcaagggccgtttcaagatcaccatcacaattttagcaggaaatccaatatggctagcgaactcaaggatctgcgacagcggttggttgaccggctttcggctacggtagagcagaagatttcgtcaatcggatacgtgcccgaagatttggtccgcattgcgggctccggcgtgccagcagaacccagtcatgatgaagtctataaagccccggaggacttgaaagaggccatcaacgaacactacgatttctcgttttatgctcgcgagacgatctgggccgatatgcttgctggcacgcattttcgaaatattggctattgggatgcaaatactgaatctctggatcaggccggccgcaatttgcaggatcaactcctggcactattgcctcaaaaaaccggacggatccttgacgtagcctgcgggatgggcgcctctacaaaacggcttctggacacttaccggcccgaagatgtgtgggccatcaacatctctgccaaacaaatcgaaaccacctctcaaaacgctccaggctgcaatgcacaagtcatgagcgcaacggagatgacttttgaagacaatttttttgatgctgtcgaatgcatcgaagccgcttttcatttcgacacgcggcgcaagtttctggaagacaccctgcgcattctgaagccgggaggccgcttggtcatgtccgatgttctgatgacttcaggggctcggctggagcaatatccggtgttccccaacccggaaaaccacattgccaccatcgaagattacaagtctgtcttggaagaaatcggatacgaaaacatcacaatatctgatgagcggaacaatatttggaaatcgcatttcatggccacaaccaaccggattcacgaaggatttctagcacggaagtataatatcgttgaggtcacagacatgatctggacgtattacgagttggatgcaattaccggcccttgcccgatcctgggcgcatctaaacctcgctaaatgtttagtacttcggatgcctatcgctaggtaggataaaggtactttggttcaaacagagactgacaagcatctttatcgcttgagcgttacgattaagctctcaaggctgcgcgcattggttcccatgtttaaccaccttggcggttcttgcagctcaatgtcagcaaaggcagaaagcaggcactgaaatgccaaacgcccttccattcgggccaaaggggctcctaaacaaaaatgtgcgcccccgccaaaggtatgatgcgcattgcctgtgcgtgtaatatcaaaccggtgagggtccttgaagcgagccggatcacgattggtggcacgaagcaatccaatcaccggcgccccttgcggaattttcacaccaccgatctcgcacgattgtgcggcaacgcgcagcaggaaattacctcccgggtcatagcgcaaggtttcatccgctgcattgcgcgccagatccggctgcgctcgcagccgctccatttcttttgggtgttccaacagtaactttagcccgatcccgatgagggtcacggtcgtctcgtgtccggcaatcaacagagcaacgagatttgtcagtgtctcctcttcgtccagcgtgccgttgtccaggccctgtaatgccagccgcatgaggctgccttcagttccagtgctgctgacggacaattgctctctcagataggatttaaatgccgtcagtgcctccagtccgtcggacttctgctggtcggttaacatcagatcgccaatctggatcaacttcttagaccaatcgctcactgtatctgccatgtcccgcggaatatcgaaaaggcggcagagcacattcaaaggcatgggctgtgcgtaggcatcaatcagattaaccgggcgtccgtcactcggtaaggcagcaatcagcctttcagtttcctcacgcaccatcccttcgagttgagctacagcctgggctctgaaagcgggttcgtaaacaccccgcattcgagcgtggtctatcccgtccacattgatcatttgcggctggaataaggaaaaaagacggaaggctaccggatcccgctcccgaaagccgggatcggaatgccagcctcccttccagttgcgcgaatcccggccgatagccttatttcgcatcgcctccgagaattccgcatgaccaagaataaaataacacccgctcgccgggtcgaaatggatgggattttccgcacgcaacacatccaggcggtcatgtggatcggccaggaagtcagggtccgccagcatcgtccaccagtccgtatctgtttcctccggtacgctcatcgccgatctccctttcctcggccgcttatgatagcgccgtcccggtctgccgaagcgcattaaattgcgctcccagataagaagccgtttgatccatgagatgcaaccctatgtaatcgacatgacggttctgccacttttccagatccgtgccggctacataggtgttgaaagctcctaaggccgggccgcagtagacctgccagtccgttttttgaccagtttccccggccagagccaaacgcattgaatgaatgaaataccaacggaagatcaatgccatcttgacctttggattgcgttccgcacgttcaatttcctcaggtgcagctttgtcgtagaacgatcgggtttcggcatacacgtcttcgaaggagcggcgaaaatacttgtcttcaatctctttgcggatcgcaactggcagcgcttcgaggcctggatgggcgcgccaaagatcgtacagcttgttggcacgtgcggggaagagcagtcctttcttcaagacttgcactttggcacccagttcaaacatatcgccggccggagcataagccgtgtcttgaaccccagtgcgctgcaatacttctttaaccgcctcactggtgccagcctcaggcgtacattgattgatcgatccagtggcaatgtaatccgcccccagaagaaaggctgttgccgcagcttgcggcgtccctattccgccggctgagccgacgcggctaggttgggcaaaactgtgctgagcctgctgagcgtcacgcagagcgatcatcgctggcaaaagcgcacttgtaaccccacggtcggtatgcccgccggaatctgcttcaaccgtcaagtcagaagcaaccggaatgcccggagcaagggaggcttcttcttcagtgatgagaccttgggacagcagtcgctggatcaattccggcgtcgcaggcgcaaggaatgcggaggcaacaccaggatgtgacactttggcaaacacccggtttggcacatctagcgccccatcccgcagtttcgccccctttagacggtatttcaccaacgcttcggttacctccatgaaggccgaagcttcgattacacggatgcccagttgcaggagccgatccaccataagcatttcgcgtctggggtggagtggatcggccaggacgttgacgccaaacaccgagccaggcggaaccgtctccttgatcctgcggatttggaccgcagcgtcctctatcggtactcctcccgacccatatattgccaagagccgggcctgtgccatacggatcaccaaatctgccgaggcaatcccctttaccatggcaccggccatataggcgtggctcaccccatagtcatcccgaaaagcggccgagcccaaatgaccggccgcgatcatccaaccgcctcgccaagatggtttttcaaggcgctcaggttttgcgtatcagccccaaaaggggtcatgaccgcaaaactgcgcgcacgcaaatcatcgcccaacccgtaaaggcacgcggtgcgcaagtttccagccgggccgcaatcgatataggtggccttcgggtattgagcattcagcgccagcaaggtttcatgcaggcggatcggtccgcgcacaaccttccaccaatcccgctcgaccggatcaaatggccgtcctgtgccatccgatgcaccaatcacaggtatctgtgccgcgccccagctaaacgcgcgcagtgcagccctgaaggaggtttcgatcgcctcaatcccggagccgtgaaaagcataccggaccggcaagcggtgatgggagatatcgcgggctcgcagatcatcggcaatgtcattaatgccgtttgtgggcccggtgataacgaaacaacgatcaaatacgacaccagccagctctgaagagccacgacgataaatcggatcagcttcaaattgagctaaatcatcgagcaccatcaacatagcgcccggttccgctttcgactgaattgtccaggcctggcgcagcaacgctggcaaaacctcctctggggatatcgccccggaaacagccgcggcgacatattcgcccaaactgacaccgagcagcagattcggtttcggcagtccttcggcaatcagagtttcagccagcgccacctgaaccatgaacagcgccggatgcgtgtcggtcaactgatcgaatgtgtccccaacatgggcgaaatcatcataaagaacgtctgtgaccggatggtcaagataaggctgtagtgcttcctccatccgcaacatactggcgcgaaaaacgggatgcgcatcatacaagcccctgcccatctggaagtactgagccccctgcccagcaaacatccagatcaccggatcgggcgccaaatcggtcggccatggatgggagaaagcgttcacagtggcgagtccgttgaatgacttaaacaatactgtaaggtattggtgagtggtttgaaatacgcgctatcatattaatagacataggttcgagatgaaggcgtttttattccccgggcaagggtcccagcacatcggaatgggcgaaggcctgtttgagcgctattctgaaatgactgaggccgcagatacggtcttgggttattccattgccgatctctgtctgcgggatcccgacaagcagttgacgcaaaccgaatttacccaacctgctttgtttgtggttaacgccatgatggcgcgcgcgcagcaagacgacagcggagcaccagatatcgccgccggccacagtgtgggcgaatacaatgccttgcatcaggctggtgtggtcaacttcgaagacggtttgagattggttcaaaaacgcggtgccttgatgagcacggcgcccaagggcggaatggcggcagtcatcgggctcacaccggatcgcattgcgacggtcttgcaggataacggctttgcgtcgatcgatgtggccaacttgaactccgacaagcaaacgatcatttccggcctcattgaggacatttcagcggtagaaccgtttttttccgatgctggagcgatgtatattccactgaatgtctcgggcgcgtttcattcccgctacatggctcctgtccaggaggaatttgaagcatttctaggcgagttccgttttgaagcgcccggcatccccgtgattgccaatgtggatgcccgaccttatcaagatggctgcactgctcaaatgttggcgcaacaactgacctccccagtgcgatggcaagaaagtatcgggtacatgttgaatttgggtgtgggacatttttttgaaacggggcccggcaatgtgcttagcaagctggtcgcgggtatccgtaaacagcatgtggtgacacccgtggaaacggagcttccgccccaggccggcagccctccggtgctgcaggaggaaacgcaggcacaggaagcaaaaacacctgtccaaatcgtcgaagactggaacacacagcattctgcgggtatcgatgtccaggtaaatggctatgacggcgtaatgaaaactcgcagcgaagccatccttcttttcggccatcgaccagcagtctacatggaaggctattcaggctattttgcactgtccgatgtgaccccgatagaggcccagttgtcctaatcaggtgcggaatagcgaataaatcccgaacgattttcgctcacacctcgctcggattcttgagtttcaactggctctagagttcccaagggaatttctgttctgtggcataacgttgcaaattggcgcgaatgctcgaatcgccaaacagggaccggttttcagcgatcgccttgtccctactttgaccaagtgacttgtcgaggtccgcgcgataggctttgaaacgtcgtattgcttgcgggtccagccgttcaatacggcgtatgtgttgtgccagaccaagttcgatttccggtaagatggaatccaccaaatgaagggacaacgcctgctctgcattgatggattgggtgcttaaagtcaaataggacgctgcgtgagctccaatccgccgcgtcagaaatggcaggacgcaagctggatgcagcccaaacagcagctcgggcaaagtgaaccgggcatcgggccctgcgaggaccatgtcacttgcggccacaaagccgataccccctgccgttgcctggccttcaacgacgctgagagaaacaaacggtccgagtgccagccgctcccaaagatgataaagcctttcggggtccaccggatctccgccgccgaaatccgccccggtgcaaaacaccgtttgagagccgcgcaggattatcgcggtgcatccggcttcctcggcccggtccagcgctgcatgagcatcctccaccaatgcctctgtgatggtgttaccgctctcaggccgatcaaaccataatgttgaactgcggccattttgggtgatggacagtggcgacaacatccctattccctagtcagaactcaaaaccgtggcgagattaaatcctccaaaccctgaggacaggcacatggcagagttaaaccgtccggactcggggttatctagcacatagttcaaatccgggagcgtcggctggaccagtccatgaatcggcgcgatttgacctgcctccatctgcaggaaagccagggcaatttccacggcaccagctgccgccactccatgcccgagtgcggattttggagccgtgacatgaacagaattgagtaactgggccaccaaagcctgggcttctgcagcatcgcctctcggcgtcccggtggcatgggctgaaatgaaatcgagagaactagggggaataccggcatcagtcaaagctgcggtgatggcctcttgcagcgcattttgtgacggttcaggcccgcgcgtctgggcctggacgcggcccaggcccgatatacgcccataggactgcgggcccagatcactccttgccaaaaccaaggcagcggcactttcaccaaacaagaaaccggtaccggctgcatcgaaagggcggcagcgcggctctggcataagatcaccgctttcatctgaaagatgcggacccatggctcccaaattgcgaagcgcctgcaattccaaccaggacatatcctgcaatggcccgataaccaggcagatatcaagctcaccggagcgaatggcggcagctgccagatgaactgccagcgcaccactggccgaagccccgccaacgctcatgatcgggccatccaataccagttcctcactgatcaaggcggcgacatccgtatccagaaaactgtgccccagccgcggcggcgcaaggttcggcgaggtattaagaagtttgttgcggatcaattccatttcgcgtgactgcaaattgctgccgccaaggatcacacccgtgcggccggagagccggtgttctccggggtctccaaagcccgcatcctgccaggcttctgcggccaccgctgcgcagacctgcccagtcaagccagtggtccgggacgcccgccgcgacaacacctgagggacactgtctggcagctcgatgccaatgaaagggggattcccggcgacttggcgcccttccctttcaagtggtcgaaacaggtttttgccagtaagcaccccctgcagcgcgctggacttgccaaatccatacccgcaagccaaaccaatccccatacaatgcacagtacgatcagtcatgagctgttgttagtttgccgttcaggagatttgccagaaacctggaatgctcaccttcaagcattgaaagatggcctccaggaaccggctggatatccaaaacgcccgcttcagccggccacccccgcatagccgaagaaatctctgcaccttccgcatgaaagacagacgccgcaacggaaactggctctggagtgtagccgtcaacggctttcgcaatgtgtttgtaattattgaaaagcgtgaggagcacctgaaagtcttcccccgtgttttcggccatattttgcaggtatttctccggcgcgcctttgggctcagcactcggcagctccgccgcgagccccatatcccgggcaaatccagcgagaagcgccttttcgtgatcatgcggctggatacgattgtcaatatgggaaagcaccgcagggggataagaatcgatcaatgtcagtgaggccaattcgccgcccgaccgttctatctgccgcgccatttcccaagcgacaataccgccgcttgaccatccggcgagatgaagcggtgcctgcccttgatcaaattcaagatcagccagataggctgttgcggcatccgggatcgagttccaccgatccagccggttcatttccagaccgagaatggaaaatctgggatccagatgtttcatcaaggtccggtaacaaagcaacgtcccaactccgccatgcaccagtacaagacccggaccagacccagcttgtaaattcaccacagatgaacgattacctttggcacgttcaatcagcacagcttgatcggccactgtcggtgcctggaatagctgcgctacggacaattcagccccaagccgcgaacgaatttctcctgcaaaccggattaacagcagtgaatgcgcgcccaattcgaatatgttcgcagtcaccggtactgagggacagtccaacagttcagcccataagctggccaatactttttcgatcctgctgagaggaccctccggtattgatactgatggagcgggcgccccgttcaacgattgccgatccagtttcccggcaatcgtttgcggcagagccgtgacaactcgaatttcacttggccacatgtaatctggcaaactgcttttaagcgccctggatatggcagccggctccagatccgggtccgatactgtgacataggcctgcaacgtggtatcgggcttgcgatccgacaccgtgaccgcagcccgcagcaccccgtcaatccgctccaaaccggcttcgacctcggctaactcgaccctaaaaccgcgaacattgacctgattgtcacgccggccaaggaactcaagctgtccatctgtccgccagcgcgccaggtcaccggttttatacagccggtctgctttacctcccggccccgaggaaaaaccgccttgtttgttctgcgctgcgatgtatccatccgcaagaccgacgccgccgattgcaagctcaccgatcaatccggctggtaaaggctgatccgcaacatctagcacaaacacgttttctcctggcaacggccggccgatcggcagacgtctttcgggtccgtccatttgtgcgcggtaaacaaaagcggtacttccgattgtcgtttctgtcggaccataaacattaacaagagcccgatccgccaaaggactgtcgcaccaggtgctaagggtgttttcggtcaaggcctcgcccccggtcacaaccgtgcgcagactttgcagcagctgccagtcatcactccgcccaagatcgcgcaggacttcatccagaaaagcgggcggcaaatccgcaaccgtaaccgcccagcgctgcacggcctctgcaaagtcaagcgcggaccataatccttcggggcgcatcacgaccgtggcgccacgaaccaacgttgtcagccattgttcaaaagccgcatcgaaactggtttctacgaattgcagaacccggtcctggtcattgaccgcaaaaaggtttgccatcgcttgaatatgatgagccagggcgtggtggggcacttgtacgcctttgggacgccctgatgatcctgacgtgaataggatataagcggcagcggccggatcctgaatgaccggcgttggcagcacgccggccgtggccttgctgatttccgttctctcatccacgcgcatctgacgaatgcttaacaggcttgccgtctttgcatcggtcaacgcaaggacaggagctccatcagcgatcatgtcgtccaaccgtgacgacgactggaccggcgaaagcggcatgtgcaccgccccgacccaccatgtggctagaaccgcgaccagcgaatttgcagaacgtgccaaacagcttgcgaccacatcacccggctgaacacccgcatcgacaagccgggcggcaaggtcaccagcattctgttccaatgcagcgtttgtcaaaacggtatcgccgcaaatcaccgcaggggcatcgggagccatccgcacctgagcgcgccaggctggaataagcgcttcgtcgggtgcaggcggaccgccatgcccccagtcagtaagcacctcatcatccgcacccgccagggacacatccaccagagcccctccgggatccgcaaggaaagttgaaaggactttttgataagcatccgccaaagcagaaacagtatcggatttgaattgcctcgcattataggcaaaccggcaacgcattccttccggtccgggataaacctccagagccagatcctgaacgccctgttggtcaatcccgtcaacgactgagacctccagcgatccgcggtctgtgttttggggtccgaccagcgattgaaaagcaaactgaacccgcggcatcagcaaacggccggtgccggatacttcgcccatctccgacaaaggcaaatccccgtgctccagcgcattcagcattgtctggcgtgtttccctcaccaagtcgcggatactgacctgatctgacagtcggatgcgaagaggcaagagattggcgaagtagccgacagtatgatcgaaactgcgatcgggccggcccaacaccggcaagccgataagcagatcatgggaccctgtcaaacgatgcaagatcagcacgaacgcagccatcatgaattgcgctggcgttgccccgtgtgcggtcgaagcttctgtaatacgccgggctgtatccttgtcgatccaaagcacatgactgccggcctttgaggcgcactccaggtcagcatcccagtcccccggcagacacagctcgttatggccctcgagctcatcacgccagaaagcacggatgttggttccccgttcgctggtcaagaggcgttcctgccagcgctggaacgcatcaaatgaagacccaattgggcgtggaaggcgcacgccctgcagccgggcttcatagagcctcattaaatcatcaatcaggatcattgcggattgcccgtcaaagacgatgtgatgcacgcaaatgatcaagacatgccggtccgccgcctcctggatcaacaggcttctgaccaatggaccattggtaagatcaaatggcaggcctgcgaaggcatgcaattcgttttcgatcacgctcgcaggcgcgccggacaggtccaactcttcaatgggatacgagattccgtcctggacaattcgttgaggcattccgccgttggccgtgaaaacggaggtgaggactggatgccgtttaagcagatccgcgaatgccgcgcgaagcatgtccttgtccaggctaccggccagccgcaatgccatcggcacagtgtagccagcgtcgccgggtgtcttctgatcatggagccacaatgcaatttgccctttggtcagcggcaaagcggtgttcacggcgtcggaggttcctggctgcgcatgtttaggattatccattggcgcatagccgtctttcccggcaatccgggaaagcagcgtggccagtgatttgctttccatgatgtcgcccagaccgacggttaggccacatcgggcctccaaggcttggcacagcggcatcaacatcacggagttcagcccgtgatccaacgcagaccggcgaaagtcaatttcctccggggctatgcgcaggtcgttgatcagataatcccggatacaggtttcggggtcggtagtgtcagaacgggtgtctggctctagctgtgatacagcctcgcgcgcatcaatgccgccgagccaatagcgcgtccggcggaacggataggccggcagtgctaaacgctgcgcgccctcggcctgcagtggggaccagttaagtcgtgcaccgtggacccaggccaccgccagcttcctcaagtttcggtggcgcaacaggagtgacactatttctgcgcctgtcttgcctgtcagcaggccggaaagggcatcttgaccagtcattgtattgccggtgaccagcccatccgcttccagtccttcagccagggcctccagttggtgcagcaatgcgggcacatcacgtgaaatcattgccgcacgctgatccagctggctgcggccggtttgcagggtcaacgccaaatccgccattcgggtctcaggacgggtttccagatatgtcttcaaccgtcccgccaagaccctcaagtccgcttcatcccgggccgaaagcggtatcagatactgatcctgcgcaactgcggccggcggcgcacttgttttgggaggttcttccagaaccatgcaggcatttgtgcccccggcacccacggaattcaaaatcgcccgcaatggctggttcgagcccccactggccgcatctgaccctatcgggcgagcccaggcttgcaactccgattgcagccgaaacggcccagaggaaaaatctagcttagggttcaacgcgtcagttccgagtgttggaaccagggtttccgcttgcatctgcagcacaactttggccagttgcgacaagccggaagcggattctgcgtgaccgatattcgatttgaccgagccaatcgcacagaatttctgttccggcgtcaaatcctgaaaggcttgccgaaaggcggccagttcgatgctatcgcccatcgccgcgccatttgctgcagcttccgcataggtgatagtgtttaccggcacgccagcctggcggatcgtgtcgccaatcaatttggcctgagcggcaacactgggcacacggtagccgttggaccggccgctgtgattgatcccggtcgacttgatcagcgccaggacacgatcgcctgccgccactgcatcgtccaacggccgcagcagcaccgcccccaccccttcagccggcaagtacccgtcgccatcgcggaaactggtgctgtctcggcgcgaccctatgaactgactggctgacagcccgatgtatttctttgggtggatcgaaacgttgacgcccccagcaattgccgcccggcatgcaccggcccttaggctttcgcaagccatatggatggcgacgatccccgaagagcacatcgtatccaccgccaagcttgggccattgaggtccagcacgttggagacacgatttgcgatcgaactcggtgacgacaagactgtcaacgcttcgcgcaatggatctgaacgaacagcgtgatattgctgggtcatagaacccgcaaatacaccgacagcgctctccagatccacgcgcaacgcaggacccatgtaacctgccttttccatcagggcccaggcggtttccagaaacaatcgttcctgcgggtcgagaagttcggcttcatccggcgttatccggaagaaacgtgcgtcaaacccatccacatcggaaagaaaaccaccccatttacatcgggctttgccttcatatgcaccgtctgggtcaaacaaagattcggcgtcccagcgatccttgggcacttcagtgatactgttgcgcccatttacaagattatcccaaaactcctccagatcttcggcaccaggaaaccgtccttccattgcgataatcgcgatatcaccggaaccggcagattgcgtgtcgggtacggccgcttcagcgcggaccggtttagcattgttgtcaagaagcgggtcttcactcggcgcctgatcttctgccgttcccccaggagctggttccagaaggtccacggttggctcaggcacatgtaaagcctccatcagggactgggatgccagatctgttaacgcgccggcagcagcttcaattgtcggattttcaaaaaacagtgtggccggcaaaggcccggtcacggtttcgatcgacgcggtcagggccatgattgccaccgaatccacgccgtagtccaccaacggaacatccgcttccagccgctgcggcgagatacgcaacaccttggcaagttcttctgccagatactcctctacagcatcttgcaagtggaagctgcttggcggcggcgcgggttcaggcccagcgggggcgccagccggctggcctgcgtctgctgctgcgatcaatgccgccaaacggtcaccgtcgccttccagaaccatagtttgcggccatccggccctcacgatcctatccagagcttccaatcccctggctgtggaaaggggaaccaatcctgcgccgtcgcgcatcgcattaaccgccgcagcgtcaagcgtcatgccgccgtccgcccagtaaggccacgctactgaaagagccattccccttggcccgccagggcttagcgcacggcggttccgttcctcgacgtattgatcaaggtaagcgttggccgccgcataatcggcctggcctggatttcccatcgttccggcgatggacgaaaagacaagaaaaagatccagatccaatccgtccgtagcgcggtcaagattggcaacacctgttacctttggcgcaaagactcggcgcagatcttcttcggttttgcgcaagatcaacgcatccgacagcacgccgccacaatgaataaccccatgaagcgatccctgatccgtcgtctggcggatcatagaccggaccgccgctgcatcaccaagatctgttgcaagatagtccgcatgggcccccttgctccgcagttcttgcagcaaagcgttttgtttgggaccagatggggaccggccggttaaaaccagtgaaacccgggacagtgtctgtgccaaatggcgcgccacaatggcgcccagtccgccgcaaccaccgacaatcaggtaacgtccaccttccctccatcccccgccgggctgtgcatcggctacgtcctgttcctcttgccaagtcagtgcttgccagcgcccgtcttttttgcgcaaacgagacttgccgggccaggcagcaacagctttgagatcggcctccaatggaccggaggccggatcatcggtgtcaaagcacagaacctgacaggtcaaacgtggaagttcacgtgccgcgctgtcgagcatgccggccaatgctgcgctctgagaataggaagccggcaataccacctgataatgaactttctgatccgagctctgcagcgccagttccttcagatcccgcaacagggccagcgcctggtctgtaaacgtgttgggatccgctggatcgctggctggaagggcaagctgagcgtgctcctgcaaatttctcattggcccgatatgagcgacacggcgcaaggccgggtcaatagacggcgctgtcagcggcaagtctttccactgcggacgcaacaacagcaaatctgtatgtaacaccgaggtgtgcccggtcgatttggcttcaagtgctctggtttcggtcgtcttggctgttgttgccgcagtccgcaacgcaatgggtgctggctgagccgtggtatcaggccaataaatctcgcgtgcaaatggataggttggcaggctcagccgccgcgcttcgccgccatagattttccgccaatcgtaaactgtaccttgcatccagccgtccagaagaacttcggccgcaagaccatcgggattctccactgttggattgctgacaacccgtgcacgcccggagcgaaccggaccgtcacgtccggccaagaattggcgtaaataccgtgccaactcctcgacagtgctgacttgcacgccgatgcggtgcggcattggttcacggccaacctgcagggtgtaggccagatcacgcaacgaagtctccgctggtgcattttcggcccaatcagcgagcgcgcaggcataggccttgagccggtcttccgcctttgcagacagagtgatcagaacaggcccataagaatatggttcgacggacggaggggggcagtgttcctcgactaccaaatgagcattcgacccacccgcaccgaaggaagaaaccgcagaaacgcgaggcagtgttttaccttcatgcaccggagcgtcccaggtacgcaggcttgtattcacgcgaaacggagttgctgcaaaatcgatgtttgggttgagggtctcagcatgcaaagacggagcaatttcgccagccttgagctgcaggagcactttggtcagccctgccagtccggatacggcctcgccatggccgatattggatttggctgagccgatccagcacggcccctccagaaccggcccatacccgtcattcaaacccttgatctcgattggatcgccgagtttggtgccggttccgtgggcttcgacatagccgatggcccgcgggtctacgccggcctccctcagagcacgggcaatgacatgatgctgcgcctctggattgggcaccgtatagccgttggcgcgccctccgtggttcagcgcgctccccttgatcacaccataaatatggtcgccatccgcctccgcgtctgcaaggcgtttcagcagcacaacgcctacgccttctgcagggacgtaaccatcgccctcacttccgaaactttggcaccgcccattgctcgaaatgaactggcccttgctcaaaaggctgtatttgttgggatgcagattcaggttcacgccgccggcaaacgccatccggacccgtccaagagccagatccgcgcaggccagatggatcgccgtaagtgaactggagcacatggtgtcaaccgccatactcggaccatgcaggttcaaggcataggacacacgattggcaacacctgcataataactggccgtactcattggctcacccgccagactgccttgcaatccaagaagctggtattcgccgtacatgacacccgcatagacaccaacctgtcccggcaggccatcttcgtccaccgactgggcctggagatccccagggcggtaaccggcgtcttccattgcggtccaggcatgctccaggaacaaccgctcttgcggatccatggcttcggccatgccaggtgaaatgttgaaaaacaacggatcaaaggccgccacatcatcaataaacccgccccacttcgaaaagtgagcgtcgatgcggctgcggtcggtcgagaagtaatcttgccatttccaccggtccgccggcacttctgtaatgccgtcgcggccgttgcgcagattgtcccaaaagccagcgatgtcgtaggcctgcggataacgcccggcaagaccaatcacggcaatatccaacccgcccgttttgggctctgtccgcggtttggccgcggcatcaacgctggcaggcgtcccggccgctccccgacccttccgcacaactgtggtcagcgacgggccgtgcgcctcgataaagtggtccaggacagccccaagggtctgatgttcaaaaaagagggtcttggaaagcgttccgaactctttttccagaaccgccgtcagttccatgaccatatgcgagtcgaaaccgtagtactccagtggttcatccagatcgatttcgtccggtggacaggccaacgcttcagaaagaagccgcttgaaataggcggcagcagcgtccttcaggccgtcctgtgccggaacgttcactggatcttgagcgcccaaggcttgatgggcgggggtctttccggcctcgggcagcgctacccgccgggtggaaaagccgttaatccgggtcaagacctgccctgactcatcacagagggcaatgtcgattttttcaatcccgtgcgcggccgaggcgacacttcgccgctcaagatgaacccgcatgcggcttttgttggcggtcagacactgcaggctttcgatcgcaaagggcagggccaagtcaccgctctgctcttccccggccaatccgaatccgattgccgcctgcagagcgccatccataaggctgggatgcaagacgaacggttccactgcagatccgcaaatctccggcaaggacaagtccgccacgacacgcgatccgtcggagaccagccaattcaggcattgatgtcctggtccgtagtgcaaaccggccgtctcaaacagcgaataaatctcgttggacggaatacgccggccagaggggatagcgtcgttattgatgatttccggcggcacttccggcaggtgcgcgattgccccgcggcaatgcaaccgctcacctgaatcaccgtgagacagaatccggaaaggatactcctgccccggaccaggagaacccaaaaccacctgcagcgtctgcggttcggaaatgaccgccggctgcacccagaccacgtctttcaaggcaaggtcgcgtgattgcaaatgcaaacacccggcgctgcgcgccaattccagataagccacacccggaagcaccggctgcccctgtactatatgatcgcgcagaaagaactcatctccggatagcgaaacctcaaacaccccatcagacttgcgagtcagcgccatgccgcttggtaggcttgtgtccttgatctgcggaaccgcagctgagggtttcccgttcaacgtatcaaaccagatgcgttcctttttgaaggacgtccccggcaaaggcacgcgccgtagatcgcgtccgtccctttcagcctcccaatcatacgctgcgccgccaacccaaagccgcgccagctcttccagaggcacgtccttcggagattgggtgaccttgttgtgtcttctggttttataaggaacccgtccgtgccaaaacccgtcttgaccggtgaggttgtcccgggtcgccgctaagatgcgcaaccggtctaccaattccttcaaggattgcgccgcgaatgcgacacgttccgtcattgcatcacggcccgcccgcaaggtgaatgcgatgtcccgcagcagcggggcagcttccattccggaatgctccggcagcgactgaaacgcgctgcttatctcccggatggaaccggcgcgatgcacgagatcatggtcaatggtcaggccgagaaccttttccaccgaccggcgcaacaacgggcgatgcaccggttcgacccccagatcatcgagtttggtaagtggttcgacctcatcgatgtcaatttctagaatatcggccagacatgcgcagagtcgggactcaatcgttggttctaaggcccccccggtgtttgcatagggagtcagagcctttgccaaggcgtctgcgctagccgcaagcccctcacgatcccgcgccgacagaacaatcagttcgggaatctccggcatgtcagcacgtatggtctgtgctggctcttccagcaccacatgtgcgtttacaccgccaaatccgaatgaactcacaccggcccggcgcgggatttcctttccgacggcatcgaccggccgacgccactcctgcgcctgtgggaccaggtagaaagggctatcctttaattttagatagggatttacttcttccggcaggctcggagccaaagtccggttgcgcatctgcaacagaactttcaaaacacctgcgacaccggctgccagttccaagtggccgatgtttgttttgaccgacccgatcgcacaccgcgcttcctgaccggcttcaagagcgtcaaaggctgtcttcaatccttcgatttcaatggggtcaccaagttcggtacccgtgccatgagcctccatataactcagactttgaggggcaattcctgccctgcgcacggctgtttccaccagcgccgcttgggcgcgtggattgggcgccgtcaaggaattcgccttgccgccgtggttttcggcgctgcccaagatgatgccgtggacaaaatcaccgtcccgttctgccgcagttagcggcttgagaaacagcattccgacaccttcgccgcggccatacccgtctgcctgagcgctgaaagtcttgcagcggccgtccggactaagcattcccgctttcgaaaagctgatatgcgtttccgggctgaggacaaggtttacgccgccaacgattgcctggctgcaatcacctgcccgcatggcactgatcgcgcggtgcagcgcgaccaaagcgctggaacaagctgtttccaccggttcgctggggccgtgcaaatcgagaagataacttatcctgttcgggccaacagaaccgacagaaccggtagagctgtggctgtcaatcccgataccgttttcggccatccgtgcaccgtacccagacggggcagtgccaataatcaccgcagtgtcgcttccggccaggcttgacggggcataacctgcatcttcaatagcgcgccagacgtactccatgagcaatcgctgcgccgggtccatcaaggccgcttcccgacgtgaaatgccaaagtgccgggcatcgaattcagcgatcccatcaatgaagccggctcggtttacatcggtcaatccagcggccttcaaggcgcgccaatcccaacggtcttcggggatctcacgtaagcacgcgcggccactgcgcaagttttcccaaaacgtttccagatccggggcgtctgggaaacggcctgccattccaataatcgctatcgcctcggcatccggtggagaagtcggatcgggctgatcaggcaaaggtttttcggtgattttcgcggtatgcctaacgggattttccggaagcaaacccgacaagcagctctcataggtctgcgccagaaaaccggccatgtcggcgatagtgacgtactcaaagaacacggtcggtgtcaggtccataccgtgggcttcattcagccggttggagaaagtggtcattgtaatggagtcaaagccgaggtccgaccactccgactcggcatccagatcctgccgctcgaaccccatgtgttcagcgatgtgctccaacagcaattcttctgccgctagctgcaggccatctgactctgttcgctgggataccggttgcgcgctgacaggcgcggccggtggtgtcagtatgtcgtcaatcgccaactgcgtaccgcacatcaccacttgctgcggcccgccggacagtagtgctgcttcaaattcatcaatgccggcggctgtcgccagaacccccaagccagtgctttcctgcatcctggccaaagcttcgggtgccatacgcatgccgccgtcctgccagggaggccaggcgatgttcagacttaccccgaaccgttcaccttgagcggctttccggctgcgccacagggcaaacgcttccaaaaacccattcgcagcggcatagtccgtttgcccagcgctgccccaaacggcagaagcggacccgaacgtggcaaagaaatccagcggcagatctactgaagcctgatccagcgcccatgttccagcaagtttggcacgccccaccagatcgaaatccgcttcagccttgtccgcaataaagccgtcttttaagacccccgcggcatgcaaaatcccgtcgatgcggccatgacgcgcaacaaccgaacgaaccatggcttgcaccgcatctgggtcgcccaagtcacaagaggtgctgtccacttttagacccaagtcttgtaatcggacgacgagatccgcatccgccgtgctgcgcgctgcaaggatcacagtcgctgcggaagtttcttgtgcgatgcgctctgcaaaacgctgccccaatccaccggtcccgccagtgatcagatatatcccatcattacgccagggagagccctcgccttcaaccttcagtttctcccatcctcgagccaaaatgcccttcgatgacagccggagatgtgatgctccagtaactcgcgccgcctgagataaaagagcaggaagttccagagcagccagatctcccgggcattcgacaagctgggcctgcaaacgggtggattccttgttcgctgttgccaccagccccgccagaccggaaaacaaacccgccgttccatatgcctcatcagattgcggcaccacaatctgcaaaaatcccgtcccttcgccgagcgtcaccgccgccttgaaatcagaaaagattgttttggccgcccgtagatagtcagccacggcattaccgctaacgtgcaccacccgcgcagtttcgccagcacccgaatgtccgtgtgccatgccgcatacaagctgccgtacagcacttggcgtagccgcgccgggcgttaccggatgccagaccggccgggcccgcagcacggtgtcgtttgacgtttcctgcccaagtgccggatccagttcgcggttcgcaaatccctgaaggcgcatcaccaaccggccatcaggaccggtgacgtcaagatcaatgcggggtaggcgcgtgttctgtggtccaacccgtacacaaacacgcagctgatccggtactgtcccgaaaagttccagagtgcccaactcaaatggcaaagaggctgaagaatccgtgtctttttccgccaatcccaaacaggattgaagaacgcaatcgagcatcgccgggtccagcagaaatccctgatcatccgcttcatcgggccgattgatttcggcgtaggcctcgccgtcgggaccacgccagatctgctgcaagccacgatggctcggtccataggaaagaccaagttcggaaaagcggttatagcactgtgccttatccaaaactggcgcggtagtatttgcaggttcggttgccggaaccgtctggccagacccattgcccgtctctccgggtcgcactacaccctggcagtgcagctgcgaaccgggcatgctggtgatacgaaactcaaccgatccatctggcctaccggtacaatgcaccgtcagatccgtggaaccttcggtcacagtacacggctgaacccagacgatcttgtcaaatcgccaggcttcacggtgagaaacgtccaaaaactgcgccgctgctgcgcgcgcgatctccagataggccgcgccgggaagcatggggacgcccacaacaacatggtccttcaaaaaccgttcggcgccggtcagcgttagatcataccggccttcacccggctcgttcttatgtgcggccagcccgaaacccgattttttacgaaacaccgcagatgagcggcggcgcaacggcatttctccggcaggcgcaggaatccagcaccggcgtttttcaaacggataggcgggcaggcgcacttttgccggacggttttcgtgaagcgcagaccagtccagaagagcacccgagacccaggcctcagccagatcaggcaagggctggctcaaatcggccggtgtcgtttcttcccgggatctgcgcctcgtcttgacacatcccttagcaaatccggcctgatcaccgtcccgcaatcggcgtaacgacgcgaccagtgatccaaccgtgtctgccacaaacgccagtctaaacgccatcgggtcacgcccggtttgcaacgtgtaggcaatctgttccaatgagggcagttcatcgcctgcaaatccttccagatgcgccagcaaatccaagataacttgatcaagctgggcttcggttcgggctgaaagcgggatcagcataggccgatccggcctacctactgcagccgtccttgtttcgggaagatattcctcaaccacgacatgggcattcgacccgcctgcgccaaaagaactgacgcctgcacggcgcggaaaggtctgcccatcaagcactggacgcggccaatcactgccctttcgggatatgaagaaaggcgtctgctccagcgaaatcaggggattttggtcttctgaatgcagggttgggaaataacgcccagaacgcaatccaattaccgccttgatcagcccggctatcccggccgctgtttccgcgtggccgatattcgacttgattgatcccaggccacaatgcggcgcgccctcgggagtcttcccgagggcgtcataaagcgacgtgaatgcttgtttcagcccgttgatttctatcgggtctcccaactcagtgccggtgccatggcactcgatatatccaaccctgcgcggatctccgcctgcgtggccatgcgcctccgcgatcaaccgggcctgggcaagtggattgggagctgtcagagacgtcgactgcccgccgtgattttcagaagaaccgcggatcactgcgaggattgtatcgccatcacgttcagcggcagacaatggcttgagcaggactgcgccaaccccatcacctcggacatacccatttgcccgggccgagaacgtcttgcagcggccatcttcgcagagcatgccgaccttggaatacataatgtgcatatccggtgtcagcatcagattggcgccaccggcaatcgccatctcgcaaccttcatgctgcagggccagcaccgcgcgatgcaccgctatgagtgagctggaacaggcagtatcgatcacctggctcggcccggtaatgtccagcatgaatgacaaacgattgggacagaacatatgccccaagctggtcaaatgaagtgcctcgattgatcccgcccgatcaatcatgtgggcgtaatcctggagatttacgccgataaaaaccccgaccggacggccagcgatcgaacttggagcatagcctgcttcgcccagcaaccggtatgcactttggataaaaagccggtgctggggatccatcagctccgcctcacgcggcgacaagccaaaataaagcggatcgaattgatctactgccggggcgacgccgccatatttgaccttggtaaactcgccttttccgggatcatcatagatttgccgccagtcccagcgctcggcaggaatctctgtaatgcaatcgtctccctgctccaggtgcgactgcaactcgcccaaatctgcgctttgagcgaaccggccatccatggccagaaccgcaatcggttcaaaagcagacccgctcacgtgtggggtttcaactgcccccatgtcttcctgatctgtccggaattgtccaggctgcgccaaagccgccttcgcgcttgcaatccaggacgctgcctttttggatcgggacgctggcactgtacggctcgtctcctttggagcacgtttttcagcacggcgatcaggcaacgccagcggattttgcggagctttctgagattgaggaacgcgatcagcctcacggcggtagcgtccatccaagatttgagccaactccttggcgttcttggcttcgaaaaagaccgtaggggcaattgaaacgccgagcatgtccgaaagccgtttcatgatctcggtcacgatgatcgaatccaccccgaaccgggataacggcgataacgtgtcaaaacggtcggaaggtatcttgagacaggcggcgacaacatcgcccacagtatcttcaaattcccggccatctggcaccgcagaccgggatgtctcctgcccccctgccccataagctcggcttcgatccggcgttcagcagcgccggcgcgaccgttgtcgggttcagtcgtcatcgagcatctcccggtaatagcgcgcatggcggataaatttccaaaaatcaacttcccaagtatgcctgcggctgtcaacgaagtagtcctgacccaaatctggatcatcctgggctttcgccgcgacaaacttctgatagccaagggtttgattttcaaaggctctcacgtaactgcggacaaattgcccctgcagacgcattccatcgcccagcccggccgcagcgtttacaaagccaaaaaagaaaagattgttgaggttgcgtggaacgatatggatgaaaagatctggaattccgtctttccagtcgagaatatccggatcgataaaggggaaatgacggtcatagccggtggcatagacgattatgtcgatctcagcttcgtgcccgtctttgaaacgcacggttagatcatcgaaacccgcgacatcgccgaccgtggcaatatcgccatgtccgatatgataaagtatctgcgaattcatgatcggatgggcagcgtcaatcgggtgatccggcgcaggcaaaccgaaatcggtgccatcgaacccggccagcttgaacactttttggatataggccgaggtttcctctttcgaggtgaacttggtgccgagctgcaacatccattgcggtgtcggtttgccgtcgatgaatttcggataatagtggtaaccccggcgtgtgctgtgatgcaccgagacagcatgatgcacggcatccaccgccacgtcgcaccctgaattaccagcaccgatcaccaggacccgtttgcccgcgatctgtgacgggttcttgtaatcggctgtgtgcaacacctcccctgaaaaggttcccggatacggtggtttcgggtagtgcggcacccgctgcgccccgttgcagacagcaacaatgtcataccggcgggttgcccctgtcgacagctccacattccagccgtcgccgtccggttcgatccaagtgacgccagtattgcaatgggcgtggtcataaaccccaaaatgccgcgcataggaccggatatagtccagcatcatcttgtgattggggtaggccggataatgatccggcatcgggaaatccggcacttgtgtattgaacttcggcgaaatcaggtgaagcgagggataagttcttccgcagggcgcatcggtattccagacaccgccaagatcgctttcttgttcataaaggtcatagtcaatcccgccttcggacaattcgcgccccagacctatccccaagggcccgccgccaataacgcaaaccgaaagagccgatgcccgcgttgccgtcatgcctcaacgccctcccattgaatgttctctggaagcgctccaagggacagtgaaaactcccgcaagatcatgagcggtgtcccttgcggatcataaacggtgacgtcaacgttcaagtatcccggatccggatcggatagacgcaccacttcaaagtgcacgtccgaggtcagcggtgctgtgcttgccagcgtcatcagcgaagccggccaagcaacttgcgcggtctccagatccgaaaggcactgcacggaattccagatggcccgcaagacccgcacatcaaaaactgcgggtgcagacaatcctttcatgttcccgacaagccgcccctcatccccgtagagggctgctactccctgcggggcggaaacaggtttgagaccacctcgcaatcgcggcagccggacaggtgccggaaagctggaacacggtgcgccggcctgggctaatagggccagtgcgtcagttgttccggcggcttccaccgccacgagcccctgatcggcagacaagatgcagatttcgttcggatccggtcgaatttcagaactttgcggagctccccagacaatccgggacagggtctgaacgtcacggttcagcacatttgatgcggccccgcgggcggcctccagcatatccaaaccaggagagaggtccgaaactcccgaaacgggcggatgcgcaggcgtgtctggctctggcagtggtttgacatacggcccaattgcgggggcaggccgggcctctggcgcgtcaatccagcagcgatcgcgttcgaacacatagccgggcagattgatccgccgcagactgcacgggaacagattgacccagggtatcggatgcccctgacaaaagagttcggccaattcatgcagagcttcacggctctgcgccttctccagaagtccggaaatctgttgcgacatatccggcagatcaggttcttccgggacgtgtccgcggtaacctggtgttgaatcaaatgcttccaactgccgggcggcatcttgcagatccttgacgaccagcgcgagcctgtgggtgaatgcatgccgaccggtcaacagggtcagggaaatggctgccagctgttgatccgccgcctcgggacttttcagataagctgccaacttgctagccatggcttgcaaggacgattctgtcttcgccgacaaggaaataacatagttccgctcctcagacggtagctgcgcaggcgagtccggagcatcctcgatcagcagatttacattggttccactgatcccgaatgcgctgaccgaaatcaggcgactccggccggcatgcgggcgaggccaatcgcggctctgagtattcacataaagcggagttttctgccatccaagcattggactgggttggttatggttcaggctggcgggcagacggtcatgctgcaaagcatgtacagcccctatggcactgaccagaccagatgccgcgaacgtgtgaccgaagttacccttggtcgtggtcacggcaatgctgtttggttcccgttccgccccggaaaagacatcgcgcagcgcatgggcttcaaccaaatcgcccaattctgtgcccgtgccatgggcgatgacccagtcgatttcgtgaggttttactccggcctgtgcctggacccggcgcaacaaatccacttgtgactgtccgcttggggccgtgatgccatttgtatggccatcatagttggtgccgcttgtgcggatcaccgcctgtatcgggtcaccgtcctcacgcgcccgcgccagagatttcagcaccagtaccgcaaccgcttcgcccggaaccatgccgttggcgcggacatcgaacgtgtagcatttgccatctggcgagagcatgccggcttgtcccatgccgatgtaggcatcctgcgagaccatcaggttcaccccagcggccagtgccacatcgcattcacctgcgcgcaaactctggcaggccatatgggcggccatcaatccggaggaacaggctgtattgagggccagtgcgggaccatccaacccgagaaaatacgatagccgtgctgccagaaccgcattatgcgcgcctgtcaggctaatctgatcggaccgctttatgtaatcactgccatcttcaacgccgacaaaacttccaacccgttggctggccaggtgttctggaccgagggcggcactttcgagcgcaagccagctttcctgcagcaggtgacgctgccgcggatccatccgctcagcctccagcggagatatttcgaaaaacagcggatcgaactcactcagaccgggaacttgtccgcaccatctgctgttggtcttacctggtaccggcggtgttttggcttcgtaaattctgcgccaatcgaaccgctccggggtcacttcctcaaccgcctcccggccctggtccagaatattccataagccacctacatcacgcgcgcccggaaagcggccgcttgttccaatgattgcaattgcatcgtcagaaacagctctgggctgggcaaatgttcgcggctgagtgctctccggtgtcgttactcccacaccaatttcggacaggtgcgcagcaagcttgccaagtgtcgcgtgactgaaaaaaactgatggtgccaagtcgatatcaaagcacgtgccaatggaccgggcaaattctgaaagagcgatggaatcaaatccgaaagaggcgaggttcttatgcgagccaatctctcccgatgacattttcagttgatccgccgctagagacttcaacacggtgagaacatcacccgtctcggcggagggtttggacttctgcggtgtgccggcaaggtggtcgagccgttcggcgttccctgaaagaactagggttcgggtccggccggtaaagaccgccgtttccagcgcctgcatggcctgttcgccttccagcggcacttgaccgctgctcgccagatacaggctctccgactccgcatcggcaagccctctggcacgccagagcggccattccaccgccagaactggaagcgtttcgttattgtgctcggctgcaaaggcgctttggaaacgattggccattgcataatcgcctgaccccaggtctcccagcactgccgagcttgaagaaaagagacacaggaaatcggctcctgaatttgtcagtacctcatgaaggttcttcgtgccttgcaatttgggggcaagcacactgtcaaacccggaagccttagcctcaatcagcggagctgcgccgcttcttccggccagatggaacgctccatccagtctgtcccaacgggagaaaatttgatcacgcacagtatgaagtgcggcgatgtcggtcacatcggctggtagataacaaacatctgcaccaagggcgcatagctcatcaatcagcgcccgatcctcaggccctcggccactcagtacaagccgcgcggacacggtgcgagccaggtgccgcgccaaaactgacccgaccgccccggagccgccgacaatccaataaacaccgcgatgccgccacggagtttgaacatccggcggtgctttcaacgcccgcgaggcacaaatttgcctctcttcgccacgataacgaacacaaacaccggctccggcggtcatttcagcgagcgcatggcgcaccatgacagtcagactggtgccgctgccaaagacaatcgacacgttcagatcaggtagtgctgagcggcaagaacgttgcacgccaaccaaggcatccagccaagcgagatcttcaggagtttctgcatgaccacatatcatcaaagactgcgggcgctgccggccttgcgccaacgcttgaaggaggtggatgattgggccggccacgcggtcttcatcccccagcaacaagagcacatgtgaggctggttctagccaggacagcaaccgtgcagctgcctcgctgttttgaaggtcttccggctcgggcgtcagccataagagatcttccccagcgttcaggtcggcatcggccgccgacatgctttttggcgccagaaccagtacccgtccaaccggccctgaacccggttccagtaagggtgacggctcccattcttccgcaaactgacgaactgaagggagtgcggcagcagcatctggcacatggagcgcttctgggctctctgacccaatccaatgcgttatccgctcgaacggatagcccggcagttcgatcctccgcccctcacgtttcggcgccacctgggcccaatccagatcagcgcctgcgacccaagccttcagaactcgagacaactgccctttggccagccagacttcaatgagatctggcaagtcttcagacagtgcgatacctgtgatttcctctgtttcacccagggtcacattttccggaacctgcccttgcgcaacagtttcaagcaactggatcgtctcggtcagactcgatgtttcaaaggccagacgtgctggcaaccgcgcccggccaacccgcagcgtgtgcgctacatcgctgagacacaaggtgtcctggtttgcccgcagatgctgcgcaagatcacctgccatctgcgcccgaatttcgggtgtgcgcgcagacaatattatgatttcggcttccgctggcgaactgcctggcaatccaggttccgtatcagtcgccggctcttccaatacaagatgcgcattcgacccgccgaccccgaagctgcttaaaccggcacgtcgcggtgccgggcctgacggccaatcgaggctgccgcgcaccagagacaagggagtttcatccagatccagatagggattggggtcacgtagatgcggatttcctgcgatccgattgtgtcggagcatcaagagcagttttatcaatgagacaacgcctgcagcagcctccgtgtgtccgacattcgccttgacggaccccagccagattggcccgtcccgggcgtcgagccccaactccgaaagggcagcttttaggccgttgacttcaaccgggtcgcccaactcggttccggtgccgtgggcttcgaaatagccaatcgaagccggatcgatcccggccctgcgaacgacatcaacaatcagttctttttgagcagttgcattgggtgcggtcggtgaggatgcacgcccgccatgattctccccactggcgcgaatgacgccaagcacgcgatcaccatcacgctgagcatctgcaagaggtttcaataagaccgcgccaacaccttcggaacgcacataaccgttcgcacgggcatcaaaactcatgcaccggccgtcctcgcttaacattccggctcggctggaggctaaagtgatgcgtggtgttgcgagtatgtttaccccgccagccagcgccatgtcgcacataccggccctcaagctttcagtcgcgcggtgaatggcgatcagcgaagaagagcaagccgtatcgattgtctcgctcggaccgtgaagattgaagaaatatgaggcgcgattggcaacgagaaaagaaaatggctctgctgccgaacgcaaatgcccggcctcccgggcctttgccagaagttccgaatagtcgcaggtcgcaactccggtgaagacccccgttcgactgcccgaaacagaatcgggtgcaacacccgcattttcaagcgtggcccagagagtttcgagcatgagacgtaactgcggatcgagcacttcagcttcagcaggcgagatgccgaagtgtgcgtgatcgaaacacgccatatcggcaaggaaaccaccccatttcagcgcagatttatcttcatcgggaccgctttgaaatgcgcgccagtcccaacggtctgccggcacttctgagataagatcccggcctgcatccagagcgcgccagaacgcgtcaaggctctgaacccctggcagtttcgctgccatgccaatcaccgcaataggctcggccgtgtccatcccccggtttaccgaaggggctttgccaattgaaaccgaaccgtcgaaaccggcgctggaccgaaccggcttctcctggtctacaactgcccgcgctggtgcaggcgatgtaacagaagacggccttttttctggctccagagttacactgtgatccttggccagcttgtctgccaaagccgccagatcgggtatctcaaaaaagaccgtcggcattaaccgcaggccaaacgcggaattcacctcattcgccagttctgtgaagctgatggaatcgaaaccatagtcagatagcggtttgtaccgcgtgaccttttgaaccgggatatgctgaactttggcaaccagatcgcgaagccgggtctccagctctgattgatcagcttgttgttcaacagcggcgggctccaaaacgttgttccctgccggatattcaaatcccaggaaccgttcgcgaatttcctcaggcaggccataggcgacaacgagccgggtttcgccgcttgccagagcacgttccagcgcctcaattcccgtcccatccggcatcggcaccattccggtacctgtccgcatcatacgggcgttttcatccgtcatcgccatgccaccgccttgccagaggggccaggcaactgaaagactttggccatggcgttgtccgttcaagacttggccttgccgcagttcggcaaacacatccagatacgcgttggcgcacgcatagtccgcttgcccaacattccccagtacgccggcgacagaggaacataacacgaaggccttgagcggcagttcggccgtggcttcgtccagcgcccgggttcccgccagttttggagcaagaacgcgcgccgccgattcttgccctttatcgcgcaataatccgtcttcaatcagcccagctgcatggatcaccgcatcaagacggccatgcttcgccaagatgtcccgcgccaacaatgtcgcggtactgcaatctgtgacatcgccttgcaagtagagcgcgccggtttccgtgagaaatgcttccgctccggacggcggtgccgaacgccccgtgaggacaacccgttgtccggcagatgcataatgccttgccaggatacgcccaatcccgccaagaccgccggtgatccagatcacgtcaccagcagcgaagtatgccgttcgggaaggaagtggaatttcgcggacccaaccgttttgtggtccgctctctgtcaatcgggacaacatgggcagctgtcctgagttcaatacctgctttaggcctgacgtcagagcgcgatcagatagacttccaggaaccagcaccgcctgcgcacagctggcgggatgttcaagacgaaggcaccgcatgaacccagacagcgacgaagccagactttgatcggggacaatgagcaggaccggccgggcaccccgtacaggatcattcgattggacaaacttcagaatctctgcgaacgcgttctcgaccgtgtcggacaggacacggagatccgcgcccggaaatgctgcccgcaacgtggattgccgatgcgcgtcggtttgcgtcacgaacagcaccggatccaccggcgcagtaccgttcatcagcggtggactgatttcttgccagcacggacctgcaaacagcagctgattggggcccggcaattgctgcttttcagaccagacaagttctagaccgcgaagcgccagaaagaccgaaccgttgtcgtcacacagatccaaatcgagagttacgcgatccgcccccggtgggcctttccgtgccgggcgcagatccacaagcaccttgtccggcagggtaggggtgaattgcgtcaaagagccgatcccataaggcatcggcaaagtggactcttctcgctgggtctgacaccagacgacagctgccaggagagccccattcagcactgccacgcgccggcgcgcccccatttctgcggactgcacccgggcaagcgcgccactcgggccattacgttgctcggccaagctcatcagggacgggccgtgggtagactgcaatacggcatcgcatgcgcgggatgtcagaacgaatggcgtctccgtccggcgggcgtcgagatctacaggcctcgggcgcgtgaaagcagtatctgagcccttctcatggtctgcctggcagtaacgtaccccatcaagagtgatttccaaccgcccaccggtttgttgcaatagcgcagtggcccggccctcgttgatacggagcggttgcggaaagacgatattgcacagtgcgccgtcgccttcgaggtccagcagccggtcaagaaagaacgcgaccggaacaatgcccgagtggtctttcaaaaacgggtcctgcgcatccaatgcgatttcagtaaccgcgggtttggcaacgctgatgggccgggagtacagcccgcgcatttgcaacgctgatgtcccattgggcaacagaatttgcaggtccaccaagccctcgcgccgttgcgccgcgaccagaaccggaccctcccgagcggggcatgaaggtccagcgtttcaagtgaaaatggcaaagcagccggtgccgggttatttggatccgctagcgacaatgccagtgtcgcctgccaggcgccatcgagaagcgcaattggcataacaccactttccgccgtcccgggcaggttcaactctgccagaatttcgtccggcgttgcccagactcgtccaatggattttagtgccggtccatgaacaacacctgcttcattcaatgcaccgtatatggcatccaccgccatctcatgggctgagagccgcgcgcgaattgatggtaaatccaccgctggcggagggccttccaacggtatcaaccgcccttggtgatgcacctggctcgttccgtccggcgcaagactggacaacgcgtaggatccgtcctgatcaaagctttttgcctcaatctccagatccaccggagcctcaacggtcagcggtaccggccataccaaatcctcaaaccgccagcccgtgttccgcgctcctgtcaaccgggccaaggccaaggcaggataagcaacaccgggcacgacaggccggccggcaatccggtgatcacgcaaccaggattcttcgccgttcaagtggagtgtatcatgaccggatttgtccctatcctggtcctgcctatcagatcgccaataccgttccttggcgaacggatacccaggcagatggcagcgctgtccgcgccacccctgatgcagcgcaacgccggaccagtcgatcggagcgcccgcgacccaggcttccgcttgcatggataggacggctgtactagacggcgcttcaggctgcggcccggcacgcgccgtccatttggagggcacttcccctgaccaatctgccgccagtgttcctgccgccaaacctttaaaacggtccaacagttcggtccgggttgtcacaagaaacgccgcacggcattccatcgccatccggccagtccgcagagtatgtgcaatatccgccaggagcaggtccggcacattttcgatcttccgtgccagagccccggcttgcaactgcaggcgttccacatccttggccgaaagcaggatcaactcctgctgcgggtcgccgacgctaacagttggcgaaacccgcaattcgggcgcttcctcgatgacaagatgcgcgttggttccgctgtgcccgaaggaatttaatgccgccaaaaggggctggccatcacgccgggtccaatccgacgtctccgtcaaaggatagaaaggagccccttccaggttgatcagcggattgagcgatttaaagtgcctcaactcaggcattttgcggtgtttcatggccatgagcacggcaatcaacccacaaacccctgctgccgcggcgctatgaccgatatggcttttgacacttccaagggcgcagctgcctggtgtcaaatcatgcggctgaaaggccttgaccagcgcattcgcttcgaccgggtctcccaatttggtgccggttccatgggtttcgacatatgaaatccgccgcggatctatgtcgaaacggctttggacatcggaaatcagtgccgcctgagctgcaccgctgggcgccgttataccgttgctggcaccatcttgattggtaccagaggctcggatgaccccatgaatcgggtcaccgtcgtgcaccgccgcagacaggggtttgagcaccaccatgccggccgcttcggacatcaccatgccgtccgcttcggcatcgaaagtccggcaatggccggtacgggtcagcatctcggtctgggccagcccgatgagaatgttctcgcccatcaccgcgaaggccccaccagccagcgccagatcgcattctccattccgcaagctctcgcaagccaaatgcagagccacaccggaagaagagcaccctgtgttgaccacataggcagggcctttgagatccaggaaataggatatccgcgaggcaacaatcgcgtcggatgccccagtgaatgtgtcgtgcacatacccgctgggctcgcacccgacaaagacccctgtgcggctttcggccagcccgcccggatcgatcccagcatcttctagggcatgccagctttccagcaggattaggcgctggtgcggattcatagacgccgcttcacgcggagataacctgaagaatagcggatcaaatgcatcacggtcttcaagtatcccgccccaacggcagtaggattttccaggttctttgtcttgtgacaccttttcaggacgcatgtaccgccctggcagcggaaccacaggatccaccccgtcgatcatattgcgccagagcgtgtcgacgtcagcagcgccgggaaactgtccggccatcccgatgaccgcaataccatcgtcccagcgctcaagtttccgctgaggatcggtagttacctttggctcaatatctgtttcagacattgcacccccgacggctggatggtgttgttcctcaataaagctgcacaaccgtgccacagtcgtatgatcaaacaaatcagtggtttggagcgtgatgctcagccgcgcaccaatttctcgaacgaacccgacacccaggattgaatcaacgccataatcggagaatggtacatccgaagcgatctcatcacggtcgatgtccaatgcggcggccaaggcgtcttcgatttcggcgcgaattgcttcatttgaaagcagcccgcgacctcgtacttgtgtcccgctgtgtatttcctcctccgaaacagaattgtcatctgtcgtgccgtgctcaagagggccaggatgtacaacctccacctcaaacggctctgacacggcaacccggccatcgctttggccaactacaatttgctggcccaacccatgctgggcctcggctggaaactgcacatgctgcaatccctccaaagcaaacactgtttcccaggtttcgggataaagcccggggctgccgggaatcctgaagtgacggtcttcggccaatgaccagccgtcgatcaacccgaacaggactgaagcaaaaacagttttgtcgctgatatcattcgcaatgaggacgccgccagacttcagcaacgctttcgcgttacggaccgtttcccgtatatcgcgggtggcgtgcagcacatttgttcccagaacaatgtcgtaggccccaatatctaacccttgggccgcgggcgcggcttcgacgttgaaaagttcgaaacgcatgtagggagcgctttgcccgaaccggcggcgcgcatgcgtgaagaacgatttcgacaagtctgtatagcagtattccgcgattgcttcggaccagcgggccagacgcggcaccagagtggccgtcgttccgcctgtaccggctccgatctccagaattcgaagttttgcctcaggatcctgagcacgccgcgcagttatcaccgcgtctacagtatcggcaacgaccgagttgaagaagtcgcaaatccggttgttgctatacagaccttcgatcttttccatctttccagctggaaagagaatgtccgtcacgagagcttgtcctcgcaggatttgcggcaaggctttcagacaatctgttgtcagaatggcaagaacccgcgtatccggagtctcgaggaaggcttgctgcgccttttcccactcggcccagaccgtgtccggtgaaagaagatcatctcctaggagagtaacagctccggccgcatcccgggagatgctgccttgttcctccagaatgttcagcgcttcgtcccaccacggacggaatttggccaaaatggcaaatgtctcgaactcgatcttgcgagacaggcctggacgatcaaagacgtccattttccgcaattgtgccagaagcaggcggcccagccactgatccaatgccgcagcctcgcgtgcaggttccggtggcgcttccctcgtaacgacctggggcaataccggcaaagccgtaccggacaggggcttcattcgaggcgtttccaagacggtctcaatccggtccggccgtgttgttcgactgattgcaatttgcggctgcttcattgcaagggcagtttcaaacagcgccattccagcttcgggatcgattgggacaactccgcgccgggccgccaaagccctcagactgtcagtcacccggacaccgccgccaatgtcccagtagccccaattaacaacagtcactgggcaggagtgtgacctgccaagcgcaaaggccgcagcctccgatgcctggcatccggcaacataggcggccatcccggctggttttccgcatgatgccagtgacgaaaacagcgctacgaaatctggtgtgggaacgcccatcagcgctttgtccagcgcggaaacaacattcaggcgggtcgacaggatatcctgaaacagagtttcggacatttcggcaatcgacttgtcatattctgcgagggtggaaacaattaccccgtcaagcttctcgtaccggttgcgaatatccgcgattgcgtcagccagctctcccgggttgcgggcatcggccgagtgatagctaacggcaccatcataggcagccatattctgtcttatctgcgcagaaagtgccgagcggcccaaccagacaacttgcgctgaaacacgttgcaaaagatgcgtggtccagacccgtcccagagcgccggcgccccctaaaaccaaatagacgccattcttccgataggggatttccggcggcacctctggtagatcgcagggaatcaggcgcggtctcagccattgtccctgacgccgggcaaatccaatctgaccgccttcaagcggcagagtatcaagcaagttgggaaacagtggctctgccgggtgtagatccattgcgcgcaatgtccaaccgggcagttcctgagccagaaccgccaagcagccttgtattgccgcttgctctggatcagcgggctcagcgtcaaaagcaaagccattccgggtgacgagtgtcaagttaccagaggccggaccggtttcgatcagcgccttggcaaagcggaaaaatgttagcggagccgcccccggctctgccaaccaaaggaccgtcccccagttttctcttagtttttttggtgcctcgtccggcggtacaaattgggcatcagggtatgcgttcgccaattgatcccggctcgcgccagtcgcgccgattgccaggacaggtccgatcaacggggcaggtttgtccgtaggtgaaacactttcccaatacgggctgaacgtaacatgttcagggactggatcaggggcgttgggattttctgcgttgtcttcggtgacaacctcatcgaaccacaggcggtgggtatcaaatggataaagtggcaagccaatgcgccgcgcgcccgccattccagccagcgaggaccaatcgatttcttgcccgctaacccaagcctggacaatttcatccagggtgccgttattctgagatcgcagccgcgacgacccgatagcctcttcccttggcccagtctctgaaaggttgacccgcccgcgtgcgcctttatccggaatgcccccgtcttcgacaattctcagctgcgtcagcaaatcctgatgatcctgcaccaagaatgccgcccgttcggccatagcttcgcgccccgtttgaagggttagagcaatgtcgcgcaaatcgggcaattctgttctgctctccagccaaacgcggagattacaggcaacttttttcagctgtgacgatgttcgagccgtaagcgggatcagaactggcccggatttaaacgagcgcggtttggaaggtgtcgcctgatattcttcgaccaccacatgtgcattggctccgccggcccgaaggaagatatgcccgcgcggcgcggcttgtcgtccgcaggtgtccactcggtcaataccgtcggaactcgaaacggggtgttcccgaagtcaatggcggggtttactgcatctgcatgcaatgagggtgcgatttgcccagcgcgcatttgcatcagaaccttggtcaagccggcgagacctgcagcggcctccaggtgaccgacattggatttcaccgaccccaaccagcattggcctggtaacacgttacccgaagcgaaggcttcgaccagaccgtccacttcgattggatcccccaaaggcgttccggtgccatgagcctcaacatagccgattgtgtccgcatctattccggccttgttcagcgccgaccgaaccagtgccgcctgcgcacgcgggttgggcacggtatacccatgggtgtgcccaccatggttgaccgcagtggaacggatcacaccgtgaattcgatcaccatcctgctctgcttcagacaggcgtttgagcaccgcggccccaacgccttcaccggggacatacccatctgcatcagctccgaaactgcggcaccgtccactgcgcgacaacatataggcggaacacaattcagcgtagttggacgaatgcaggtacaaattgactgcaccagcaatcgcgagattcgtactgcgatccaacagcgccgcacaggcctggtggatcgccgtcagccctgaagagcacatggtgtcgatgggcatactgggcccatgcagatccagaacgtaagaaacccgattggctatggagccaaaagaagtgtgcggaaaggccactttgcctgccgcccgctgtgcaggaccgtaaaggtcaaaacctgtcttggtgacaccggcaaaaacacccacattctggtcgtagtgctccttcaggtcctttcgggtcagtgccgcgtcttccagcgcgtgccagacacactgcaaaaatattcgttcttgcgggtcgatatcacgtgcctcacgaggagacatgttgaagaacaggggatcgaaatctgcaaacccttccaggaaaccgccccacttggagtaacttttcccttgagcaacagcccgagtttcatccggttcgaaaaaaccatccagccgccagcgttcttccggaatttcggtgatgcagtcgcgcccctgcgccagattctgccagaatccctccagggaatccgatcccggataccgaccggcgaggccgatgatggcaatggactctgatttctcagcgcgggcatgtgctggcgaagagatgctttctgttgcggaaagctgtgtggtgccggttggacggacagaggtggtattcgaattctgaggcgttatggctgcagcttctttgatccattcgtgacaggctgcgccataggtttttgcaagatgctccgccaggcttcggatggttgagaaccggaacagcagtgtttgagcgcccggcccggccagagattgaagatcgcgcgcgatccgggtgattgtgatcgaatcgatgccgtaatgctgcaatggctcaaccggattgagcgcttcggcatcccgccctagaattggcccaataagggcctttagccggtgctccaggcgctggggcaaatcaccaaccgtgttttgtggcccactttgttttggcccgccggcatccgaactcagccaactcaacgctttgtcttgattgccatagaagacggcagcctccgtcagcccctgctgaagcgcactgtccaacgccttgagggcaattccggcaggtatggggcaaagtcccgtattctggcgcatggccatttctgtgtcggcatccggcggacgcatgccaccatcgtcccaaagaggccagtgcagggccaaactttgaccgaaccgctcgcctgccgcgacagcctgagcccgtttccgggcgaaactgtctagaaagccgtttgccaagcaatatgcggcttgtcccgggctcccgcgcaacgtggcaacggacgacgccatcacaaacaggtccaaatccagacctgctgtggcctggtccaaagcccgagcaccaatcactttgggcgctaacatcgcatcgcattggcgttccaagtccgaggccagcaagccgtccccattcacacctgccaaatgcaatactccatgcagcgcgccgaattttttcagaacctgctggattgcactattcacttccccgggattaccaaggtcgcagcggataaccgtggcatcacaccctgtatgccttaacgaggccaacctttcgggatcaatcgcagagcgggctaataaaatcagccgcgcgccttcagcggcgtgagcaatgtggcgggccaaatgcagaccgattccgcctgcgccgccactgagcacataaactcctccggtacgccaggggctttgcccttccagcgtaaacagggtttgcgcatgccagaccggagtgagaggtgcgccctccgtcagctgccgatgcagcgggccgtcaaaattagcctcactcctcaatgcgccagccaagtcctgaacctgtatggcctcagggacttgtaggacttgaacgcgcaggtccgggatctcctgcgccagggtggcaaaaaaactggtacacccggtccccgagcgtccaatacaatctgcaacagtccagctccaccctccaaagccagatcacgggctttcgccaaaagagcccgcgacaacttcatgtaatgcgacgccggatcggccccggactcgccgggcaaatccgttatccgggcatctggcagcaattccgacaatgtttgctggtgctgtcccagggctcccatgagccaaaccttttggacccctgccggtgctgataccggtattcgattcgcgacctgcctatcttgcgtcagcagcaggctttgcattgagcctgcagatggctgtgcccccggcttgccgggccaaaagatctccttggcaaacggataagtaggcaagctcacccggcgccttggcccagtgtgcaattgcgtccagtcgacttcagtgcctccggtccaggcttccgcgacacgatcaagttgtcgcgttgccagccagtgctccatcagcacactcatttcttgcgacttgagttttggcattgaagccgcgggctggtcttcgagcaattctgctgtagacaagcaagccttcaattcagcgcgcagttcatccagaccggacacgacaaaggctttccggtacaccatatgccggcgcccggtttgcagtgtgtaggcaatatccgcgagcggcgcctccgccttgtcttcgacaaccgccaaaagccgggataaaagctgccgcaagccgtcttgcgttcgcgctgaaaccggaacgatctgcgaagacggctctgctaccggcgaaaccggcatagcagactccggctggaactcctcgacaatcgcatgagcattggtgccgccaatgccaaatgcactgatccccgcacgtctgggagaacctgaggtttccggccaaccctggcgaatggcggccacctccaatccggcatcttcaaaatcaatttccggatttggcgtttcgaaatttatcgagggcggtatctcaccggttttgaccgccatgaccgccttgatcagtcccaccagtccggctgcagtatcaagatggcctatgttgggtttgagcgaaccaatacgaaccggctgcggcgctcccgcggcgcggccataaacagattgaagaccaaggatctcgactgggtcacccagtcgggtgccggtcccatgtgcctcgatatacccaattgaagccggatcaaccttcgcgctttcgagagcacgccggattgcttcagactgaccttgcaccgatggcgcaaagaagcctgccttatcggccccgtcattgctgataccaacgcccttaatcaatgcgtgaatgtggtcgccatcggcctgggcatcgctgagcctttttacaagcacaacgcccagcccttctccagcaacaagtccatcagctttcgcgtcgaaggcgcggcaatggccgtcactggaaacattcaatccgggctggtgcaagtatcctgcccctggcacggcataaaccgacgccgctccgatcaaagctgcgcgggcttccccggccaacaatgcctgccgggcttgatgcagggcaaccaaacccgaagaacagttggaatggactgccatgctcggcccggtaaggcccaactgataggacagcatggttggaacagtcccgccctgccccgcgatccaggcactataaaactcatcatcagacactgcctgacagtcatgcagaagtgtcttatagtgtccgtggctcaccgccgtgaaaacggcggtttttggtaggcttgcggtgctgtgtccggcctcttccatggctttccaggcgtgctgcagcagcaaccgggattggggatccatatgaagcgcggcgcgcgctgaaatgttgaaaaaccctggatcgaaacaggcccgctcggccaacggaaatgccacaggtacgaaatcaggttgagacaactgggcgtccggcacgccagcggcacgcaactcctcaggggtcaaaacctcccgcgcctctcgcccatcaagcaggttttgccaaaaactctgcaaatccaaagcaccaggcaccgcgcaagacaggccgatcaccgccaaaggttcgtcgtccagccgctgagcaagagagggggctgcaaaatggttcagcttcggtgcctcatgcgcagctgtcacctgtttttgcgatgccggcaccacatccgatgtgccggcgcccagatgcctggcttgggcccggattgtcggaaaccgaaacagatcggatacgcgcaactccactccaaagcgctcggaaatccgggccgccaacactgcggcagttaccgagttgccgcccgcctcgaaaaaaccgatgtcccggccaattccggtactgtccaacacgtccgaccagagcgccaggacctctttctcaaggtccatatccggtggaccaggctctatttccggtgaggcagatcgatcgtgacccagatctacttgccgggccgccagcgccatgcgatcgatcttgcccgccggcgttaacggcaggtttgctaaggatatgatgagatccggcaacatataagctggaaggtcttccctcaaacatgcacgcaattctaccgcgggaacagcctctctttctggaacaacataggccacaagttgtgcttccggcccgcttttgcgcaaaacaactgcgctctcgcgcagctccttgtgccgatccagaacacattcgatttctgccagttcgatgcggtggccgcgcaatttgatctgctggtcacgccggccgtgatgaatcagaccgcctgacggactccaggaggcgaggtccccggttttgtaaagacgctctcctgaacggtacggatgtgcgatgaaggattgcgcggtgcggtcggcctgttgccaatatccatccgccaaccccgccccggagatgtacaattctccctgttcaccgacaggcacaagctgcaagtactcatccagaaccagcacttcggtgaaggcaatcggcatgccgatcgtgacggtgtcctggctgccggtcaccgggccgcaagtcgaccagattgtggtttcggtcggaccatacatattccaggcatcaagcttggaattctgaaacaagctattcaaacggtccggcataggctcaccgccgcacaaggccttgaggccgtccggaggctgccaaccagcagcaaagagcatggtccaaaccgaggccgtcgcttgcaagatatcaggctttactcgggaaatctccgacgccagggcatcaggatcctgggcaatttcttccggacaaatatgaaccgacccaccgctggtaattggcagcaaaagctccagcagcgagatatcaaaggcaaacgtggtcacggctagaagccggtctccagtgcccgctccagggcgttgcgccatggcttgcaggaagttcgccaaagcccgatgcggaacctgtaccccttttggacgcccggtgctgcccgaggtgtagatcagataggcgggatcgccgcccttcagcccgacaggctgcggttcaggagcgcacgaaagggcgtcaaccctaaccatggtgcaatcaggttcagccagttgggtcgccatggcatctgtgctgacgtctgccagtatcgctcgcggcgcacaatcttccagaatatgtctcagacgcgcttttggatgggccggatccaatgggacaaagactgcccccgcccgcaatgtccccagaagggccgcagaataattcctcctgcgcccgaggcagagcgcgacacggtcgccgggacgaactccggcctgttgtatcgccgccgcgacccgcaagctttcctgatccagctgctcataagtccaagcgccatcgcaatcgacaacagctgtctcagccgaatgcatatcggtctgcctttgcacgagctgcatcacggtatcagcactgaactccggcttaggcccggtgccccaggccagaagtttggcacgatctgaagtgccaacgatatcgaaactgtcgaggttggcctcaggatcggccaacgcctgttgagccaaatttgttagtgcttcaagccacccctgcacccgttgttcgctataaagatccgggttgtatttcatgcaaagtgacaacgtgtccgaggtttcccgtacctccagtaccagttcgtattcaccctcttgccgcagatcttcgaccagagtcagatcacctgtgacctgcaatctctggtgaagagcaggcagggcatcatgggaaaatgcgttttgatattcaaaagccacccgaaagaccggcggctcacctggaccggagcttaatcccagatcgcgaaccatttgcgcaaacggataggcggcgtgatccagggcatcggcaacctccccctgtaagtgatatgcgagatcacgcaatgttcggccggccaatccctgcatgcgaatcggcagcatattaaccaggtatccgacggtttccgcataacgtggatcatggcgcccgtggtcgggcataccgacgatgatatcgtcatcaccacttaggcgatgcagcagcgtggcgaatagcgcgagacaaagcgatgagagagggcaccgttcagcccttgaataactgcgcatcgcgccagccacggacgctggcaacggtaaggtaagatgcgccccttcgaacaaccgagctgtgtttcgcggcttatccggtgtcagagacaggcacggtaactgtccttcgagccgcctggcccaaaaagcacgggcatcacgcatttcactgccggatgcggccgccttcgccgtagcaacaaaagccgcctgatcggcacctttgttcggcaatatggtggcttcagctcgcaaagatttgcccaattcggcgtcatatgcgtccagaaacgtctgcatgaagagccaaaacgatccaccatcaaagacgatgtgatgaaatgtaatcaaaaggtaggacggtgtgccctgttggccgaagattgttgcccggactggaaggtcacgcgccagatcaaaaggagatttcgccgcatgcctcaaggttgcaagagggtcttcctgcggcaagtcgagctggcgcacatataaagtggccccgtggttttcatcccgcaacggaccacggcggccaactcggaacgtactggtcagaaccggatattgaaccagacatttattcaaggccgactgcacggcggttgtgtcaaacccttcacggctatgcaaacagaccggcaggttatatgcgctggtaccaggctgcgcctgcgcaatggcccaaagccctgcctgaccttgcgacaagggcaaatcgcgggcatgtccccggtcttcgcagacctcgaccgcctgcggctccggatccactgccaatgcggggagttgagttgtgtaatactccgctagggcatcaatactctgatgttccatcaggtcgcggccgcgcacagtgatcccgaacgtccgtgcgacggcccgcaagagctgcatagcgaacaaggaatccacaccgaaatcgtagagatgttgtttggtatctatagaggctgccggcagatctagtacaccggcaatttgatcaatcagaaacggtttgatcccgatttgcgctgaggtgtccggcgcgcgctccagaaccggcttcacccagtgtggccggcaatcaaatgcgtaaccgggcaatcgaatacgccgggcactttgatcctcaaggtccggccaagactgatcgactccagtcacccaagcgcgagccaaggcctcaagcccggtaactgtttcctcagcttgtattccggaccctgccgttccccgaaacaccggccaatccgatgttgcctggccccttgtttccacctccaaggcctcaatcagggctgcggtgtctggcacaacccaggcaattctatatgccaaggcgtcgcgcccctgctgtagggttttcgccacatcacacaaccgttcaggttgttgttgcagatgcttcagaagattagcgatcatccgctcaagacttgaaggcgaagcggctgacagaacgattacctgagggtccgccggagcgtcttgaggacgcgcaaccgatttgcaaggcggttcctgcaacagaatgtgcgcatttacaccgctcatcgcatggcagtgaattccagcgtgacgcggtgtaccgcttcttggccacggtgtgtcatttaccgccagggcgcaagctgcgccatcttcgcggatttcgggatgcacctgatcaaatcctgcaatgccgaagatgcggtccgctgcgaaactgtggaccacctttagcagcgctgcgagccccgaagcggcttccatgtggccaaaggctggtttcaacgtactgacgagacacttcgcatgagggccgctgcccccgcttcttgcccaaagcgcttcattgcccggttgaaggattcccattcggcgatatccgacagcgggttccccatgccttgcgcttcgatcacgcccacctgaccagggccgataccaacgcttcgataacaatcggcaatcaattcggcatgacgtgtcacgctgggggccgccaatgatgccgcgccgcggccattgaaatttacggaagtctggcggatcactgcataaacgctgtccccgtccgccacagcctcagacaacggtttgagcacaacgcacagtgcagcctcggcacgcacatgacctgcggcagtggcgccaaagggagaaaccttgccatccaggctgagttgcccagtttcggcaaggtgccggaacggccctggtgtgagcatcaaattgacacccgcaacaagcgcctgggaaatttcgccctgacgtagggcctgcactgcccgatgcagcgccacgccggcgctggcacattgcgcctcgatcacttcgctggggccatcaaaatcgtagaagtatgacaggcggttggccaagagacatgattgtgcatatccggcatccgggtcatgccctaggctggcacaaagccggtcatattcgttgtcttgagccgcgacgaaaacaccggtacggctaccccgtaaattgcgcgatgcgtagcctgcatcgtacattgccccgagtgcggccatcagcaacagccgctgtcgcggatccatctgatccgcctcacttttcgggatgtcgaaaaacccggcgtcaaagccggccggatccggaacgaaaccgccatagacaaagggcgcatcggccgcaggcgccgacaaccgctctccgagtgatctttctgcccgtttttcaataaggcaggcgccggtttccaacgcagcgtaaaatgctttcagatcctcacagccaggaagcatgcctgatgcgccaacaatggcaatcggagcgggctctcgcgccgctccgtcatgatcttgcggtggagtattactaaacttgtcctggcattgcatggatgcgaggattgccgcctcaatttcgttccatgcttcgtcagaattcatgacacgctttccatacatcaatattcaaaaaccggacacaatcctggccatcaatagcctgagcaaggctttttcgcccttgcctcgcgccgttagccaagcatctgcctgctacccagcagtcgcagaattgtgttgagccgaattacaatcggcgggccgtaatcatggtgtatccgaggttttctgccatatattcaaatagatagtaccagttgtcgacggctttttctgcctgatctcccatgagccggaccacgtcactccacttttcctgcacggcttcacgcaacttggtttccagccatggcatgacgttttcggaaatgtcctcaatgttcagaagttcaaatccggcatctgccatcaaggcgggatagcgatcttctggaacaaagaccgaatggatatgctcatggacaaagtccatgaattcaggtgtcgtgtgaggcagagttggcaggtcggtcaggacaaggcctgcgccgggtttcaaaagccgggcggcttcgcccaaagcttctgcatgacccatgtgaaaaatcgattcaaaaaaccagccgccatcaaaactcttgtctggcaatgggacgctgcgggcatcggcttgtaaaaaatccaatctgtcggagaaccctgcctgtgccgctttctcgcctgcaatacgatgctggtagccactaatcgtcactccggtgacatgacaacttcgagcttgagcaagtttcaaggcgggatggccaataccacaacccagatcgacaaaccgttcaccgggaccaatttcggtccgatcgatcattcgatgacacatggcttccgctgcggcaccgaaacttgcatcccgactgtcttcgtcccaataaccccagtgtaagtgttcatcaaacaggatcggtcccagtcgcagagccggtgagtcataatgatcttcgaccgtatcattgctagcgccggtagtctccaaagtactgcgggacat

SEQ ID NO: 3 - protein, 80 aa, Labrenzia sp. PHM005
MSQTDPFETVKRNVQEVLPELEPDMIQPESILVDLGANSVDRMDVITLSMEDMGIAIPLMSFAKAVTLRDLAEILAASKV

SEQ ID NO: 4 - protein, 425 aa, Labrenzia sp. PHM005
MNTAGIEAVGVYGGSVYLDVSELAQYRGMDLQRFENLLIRQKSAALPYEDAVSLGVNAAKPVIDALSQAERDQIELLITCTESGLDFGKSLSTYIHHYLGLSRNCRLFEIKQACYSGTAGYQMALNFILSQTSPGAKALVVATDLSRVLVDETSDELTMDWEYFEPSGGAGAVALLVSDQPRIFQSDIGANGTYCFEVMDTCRPMPDSEAGDSDLSLLSYLDCCEQSFAAYRARVEGVSYQDSFNYLAFHTPFGGMVKGAHRHMMRRLLRSRPDEIDVDFETRVAPGLRLCQRIGNIMGATVLLSLTGAVLYGDYRTPQRIGCFSYGSGCASEFYSGVSTADGQRRLQDAPIQKALDLRHKLTMPQYEALLEGCKAVPFGTRNHQPDLDQVPDMKSCIADQSAQLGYQRLFLKEIKNFHREYDVL

SEQ ID NO: 5 - protein, 1166 aa, Labrenzia sp. PHM005
MTGCQSKRAGLSPLALLLNAAGRGLFPAAGVTFRPDCRAEDLEASLEPADFNIRPAAVDDIDTLHMLETVCWPKELQTPTKTLASRVAIDPNGQLVLTLDGSPCGVIYSQRINSVEALTSSDMDKVDSLRDPSGSILHFLAINILPSVQDRGLGDALLEFILHYAALAPGIKSAAAVTLCRDFTGRTLSDLNEYLRRKTPLGTVADPVLRFHELHGGRIQHPVPNYRARDTRNLGAGVLVTYDLNKRRRSHAPQPRQKIARTDIANRVNSAIRSALGSSSDQFEKDTPLISMGLDSAAILGLADCLQAECGSTLTAAQLFKHNTAEKIIAFLHNELPSSGLSKPTLLPAQTSCPADGGSDQSVAIIGVSLRMPGGIETPQALWELLDLGGTVITPVPSDRWSWPDGFRPQGAAYGGFLQDPARFDAAFFRISPHEAEAMDPQQRILLELAWHGLEDAGLSATKLAGSSTGVFVGASGSDYQRAMDAAGVPVQPHHSTGAALSVIANRLSYALDFTGPSLVVDTACSSSLVAVHQAVAALQERTCGLALAAGINLILHPATSQAYQSAGMLSPSGLCRSFGSGADGYVRSEGAVLLVLKPLAQALAEGCRVHAVIRGSACNHGGMTSGLTVPSPDKQTELLSAAWHNADIKPADLDYLEAHGTGTKLGDPIEIEGMKTALAEFDDSQPNPPEQHACLTGSVKSNLGHLEAAAGLAGLCKVMLALRHERLPASLNASPQNPEISLNGSNLAIADTARDWPKGNRPRISGVSSFGSGGTNAHIVVAEPPDAPDGVIDTGPQLFVLSANTPERLMALAVHWQEWLKKQPHDLNIPALCHASRHRRAALPARFATKVSSRADLEKALHQAAQKNPASSQAKPKFLEHLKGDAGQAFLQALAKEGDLSALADLWCAGVPVDWSLIDSTPPEQPVPWIDLPLYPFDKTRFWALGKAPAVPQDRAAATAELYAPVWQELAASKTQMPEPDLLSGPFALKAAQLLKLDPSESRNSETNAIGENMHVLWSSAPRPSDSGETLEEFREFQDFVAGLPRQLSRLRLTVVTWNGQAVYGNEPVDAEAAAISAFTHVLAQEKPEWDIRTFDLDSCDPPSWSSLAESNETRSAVRAGKAYGLRLAMADPLPDTGQSHLREDGVYVVIGGAGALARPGVKRF

SEQ ID NO: 6 - protein, 3219 aa, Labrenzia sp. PHM005
MIHAITGPSDQPILDSEPENLTRVMAAKTHGLIQTAHTFAALDLDFFLVFSSIISLEQPGGFGGYAASCAFADAFVRGLDSQTPYPVRCLNWGHWDVGVARNLPEATKIRLDNAGVVPITAQDALKHCDTALNAPLPQLAILKWNDPARHPLVDSQVHMRLSRKAPARSLPAATNELNTRLQEIERHGLFAHPELEAALPGAIAAELDRHGLRTSLPDTAPWYLRRWHKATKRLLAQGNTGENWDATARRLRADADLAPAINLVTACLARLHEVLTGQTPATDVLFPGASLDLLEPVYRGTASADLLNDVLADTLAETLRADLRDQPENTSLRVLEIGAGTGGTTARVLPCLSELAGQIETYDYTDLSRAFLQHAQQAFAPSAPFLKSLRFDVEKSPESQGLQPGSYDAVLATNVLHATPDIRQTLRHTHALLKPGGVLLLNEIVTPSVFAHATFGLLEGWWKSCDPGLRHPDTPLLSAESWEKLLLANGFTAVEMLLNSSTALGQQVFAARSDGCFEYRKAEIDTTRRQPETLEPRILKNTVSELPLEDLENPQAAAARLLTEIVASALQITEDQLDPWTPLGDYGLDSILNAQVTARLRELVPDLDTTFLYQYQTIADLSQALVQKHPEAFEQIGHTTCGEADVASPSTVSASKRTAGNEQQDIAIVGMSFRFPKADTPEEFWTLLSQGQSAVTEIPPDRWQLDGFYESDPDKAVDGWKSYSKWGAFLERVTAFDPLFFGINPKEAAAIDPQERLFLQTAWAALEDAGFPRQRLADELARSVGVFVGITRTGFDLFGPDLWQAGQKVYPHTSFSSAANRLSWFLDADGPSMPVDTMCSSSLTALHQACASLKTGECRLAIAGGVNLFLHPTSYIGLSAMRMLSPDGRCSSFGAGGNGFVPGEGVAALVLRPLAEAQAAGDQVIGVIRGSAVNHGGRTNGFTVPNPRAQSSLVREAMSRAGLEPGQISYLEAHGTGTEMGDPIEITGLTEAFAGREQGLAPCAIGSIKTNIGHLEATAGLAGVIKVLLQMRHRQIVPSLHSSSLNPKIDFEHAPFRVAQDLTPWSPAKGRRIAGVSSFGAGGTNAHVILEEAPDIPEKSATDPAPNEPIALVLSAHDEPRLRAYAARLAKFLTSPNAPPLALAAQSLQLGREPMRHRMAAVVSDKAQAVAVLQAVAENRPLPDKTFLRDTRRYKGQCPSSVESEDLGELTDAWSKGSKIDWAKLHQRRQTVSLPTYPFDEKPYWFADTAPVGGPMDVPSSEDAFRELKPASRPSPVRRTLPRLDTAPAQFEPHRRSQKLRLSSLNPASETPPAEIELDINGIGRVRLEPASPPPNLSTGNAMKVLVVEGLQHWNGDRLGLLHELDQLSQPVILTVSASSLPPIPDTLLTAPAFEQAQEMANATARCPAATLATLKNHIRNQPSWPDIAGIPAEWMAGSGWPVSSPEPAPSGGAIPLQSEVVQLHDMGGGVAQITMAERDAQNTFTPAFVTGVLEAFDKVESSAAFKVVVLTGYEAYFACGGTREGLLAIQNGQARFTDEQSYARPLRCPIPVIAAMQGHGIGAGWAMGLYCDLAIYSEESCYQSPYMLYGFTPGAGATTLFPARLGRQLANEILFTAQSFPGHILAQKGLTAPVLPREEVLPQAHALARSIAQNPRETLMARKSTQTAEFLHMLPRLFEAELALHESTFVGNSDVLEQISEHFADKQMTQKPGASQKEARNTSALKTQLRMMLAEELDIPPDRIDDDTPFVDLGLESIAAVIWVRKIGEELGAQIGATSVYSHPNLAAFTELVAEKGGQLAEAVNKTTAPPSEPPKAAIPADPEERLLPSDSSDLFVWLQASLETELSIPSGTLDPDRPFVELGLDSVTAVTWIRQVNDALGTKETGTVVYHHTNLTELAAYLAGIAGKTPTTRTTSLPYKLEAPVRSALPRLENLAPFQDERPGIAIVGMAGRFPEAPNVSSFWQNVLAGRDCVYEIPATRWSIDAYYDPDRQAPGKTVCRRMGAIEDIDAFDSLFFGISPAEAELMDPQQRLFLETAWEAIEDAGHAPSTLAGTRCGLFVGTENGDYARIAGDAKPEALALTGRSVAMLPARAAYALDLQGPCLAIDTACSASLVAIAQACASLHDRHCDSALAGGVNVLTGPEIHVAMSHAGMLSPSGKCNSFDSRADGFVPGEGVGALLLKRLEDAQANGDDVYAVIRGWGVNQDGRTNGITAPNPAAQTRLQTELYHRFHIDPARIGMVEAHGTGTALGDPIEVEALKRSFAQFTDRKNYCALGSVKSNIGHLATAAGVAGAIKATLALKHRKIPASIHHDQLNPHIDLKDAPFYVPRTAADWTAGPDAPQYAAVSSFGYSGTNAHLVLEAAPARPVPVTQTQAVIVPVSARSLECLTEAVTRLSTYLGTGAGQTVPLADLALTYQTGRDTFDQRVAFLADSHDSLRAGLEQFLNEPEHAGGVVYSNDMPPTLRDTATAWIEGKTIAWPVVAGASRRHGCPTYPFAKERHWVSDAPVELPEAAPIPSKETPLQPEAEDTAVDPDWRERLKQRFARPITLLSDDPKWIGSMASLLSALGAAPGGPGQPDLRIKSNLREAEGSVFCDTHLGTRLPGNEQVDLLILTELPSDPGLIPQHALIVSDDNRDDIESHCQRLIQEWLRLEPDGSKDTLHVQFRNGRRLVAAKPLDPADGACILRKTWQRTPLADQKTAPSDKNVCLIGRGPKFEALASGLEAHFQSVTLRDTPPEGAMAAWDVFIDAAALTEVRDNDPDDPDRRHWIQSLMREGRDLNLLHLTCDVIPFRSVSRNLAGARQAGLVKNLRAEYRFAESRWLDLDMAQVADTAGLAKLIAAECASAGPVSEVCYRGGARFAPVLEAPEPVASPSVHLNAEGLYLISGGTRGVGLTLAQDLAAQGARHLALIGETPLPPMQDWPSLIAAADTPAEIRSQLSILQALSDQLETLEILHACVSDAAKVSAWLSSLRKRGLPLSGVIHAAGRYSEVDPPGFAAKSADHMRAVLTAKADGLETLHSLTKNDPLSFLLVLTSITGLVPHFARGALDYAMANAYADLFAAKAHELDGGRTRSTILSDWTQSGAFCRVRPEKAKSVQKNFDQIGLKTLSDAEGCALIRRALSPTAETGTILGLIAEDRFAAARPGLLLAGTLNDEALDMNTQLARWEKIRSRGDLVTIEDVTSVIGLEQIRELPPRKCFASTGSCLAPLK

SEQ ID NO: 7 - protein, 97 aa, Labrenzia sp. PHM005
MLRLHRIMLGPTEVVPPEAEDESLPDMIAGIVCNVLKLKEIDHNTPLQNYGLDSISGMILSTRLEIALDMTVDPRTLIDHPSIAALSAYIQKAREAA

SEQ ID NO: 8 - protein, 373 aa, Labrenzia sp. PHM005
MSQSIEELLGVDTLPKPSRRQNMRFSCLFFSDVRTDISYAEKYRFLGDVTRFADQTGFEAVYFPERHFHEFGSVFANPAIAAAHLIPQTQNIRFRTAGVTIPLHHPAEIVEWWAMNDVLSGGRVDLGFGSGWAKGDFIYAPENFEDRRKICSDGIETIKRLWRGETLAFPGPGGDVVDITVYPRPIQSDLAVWLLITQNEDAFIHAGKMGYNVFTMLYGTNLENLSQKIALYRKARQEAGHDPVSGRVTLTLHTLLLDTMDSVLAAIEVPFRQYIQSSLNAHVNAGAVTGASADLSDADRAKVLDYAYQRYVRTGALFGTPDTAKDMVDEVIAADVDEIACLMDFGADYDIVRHGFTHLAQLAQHYSSPLLTP

SEQ ID NO: 9 - protein, 318 aa, Labrenzia sp. PHM005
MASELKDLRQRLVDRLSATVEQKISSIGYVPEDLVRIAGSGVPAEPSHDEVYKAPEDLKEAINEHYDFSFYARETIWADMLAGTHFRNIGYWDANTESLDQAGRNLQDQLLALLPQKTGRILDVACGMGASTKRLLDTYRPEDVWAINISAKQIETTSQNAPGCNAQVMSATEMTFEDNFFDAVECIEAAFHFDTRRKFLEDTLRILKPGGRLVMSDVLMTSGARLEQYPVFPNPENHIATIEDYKSVLEEIGYENITISDERNNIWKSHFMATTNRIHEGFLARKYNIVEVTDMIWTYYELDAITGPCPILGASKPR

SEQ ID NO: 10 - protein, 414 aa, Labrenzia sp. PHM005
MSVPEETDTDWWTMLADPDFLADPHDRLDVLRAENPIHFDPASGCYFILGHAEFSEAMRNKAIGRDSRNWKGGWHSDPGFRERDPVAFRLFSLFQPQMINVDGIDHARMRGVYEPAFRAQAVAQLEGMVREETERLIAALPSDGRPVNLIDAYAQPMPLNVLCRLFDIPRDMADTVSDWSKKLIQIGDLMLTDQQKSDGLEALTAFKSYLREQLSVSSTGTEGSLMRLALQGLDNGTLDEEETLTNLVALLIAGHETTVTLIGIGLKLLLEHPKEMERLRAQPDLARNAADETLRYDPGGNFLLRVAAQSCEIGGVKIPQGAPVIGLLRATNRDPARFKDPHRFDITRTGNAHHTFGGGAHFCLGAPLARMEGRLAFQCLLSAFADIELQEPPRWLNMGTNARSLESLIVTLKR

SEQ ID NO: 11 - protein, 455 aa, Labrenzia sp. PHM005
MIAAGHLGSAAFRDDYGVSHAYMAGAMVKGIASADLVIRMAQARLLAIYGSGGVPIEDAAVQIRRIKETVPPGSVFGVNVLADPLHPRREMLMVDRLLQLGIRVIEASAFMEVTEALVKYRLKGAKLRDGALDVPNRVFAKVSHPGVASAFLAPATPELIQRLLSQGLITEEEASLAPGIPVASDLTVEADSGGHTDRGVTSALLPAMIALRDAQQAQHSFAQPSRVGSAGGIGTPQAAATAFLLGADYIATGSINQCTPEAGTSEAVKEVLQRTGVQDTAYAPAGDMFELGAKVQVLKKGLLFPARANKLYDLWRAHPGLEALPVAIRKEIEDKYFRRSFEDVYAETRSFYDKAAPEEIERAERNPKVKMALIFRWYFIHSMRLALAGETGQKTDWQVYCGPALGAFNTYVAGTDLEKWQNRHVDYIGLHLMDQTASYLGAQFNALRQTGTALS

SEQ ID NO: 12 - protein, 337 aa, Labrenzia sp. PHM005
MNAFSHPWPTDLAPDPVIWMFAGQGAQYFQMGRGLYDAHPVFRASMLRMEEALQPYLDHPVTDVLYDDFAHVGDTFDQLTDTHPALFMVQVALAETLIAEGLPKPNLLLGVSLGEYVAAAVSGAISPEEVLPALLRQAWTIQSKAEPGAMLMVLDDLAQFEADPIYRRGSSELAGVVFDRCFVITGPTNGINDIADDLRARDISHHRLPVRYAFHGSGIEAIETSFRAALRAFSWGAAQIPVIGASDGTGRPFDPVERDWWKVVRGPIRLHETLLALNAQYPKATYIDCGPAGNLRTACLYGLGDDLRARSFAVMTPFGADTQNLSALKNHLGEAVG

SEQ ID NO: 13 - protein, 375 aa, Labrenzia sp. PHM005
MKAFLFPGQGSQHIGMGEGLFERYSEMTEAADTVLGYSIADLCLRDPDKQLTQTEFTQPALFVVNAMMARAQQDDSGAPDIAAGHSVGEYNALHQAGVVNFEDGLRLVQKRGALMSTAPKGGMAAVIGLTPDRIATVLQDNGFASIDVANLNSDKQTIISGLIEDISAVEPFFSDAGAMYIPLNVSGAFHSRYMAPVQEEFEAFLGEFRFEAPGIPVIANVDARPYQDGCTAQMLAQQLTSPVRWQESIGYMLNLGVGHFFETGPGNVLSKLVAGIRKQHVVTPVETELPPQAGSPPVLQEETQAQEAKTPVQIVEDWNTQHSAGIDVQVNGYDGVMKTRSEAILLFGHRPAVYMEGYSGYFALSDVTPIEAQLS

SEQ ID NO: 14 - protein, 245 aa, Labrenzia sp. PHM005
MLSPLSITQNGRSSTLWFDRPESGNTITEALVEDAHAALDRAEEAGCTAIILRGSQTVFCTGADFGGGDPVDPERLYHLWERLALGPFVSLSVVEGQATAGGIGFVAASDMVLAGPDARFTLPELLFGLHPACVLPFLTRRIGAHAASYLTLSTQSINAEQALSLHLVDSILPEIELGLAQHIRRIERLDPQAIRRFKAYRADLDKSLGQSRDKAIAENRSLFGDSSIRANLQRYATEQKFPWEL

SEQ ID NO: 15 - protein, 411 aa, Labrenzia sp. PHM005
MTDRTVHCMGIGLACGYGFGKSSALQGVLTGKNLFRPLEREGRQVAGNPPFIGIELPDSVPQVLSRRASRTTGLTGQVCAAVAAEAWQDAGFGDPGEHRLSGRTGVILGGSNLQSREMELIRNKLLNTSPNLAPPRLGHSFLDTDVAALISEELVLDGPIMSVGGASASGALAVHLAAAAIRSGELDICLVIGPLQDMSWLELQALRNLGAMGPHLSDESGDLMPEPRCRPFDAAGTGFLFGESAAALVLARSDLGPQSYGRISGLGRVQAQTRGPEPSQNALQEAITAALTDAGIPPSSLDFISAHATGTPRGDAAEAQALVAQLLNSVHVTAPKSALGHGVAAAGAVEIALAFLQMEAGQIAPIHGLVQPTLPDLNYVLDNPESGRFNSAMCLSSGFGGFNLATVLSSD

SEQ ID NO: 16 - protein, 5897 aa, Labrenzia sp. PHM005
MPDGREFEDTVGDVVAACLKIPSDRFDTLSPLSRFGVDSIIVTEIMKRLSDMLGVSIAPTVFFEAKNAKELAQILDGRYRREADRVPQSQKAPQNPLALPDRRAEKRAPKETSRTVPASRSKKAASWIASAKAALAQPGQFRTDQEDMGAVETPHVSGSAFEPIAVLAMDGRFAQSADLGELQSHLEQGDDCITEIPAERWDWRQIYDDPGKGEFTKVKYGGVAPAVDQFDPLYFGLSPREAELMDPQHRLFIQSAYRLLGEAGYAPSSIAGRPVGVFIGVNLQDYAHMIDRAGSIEALHLTSLGHMFCPNRLSFMLDITGPSQVIDTACSSSLIAVHRAVLALQHEGCEMAIAGGANLMLTPDMHIMYSKVGMLCEDGRCKTFSARANGYVRGDGVGAVLLKPLSAAERDGDTILAVIRGSSENHGGQSTSLTAPNPLAQARLIAEAHGHAGGDPRRVGYIECHGTGTELGDPIEINGLKQAFTSLYDALGKTPEGAPHCGLGSIKSNIGHAETAAGIAGLIKAVIGLRSGRYFPTLHSEDQNPLISLEQTPFFISRKGSDWPRPVLDGQTFPRRAGVSSFGAGGSNAHVVVEEYLPETRTAAVGRPDRPMLIPLSARTEAQLDQVILDLLAHLEGFAGDELPSLEQIAYTLQTGRDPMAFRLAFVADTVGSLVASLRRLRDGDQAGFAKGCVKTRRRSREETTPADLSQPLPDLAEAWVSGALLDWSALHENRPAKVRLPAYPFEKRRCWIPAPAGEMPLRRRSSAVFRKKSGFGLAAHKNEPGEGRYDLTLTGAERFLKDHVVVGVPMLPGAAYLEIARAAAAQFLDVSHREAWRFDKIVWVQPCTVTEGSTDLTVHCTGRPDGSVEFRITSMPGSQLHCQGVVRPGETGNGSGQTVPATEPANTTAPVLDKAQCYNRFSELGLSYGPSHRGLQQIWRGPDGEAYAEINRPDEADDQGFLLDPAMLDCVLQSCLGLAEKDTDSSASLPFELGTLELFGTVPDQLRVCVRVGPQNTRLPRIDLDVTGPDGRLVMRLQGFANRELDPALGQETSNDTVLRARPVWHPVTPGAATPSAVRQLVCGMAHGHSGAGETARVVHVSGNAVADYLRAAKTIFSDFKAAVTLGEGTGFLQIVVPQSDEAYGTAGLFSGLAGLVATANKESTRLQAQLVECPGDLAALELPALLSQAARVTGASHLRLSSKGILARGWEKLKVEGEGSPWRNDGIYLITGGTGGLGQRFAERIAQETSAATVILAARSTADADLVVRLQDLGLKVDSTSCDLGDPDAVQAMVRSVVARHGRIDGILHAAGVLKDGFIADKAEADFDLVGRAKLAGTWALDQASVDLPLDFFATFGSASAVWGSAGQTDYAAANGFLEAFALWRSRKAAQGERFGVSLNIAWPPWQDGGMRMAPEALARMQESTGLGVLATAAGIDEFEAALLSGGPQQVVMCGTQLAIDDILTPPAAPVSAQPVSQRTESDGLQLAAEELLLEHIAEHMGFERQDLDAESEWSDLGFDSITMTTFSNRLNEAHGMDLTPTVFFEYVTIADMAGFLAQTYESCLSGLLPENPVRHTAKITEKPLPDQPDPTSPPDAEAIAIIGMAGRFPDAPDLETFWENLRSGRACLREIPEDRWDWRALKAAGLTDVNRAGFIDGIAEFDARHFGISRREAALMDPAQRLLMEYVWRAIEDAGYAPSSLAGSDTAVIIGTAPSGYGARMAENGIGIDSHSSTGSVGSVGPNRISYLLDLHGPSEPVETACSSALVALHRAISAMRAGDCSQAIVGGVNLVLSPETHISFSKAGMLSPDGRCKTFSAQADGYGRGEGVGMLFLKPLTAAERDGDFVHGIILGSAENHGGKANSLTAPNPRAQAALVETAVRRAGIAPQSLSYMEAHGTGTELGDPIEIEGLKTAFDALEAGQEARCAIGSVKTNIGHLELAAGVAGVLKVLLQMRNRTLAPSLPEEVNPYLKLKDSPFYLVPQAQEWRRPVDAVGKEIPRRAGVSSFGFGGVNAHVVLEEPAQTIRADMPEIPELIVLSARDREGLAASADALAKALTPYANTGGALEPTIESRLCACLADILEIDIDEVEPLTKLDDLGVEPVHRPLLRRSVEKVLGLTIDHDLVHRAGSIREISSAFQSLPEHSGMEAAPLLRDIAFTLRAGRDAMTERVAFAAQSLKELVDRLRILAATRDNLTGQDGFWHGRVPYKTRRHNKVTQSPKDVPLEELARLWVGGAAYDWEAERDGRDLRRVPLPGTSFKKERIWFDTLNGKPSAAVPQIKDTSLPSGMALTRKSDGVFEVSLSGDEFFLRDHIVQGQPVLPGVAYLELARSAGCLHLQSRDLALKDVVWVQPAVISEPQTLQVVLGSPGPGQEYPFRILSHGDSGERLHCRGAIAHLPEVPPEIINNDAIPSGRRIPSNEIYSLFETAGLHYGPGHQCLNWLVSDGSRVVADLSLPEICGSAVEPFVLHPSLMDGALQAAIGFGLAGEEQSGDLALPFAIESLQCLTANKSRMRVHLERRSVASAAHGIEKIDIALCDESGQVLTRINGFSTRRVALPEAGKTPAHQALGAQDPVNVPAQDGLKDAAAAYFKRLLSEALACPPDEIDLDEPLEYYGFDSHMVMELTAVLEKEFGTLSKTLFFEHQTLGAVLDHFIEAHGPSLTTVVRKGRGAAGTPASVDAAAKPRTEPKTGGLDIAVIGLAGRYPQAYDIAGFWDNLRNGRDGITEVPADRWKWQDYFSTDRSRIDAHFSKWGGFIDDVAAFDPLFFNISPGMAEAMDPQERLFLEHAWTAMEDAGYRPGDLQAQSVDEDGLPGQVGVYAGVMYGEYQLLGLQGSLAGEPMSTASYYAGVANRVSYALNLHGPSMAVDTMCSSSLTAIHLACADLALGRVRMAFAGGVNLNLHPNKYSLLSKGQFISSNGRCQSFGSEGDGYVPAEGVGVVLLKRLADAEADGDHIYGVIKGSALNHGGRANGYTVPNPEAQHHVIARALREAGVDPRAIGYVEAHGTGTKLGDPIEIKGLNDGYGPVLEGPCWIGSAKSNIGHGEAVSGLAGLTKVLLQLKAGEIAPSLHAETLNPNIDFAATPFRVNTSLRTWDAPVHEGKTLPRVSAVSSFGAGGSNAHLVVEEHCPPPSVEPYSYGPVLITLSAKAEDRLKAYACALADWAENAPAETSLRDLAYTLQVGREPMPHRIGVQVSTVEELARYLRQFLAGRDGPVRSGRARVVSNPTVENPDGLAAEVLLDGWMQGTVYDWRKIYGGEARRLSLPTYPFAREIYWPDTTAQPAPIALRTAATTAKTTETRALEAKSTGHTSVLHTDLLLLRPQWKDLPLTAPSIDPALRRVAHIGPMRNLQEHAQLALPASDPADPNTFTDQALALLRDLKELALQSSDQKVHYQVVLPASYSQSAALAGMLDSAARELPRLTCQVLCFDTDDPASGPLEADLKAVAAWPGKSRLRKKDGRWQALTWQEEQDVADAQPGGGWREGGRYLIVGGCGGLGAIVARHLAQTLSRVSLVLTGRSPSGPKQNALLQELRSKGAHADYLATDLGDAAAVRSMIRQTTDQGSLHGVIHCGGVLSDALILRKTEEDLRRVFAPKVTGVANLDRATDGLDLDLFLVFSSIAGTMGNPGQADYAAANAYLDQYVEERNRRALSPGGPRGMALSVAWPYWADGGMTLDAAAVNAMRDGAGLVPLSTARGLEALDRIVRAGWPQTMVLEGDGDRLAALIAAADAGQPAGAPAGPEPAPPPSSFHLQDAVEEYLAEELAKVLRISPQRLEADVPLVDYGVDSVAIMALTASIETVTGPLPATLFFENPTIEAAAGALTDLASQSLMEALHVPEPTVDLLEPAPGGTAEDQAPSEDPLLDNNAKPVRAEAAVPDTQSAGSGDIAIIAMEGRFPGAEDLEEFWDNLVNGRNSITEVPKDRWDAESLFDPDGAYEGKARCKWGGFLSDVDGFDARFFRITPDEAELLDPQERLFLETAWALMEKAGYMGPALRVDLESAVGVFAGSMTQQYHAVRSDPLREALTVLSSPSSIANRVSNVLDLNGPSLAVDTMCSSGIVAIHMACESLRAGACRAAIAGGVNVSIHPKKYIGLSASQFIGSRRDSTSFRDGDGYLPAEGVGAVLLRPLDDAVAAGDRVLALIKSTGINHSGRSNGYRVPSVAAQAKLIGDTIRQAGVPVNTITYAEAAANGAAMGDSIELAAFRQAFQDLTPEQKFCAIGSVKSNIGHAESASGLSQLAKVVLQMQAETLVPTLGTDALNPKLDFSSGPFRLQSELQAWARPIGSDAASGGSNQPLRAILNSVGAGGTNACMVLEEPPKTSAPPAAVAQDQYLIPLSARDEADLRVLAGRLKTYLETRPETRMADLALTLQTGRSQLDQRAAMISRDVPALLHQLEALAEGLEADGLVTGNTMTGQDALSGLLTGKTGAEIVSLLLRHRNLRKLAVAWVHGARLNWSPLQAEGAQRLALPAYPFRRTRYWLGGIDAREAVSQLEPDTRSDTTDPETCIRDYLINDLRIAPEEIDFRRSALDHGLNSVMLMPLCQALEARCGLTVGLGDIMESKSLATLLSRIAGKDGYAPMDNPKHAQPGTSDAVNTALPLTKGQIALWLHDQKTPGDAGYTVPMALRLAGSLDKDMLRAAFADLLKRHPVLTSVFTANGGMPQRIVQDGISYPIEELDLSGAPASVIENELHAFAGLPFDLTNGPLVRSLLIQEAADRHVLIICVHHIVFDGQSAMILIDDLMRLYEARLQGVRLPRPIGSSFDAFQRWQERLLTSERGTNIRAFWRDELEGHNELCLPGDWDADLECASKAGSHVLWIDKDTARRITEASTAHGATPAQFMMAAFVLILHRLTGSHDLLIGLPVLGRPDRSFDHTVGYFANLLPLRIRLSDQVSIRDLVRETRQTMLNALEHGDLPLSEMGEVSGTGRLLMPRVQFAFQSLVGPQNTDRGSLEVSVVDGIDQQGVQDLALEVYPGPEGMRCRFAYNARQFKSDTVSALADAYQKVLSTFLADPGGALVDVSLAGADDEVLTDWGHGGPPAPDEALIPAWRAQVRMAPDAPAVICGDTVLTNAALEQNAGDLAARLVDAGVQPGDVVASCLARSANSLVAVLATWWVGAVHMPLSPVQSSSRLDDMIADGAPVLALTDAKTASLLSIRQMRVDERTEISKATAGVLPTPVIQDPAAAAYILFTSGSSGRPKGVQVPHHALAHHIQAMANLFAVNDQDRVLQFVETSFDAAFEQWLTTLVRGATVVMRPEGLWSALDFAEAVQRWAVTVADLPPAFLDEVLRDLGRSDDWQLLQSLRTVVTGGEALTENTLSTWCDSPLADRALVNVYGPTETTIGSTAFVYRAQMDGPERRLPIGRPLPGENVFVLDVADQPLPAGLIGELAIGGVGLADGYIAAQNKQGGFSSGPGGKADRLYKTGDLARWRTDGQLEFLGRRDNQVNVRGFRVELAEVEAGLERIDGVLRAAVTVSDRKPDTTLQAYVTVSDPDLEPAAISRALKSSLPDYMWPSEIRVVTALPQTIAGKLDRQSLNGAPAPSVSIPEGPLSRIEKVLASLWAELLDCPSVPVTANIFELGAHSLLLIRFAGEIRSRLGAELSVAQLFQAPTVADQAVLIERAKGNRSSVVNLQAGSGPGLVLVHGGVGTLLCYRTLMKHLDPRFSILGLEMNRLDRWNSIPDAATAYLADLEFDQGQAPLHLAGWSSGGIVAWEMARQIERSGGELASLTLIDSYPPAVLSHIDNRIQPHDHEKALLAGFARDMGLAAELPSAEPKGAPEKYLQNMAENTGEDFQVLLTLFNNYKHIAKAVDGYTPEPVSVAASVFHAEGAEISSAMRGWPAEAGVLDIQPVPGGHLSMLEGEHSRFLANLLNGKLTTAHD

SEQ ID NO: 17 - protein, 437 aa, Labrenzia sp. PHM005
MTATRASALSVCVIGGGPLGIGLGRELSEGGIDYDLYEQESDLGGVWNTDAPCGRTYPSLHLISPKFNTQVPDFPMPDHYPAYPNHKMMLDYIRSYARHFGVYDHAHCNTGVTWIEPDGDGWNVELSTGATRRYDIVAVCNGAQRVPHYPKPPYPGTFSGEVLHTADYKNPSQIAGKRVLVIGAGNSGCDVAVDAVHHAVSVHHSTRRGYHYYPKFIDGKPTPQWMLQLGTKFTSKEETSAYIQKVFKLAGFDGTDFGLPAPDHPIDAAHPIMNSQILYHIGHGDIATVGDVAGFDDLTVRFKDGHEAEIDIIVYATGYDRHFPFIDPDILDWKDGIPDLFIHIVPRNLNNLFFFGFVNAAAGLGDGMRLQGQFVRSYVRAFENQTLGYQKFVAAKAQDDPDLGQDYFVDSRRHTWEVDFWKFIRHARYYREMLDDD

SEQ ID NO: 18 - protein, 2764 aa, Labrenzia sp. PHM005
MKDHSGIVPVAFFLDRLLDLEGDGALCNIVFPQPLRINEGRATALLQQTGGRLEITLDGVRYCQADHEKGSDTAFTRPRPVDLDARRTETPFVLTSRACDAVLQSTHGPSLMSLAEQRNGPSGALARVQSAEMGARRRVAVLNGALLAAVVWCQTQREESTLPMPYGIGSLTQFTPTLPDKVLVDLRPARKGPPGADRVTLDLDLCDDNGSVFLALRGLELVWSEKQQLPGPNQLLFAGPCWQEISPPLMNGTAPVDPVLFVTQTDAHRQSTLRAAFPGADLRVLSDTVENAFAEILKFVQSNDPVRGARPVLLIVPDQSLASSLSGFMRCLRLEHPASCAQAVLVPGSLSDRALTSGLKQVLNSGQLPMLSRLTESGPQNGWVREIPLPSRTAYFAAGDVIWITGGLGGIGRILARHYASAGQRVVLTGRSAPPSGAEAFLTETGALYLQGDVTDCSTATLLARDILAKHGRLDAVIHAAGLIEDGLLRDKGQESAARVLAPKLAGTRALDEATAELPLKAFVLCSSVAGVLGNVGQADYACANAYLDVFAELRQGQVLNGQRHGQSLSVAWPLWQGGGMAMTDENARMMRTGTGMVPMPDGTGIEALERALASGETRLVVAYGLPEEIRERFLGFEYPAGNNVLEPAAVEQQADQSELETRLRDLVAKVQHIPVQKVTRYKPLSDYGFDSISFTELANEVNSAFGLRLMPTVFFEIPDLAALADKLAKDHSVTLEPEKRPSSVTSPAPARAVVDQEKPVRSSAGFDGSVSIGKAPSVNRGMDTAEPIAVIGMAAKLPGVQSLDAFWRALDAGRDLISEVPADRWDWRAFQSGPDEDKSALKWGGFLADMACFDHAHFGISPAEAEVLDPQLRLMLETLWATLENAGVAPDSVSGSRTGVFTGVATCDYSELLAKAREAGHLRSAAEPFSFLVANRASYFFNLHGPSETIDTACSSSLIAIHRATESLRAGMCDMALAGGVNILATPRITLASSRAGMLSEDGRCMSFDARANGYVRSEGVGAVLLKPLADAQRDGDRVLGVIRASGENHGGRASSPTAPNATAQKELIVDVVRRAGIDPASIGYFEAHGTGTELGDPVEVNGLKAALSELGLDARDGPIWLGSVKANVGHTEAAAGVVSLIKLLLMLRHNRIAGNPHLRDPNPYLDLDETPLSLVRGSLDWPSGPAPRRAGLSSFGVGGSNAHLVLEEPATDTEPGLPGSSPAEAEIIILSARTPEIRAQMAGDLAQHLRANQDTLCLSDVAHTLRVGRARLPARLAFETSSLTETIQLLETVAQGQVPENVTLGETEEITGIALSEDLPDLIEVWLAKGQLSRVLKAWVAGADLDWAQVAPKREGRRIELPGYPFERITHWIGSESPEALHVPDAAAALPSVRQFAEEWEPSPLLEPGSGPVGRVLVLAPKSMSAADADLNAGEDLLWLTPEPEDLQNSEAAARLLSWLEPASHVLLLLGDEDRVAGPIIHLLQALAQGRQRPQSLMICGHAETPEDLAWLDALVGVQRSCRSALPDLNVSIVFGSGTSLTVMVRHALAEMTAGAGVCVRYRGEERQICASRALKAPPDVQTPWRHRGVYWIVGGSGAVGSVLARHLARTVSARLVLSGRGPEDRALIDELCALGADVCYLPADVTDIAALHTVRDQIFSRWDRLDGAFHLAGRSGAAPLIEAKASGFDSVLAPKLQGTKNLHEVLTNSGADFLCLFSSSSAVLGDLGSGDYAMANRFQSAFAAEHNNETLPVLAVEWPLWRARGLADAESESLYLASSGQVPLEGEQAMQALETAVFTGRTRTLVLSGNAERLDHLAGTPQKSKPSAETGDVLTVLKSLAADQLKMSSGEIGSHKNLASFGFDSIALSEFARSIGTCFDIDLAPSVFFSHATLGKLAAHLSEIGVGVTTPESTQPRTFAQPRAVSDDAIAIIGTSGRFPGARDVGGLWNILDQGREAVEEVTPERFDWRRIYEAKTPPVPGKTNSRWCGQVPGLSEFDPLFFEISPLEAERMDPRQRHLLQESWLALESAALGPEHLASQRVGSFVGVEDGSDYIKRSDQISLTGAHNAVLAARLSYFLGLDGPALALNTACSSGLMAAHMACQSLRAGECDVALAAGVNLMVSQDAYIGMGQAGMLSPDGKCYTFDVRANGMVPGEAVAVLVLKSLARAREDGDPIQAVIRTSGTNYDGHTNGITAPSGQSQVDLLRRVQAQAGVKPHEIDWVIAHGTGTELGDLVEAHALRDVFSGAEREPNSIAVTTTKGNFGHTFAASGLVSAIGAVHALQHDRLPASLNHNQPSPMLGWQKTPLYVNTQSRDWPRPHAGRSRLISVSAFGISGTNVNLLIEDAPDSPAQLPSEERNYVISLSAKTESSLQAMASKLAAYLKSPEAADQQLAAISLTLLTGRHAFTHRLALVVKDLQDAARQLEAFDSTPGYRGHVPEEPDLPDMSQQISGLLEKAQSREALHELAELFCQGHPIPWVNLFPCSLRRINLPGYVFERDRCWIDAPEARPAPAIGPYVKPLPEPDTPAHPPVSGVSDLSPGLDMLEAARGAASNVLNRDVQTLSRIVWGAPQSSEIRPDPNEICILSADQGLVAVEAAGTTDALALLAQAGAPCSSFPAPVRLPRLRGGLKPVSAPQGVAALYGDEGRLVGNMKGLSAPAVFDVRVLRAIWNSVQCLSDLETAQVAWPASLMTLASTAPLTSDVHFEVVRLSDPDPGYLNVDVTVYDPQGTPLMILREFSLSLGALPENIQWEGVEA

SEQ ID NO: 19 - protein, 1949 aa, Labrenzia sp. PHM005
MPDLRDIALTLQTGREAMAERAAFLVQDHQDLLTQLRIVEDGGIPDKGARGRVNLSETGPREEAIGSSRLRSQNNGTLDEIVQAWVSGQEIDWSSLAGMAGARRIGLPLYPFDTHRLWFDEVVTEDNAENPNAPDPVPEHVTFSPYWESVSPTDKPAPLIGPVLAIGATGASRDQLANAYPDAQFVPPDEAPKKLRENWGTVLWLAEPGAAPLTFFRFAKALIETGPASGNLTLVTRNGFAFDAEPADPEQAAIQGCLAVLAQELPGWTLRAMDLHPAEPLFPNLLDTLPLEGGQIGFARRQGQWLRPRLIPCDLPEVPPEIPYRKNGVYLVLGGAGALGRVWTTHLLQRVSAQVVWLGRSALSAQIRQNMAAYDGAVSYHSADARNPGELADAIADIRNRYEKLDGVIVSTLAEYDKSIAEMSETLFQDILSTRLNVVSALDKALMGVPTPDFVALFSSLASCGKPAGMAAYVAGCQASEAAAFALGRSHSCPVTVVNWGYWDIGGGVRVTDSLRALAARRGVVPIDPEAGMALFETALAMKQPQIAISRTTRPDRIETVLETPRMKPLSGTALPVLPQVVTREAPPEPAREAAALDQWLGRLLLAQLRKMDVFDRPGLSRKIEFETFAILAKFRPWWDEALNILEEQGSISRDAAGAVTLLGDDLLSPDTVWAEWEKAQQAFLETPDTRVLAILTTDCLKALPQILRGQALVTDILFPAGKMEKIEGLYSNNRICDFFNSVVADTVDAVITARRAQDPEAKLRILEIGAGTGGTTATLVPRLARWSEAIAEYCYTDLSKSFFTHARRRFGQSAPYMRFELFNVEAAPAAQGLDIGAYDIVLGTNVLHATRDIRETVRNAKALLKSGGVLIANDISDKTVFASVLFGLIDGWSLAEDRHFRIPGSPGLYPETWETVFALEGLQHVQFPAEAQHGLGQQIVVGQSDGRVAVSEPFEVEVVHPGPLEHGTTDDNSVSEEEIHSGTQVRGRGLLSNEAIRAEIEDALAAALDIDRDEIASDVPFSDYGVDSILGVGFVREIGARLSITLQTTDLFDHTTVARLCSFIEEQHHPAVGGAMSETDIEPKVTTDPQRKLERWDDGIAVIGMAGQFPGAADVDTLWRNMIDGVDPVVPLPGRYMRPEKVSQDKEPGKSYCRWGGILEDRDAFDPLFFRLSPREAASMNPHQRLILLESWHALEDAGIDPGGLAESRTGVFVGCEPSGYVHDTFTGASDAIVASRISYFLDLKGPAYVVNTGCSSSGVALHLACESLRNGECDLALAGGAFAVMGENILIGLAQTEMLTRTGHCRTFDAEADGMVMSEAAGMVVLKPLSAAVHDGDPIHGVIRASGTNQDGASNGITAPSGAAQAALISDVQSRFDIDPRRISYVETHGTGTKLGDPVEANALVKAFQPHDLTPGSCALGSVKSHIGHSAAAAGVCGLIAVLMAMKHRKMPELRHFKSLNPLINLEGAPFYPLTETSDWTRRDGQPLLAALNSFGHSGTNAHLVIEEAPELRVSPTVSVGDPQQELILLSAKDVERLQLQAGALARKIENVPDLLLADIAHTLRTGRMAMECRAAFLVTTRTELLDRFKGLAAGTLAADWSGEVPSKWTARAGPQPEAPSSTAVLSMQAEAWVAGAPIDWSGVALHQGWRGQRCHLPGYPFAKERYWRSDRQDQDRDKSGHDTLHLNGEESWLRDHRIAGRPVVPGVAYPALALARLTGARNTGWRFEDLVWPVPLTVEAPVDLEIEAKSFDQDGSYALSSLAPDGTSQVHHQGRLIPLEGPPPAVDLPSIRARLSAHEMAVDAIYGALNEAGVVHGPALKSIGRVWATPDEILAELNLPGTAESGVMPIALLDGAWQATLALSLADPNNPAPAALPFSLETLDLHAPLGRVRFWSRRNGARAWWTCKFCCPMGHQRCKCAGCTPGPSALPNPRLLKSHWMRRTRF

SEQ ID NO: 20 - protein, 875 aa, Labrenzia sp. PHM005
MAGALRSEANFDGPLHRQLTEGAPLTPVWHAQTLFTLEGQSPWRTGGVYVLSGGAGGIGLHLARHIAHAAEGARLILLARSAIDPERLASLRHTGCDATVIRCDLGNPGEVNSAIQQVLKKFGALHGVLHLAGVNGDGLLASDLERQCDAMLAPKVIGARALDQATAGLDLDLFVMASSVATLRGSPGQAAYCLANGFLDSFARKRAQAVAAGERFGQSLALHWPLWDDGGMRPPDADTEMAMRQNTGLCPIPAGIALKALDSALQQGLTEAAVFYGNQDKALSWLSSDAGGPKQSGPQNTVGDLPQRLEHRLKALIGPILGRDAEALNPVEPLQHYGIDSITITRIARDLQSLAGPGAQTLLFRFSTIRSLAEHLAKTYGAACHEWIKEAAAITPQNSNTTSVRPTGTTQLSATESISSPAHARAEKSESIAIIGLAGRYPGSDSLEGFWQNLAQGRDCITEIPEERWRLDGFFEPDETRAVAQGKSYSKWGGFLEGFADFDPLFFNMSPREARDIDPQERIFLQCVWHALEDAALTRKDLKEHYDQNVGVFAGVTKTGFDLYGPAQRAAGKVAFPHTSFGSIANRVSYVLDLHGPSMPIDTMCSSGLTAIHQACAALLDRSTNLAIAGAVNLYLHSSNYAELCSAYMLSRSGRCRSFGADADGYVPGEGVGAAVLKRLSEAEQDGDRIHGVIRSTAVNHGGHTHGYTVPNPRAQAALVRSALNKAGIDADTIGYVEAHGTGTPLGDPIEVDGLVEAFASGNVLPGQCWLGSVKSNVGHLEAAAGLAGLTKVLMQMRAGQIAPSLHADAVNPAIDFGNTPFRVPTVLTEWTPADDKPRRAGISSFGPAEPMHMWWSKNIRRHLPNRARLNPGQF

SEQ ID NO: 21 - protein, 2142 aa, Labrenzia sp. PHM005
MEAASGLAALLKVVHSFAADRIFGIAGFDQVHPEIREDGAACALAVNDTPWPRSGTPRHAGIHCHAMSGVNAHILLQEPPCKSVARPQDAPADPQVIVLSAASPSSLERMIANLLKHLQQQPERLCDVAKTLQQGRDALAYRIAWVVPDTAALIEALEVETRGQATSDWPVFRGTAGSGIQAEETVTGLEALARAWVTGVDQSWPDLEDQSARRIRLPGYAFDCRPHWVKPVLERAPDTSAQIGIKPFLIDQIAGVLDLPAASIDTKQHLYDFGVDSLFAMQLLRAVARTFGITVRGRDLMEHQSIDALAEYYTTQLPALAVDPEPQAVEVCEDRGHARDLPLSQGQAGLWAIAQAQPGTSAYNLPVCLHSREGFDTTAVQSALNKCLVQYPVLTSTFRVGRRGPLRDENHGATLYVRQLDLPQEDPLATLRHAAKSPFDLARDLPVRATIFGQQGTPSYLLITFHHIVFDGGSFWLFMQTFLDAYDAELGKSLRAEATILPNKGADQAAFVATAKAAASGSEMRDARAFWARRLEGQLPCLSLTPDKPRNTARLFEGAHLTLPLPASVAGAMRSYSRAERCPLSSLCLALFATLLHRLSGDDDIIVGMPDHGRHDPRYAETVGYLVNMLPIRMQGLAGRTLRDLAYHLQGEVADALDHAAYPFAQMVRDLGLSSGPGEPPVFRVAFEYQNAFSHDALPALHQRLQVTGDLTLVEDLRQEGEYELVLEVRETSDTLSLCMKYNPDLYSEQRVQGWLEALTNLAQQALADPEANLDSFDIVGTSDRAKLLAWGTGPKPEFSADTVMQLVQRQTDMHSAETAVVDCDGAWTYEQLDQESLRVAAAIQQAGVRPGDRVALCLGRRRNYSAALLGTLRAGAVFVPLDPAHPKARLRHILEDCAPRAILADVSTDAMATQLAEPDCTMVRVDALSCAPEPQPVGLKGGDPAYLIYTSGSTGRPKGVQVPHRALANFLQAMAQRPGAGTGDRLLAVTTFAFDISLLELLLPITSGGSVHICPEEIAQDPDALASEISRVKPDILQATASVWTMLFAAGWQPPDGLKALCGGEPMPDRLNSLFQNSKLDAWNMYGPTETTIWSTCGPVTGSQDTVTIGMPIAFTEVLVLDEYLQLVPVGEQGELYISGAGLADGYWQQADRTAQSFIAHPYRSGERLYKTGDLASWSPSGGLIHHGRRDQQIKLRGHRIELAEIECVLDRHKELRESAVVLRKSGPEAQLVAYVVPEREAVPAVELRACLREDLPAYMLPDLIISLANLPLTPAGKIDRMALAARQVDLGHDRSASPEIEPGPPDMDLEKEVLALWSDVLDSTGIGRDIGFFEAGGNSVTAAVLAARISERFGVELRVSDLFRFPTIRAQARHLGAGTSDVVPASQKQVTAAHEAPKLNHFAAPSLAQRLDDEPLAVIGLSCAVPGALDLQSFWQNLLDGREAREVLTPEELRAAGVPDAQLSQPDFVPVAFPLAERACFDPGFFNISARAALHMDPQSRLLLQHAWKAMEEAGHSTASLPKTAVFTAVSHGHYKTLLHDCQAVSDDEFYSAWIAGQGGTVPTMLSYQLGLTGPSMAVHSNCSSGLVALHQARQALLAGEARAALIGAASVYAVPGAGYLHQPGLNVSSDGHCRAFDAKADGLVAGEGLGVVLVKRLSDAQADGDHIHALIKGVGISNDGADKAGFFAPSVQGQSEAIRRALESAKVDPASIGYIEAHGTGTRLGDPVEILGLQSVYGRAAGAPQPVRIGSLKPNIGHLDTAAGLVGLIKAVMAVKTGEIPPSINFETPNPEIDFEDAGLEVAAIRQGWPETSGSPRRAGISAFGIGGTNAHAIVEEFQPESAMPVSPVAEPSSQIVPVSARTQDGLRQLLSRLLAVVEDKAEAPLADIAYTLQTGRRHMVYRKAFVVSGLDELRAELKACLSTAELLEDQPAASMPKLKSQEMSVLMEHWLATRQLDRVAEAWTGGTEVDWTQLHTGPRRRVSLPTYPFAKEIFWPGKPGAQPSAGSMQSLLLTQDRQVANRIPVSAPAGVQKVWLMGALGQHQQTLSELLPDARITDLPGESGADPASHYMKLSRALLAKARDLALEGGAGLLQIVLDARGPGVPVFLPPWRRRSRTCAFKSYKSLRPYRFRTWLAH

SEQ ID NO: 22 - protein, 377 aa, Labrenzia sp. PHM005
MNSDEAWNEIEAAILASMQCQDKFSNTPPQDHDGAAREPAPIAIVGASGMLPGCEDLKAFYAALETGACLIEKRAERSLGERLSAPAADAPFVYGGFVPDPAGFDAGFFDIPKSEADQMDPRQRLLLMAALGAMYDAGYASRNLRGSRTGVFVAAQDNEYDRLCASLGHDPDAGYAQSCLLANRLSYFYDFDGPSEVIEAQCASAGVALHRAVQALRQGEISQALVAGVNLMLTPGPFRHLAETGQLSLDGKVSPFGATAAGHVRAEAALCVVLKPLSEAVADGDSVYAVIRQTSVNFNGRGAASLAAPSVTRHAELIADCYRSVGIGPGQVGVIEAQGMGNPLSDIAEWESFNRAMKRFGQEAGAAALMRSVSSVR

SEQ ID NO: 23 - protein, 278 aa, Labrenzia sp. PHM005
MSRSTLETTGASNDTVEDHYDSPALRLGPILFDEHLHWGYWDEDSRDASFGAAAEAMCHRMIDRTEIGPGERFVDLGCGIGHPALKLAQARSCHVTGVTISGYQHRIAGEKAAQAGFSDRLDFLQADARSVPLPDKSFDGGWFFESIFHMGHAEALGEAARLLKPGAGLVLTDLPTLPHTTPEFMDFVHEHIHSVFVPEDRYPALMADAGFELLNIEDISENVMPWLETKLREAVQEKWSDVVRLMGDQAEKAVDNWYYLFEYMAENLGYTMITARRL

(19) United States
(12) Patent Application Publication
(10) Pub. No.: US 2021/0317490 A1
(43) Pub. Date: Oct. 14, 2021

(54) ANTICANCER COMPOUNDS

(71) Applicant: Pharma Mar, S.A., Colmenar Viejo - Madrid (ES)

(72) Inventors: Librada María CAÑEDO HERNÁNDEZ, Madrid (ES); Fernando DE LA CALLE VERDÚ, Madrid (ES); María Pilar RODRÍGUEZ RAMOS, Madrid (ES); María del Carmen SCHLEISSNER SÁNCHEZ, Madrid (ES); Paz ZÚÑIGA GIRÓN, Madrid (ES)

(21) Appl. No.: 16/494,720

(22) PCT Filed: Mar. 16, 2018
(86) PCT No.: PCT/EP2018/056665

§ 371 Date: Sep. 16, 2019

(30) Foreign Application Priority Data
Mar. 17, 2017 (EP) 17382140.6

(51) Int. Cl. C12P 17/06; C12N 1/20; C07D 407/12

(52) U.S. Cl. CPC C12P 17/06; C12R 2001/38; C07D 407/12; C12N 1/205

(57) ABSTRACT

Anticancer compounds of general formula I

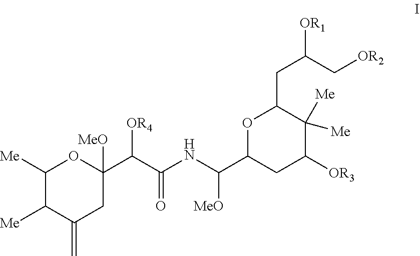

wherein R₁ to R₄ take various meanings, for use in the treatment of cancer. A novel Labrenzia sp. strain named PHM005 with Accession Deposit Number CECT-9225, a method of producing compounds of the invention and analogues thereof by using the PHM005 strain and the Lab gene cluster codifying the biosynthesis of pederin-like and onnamide-like compounds are also provided.

### SEQUENCE LISTING

The content of the ASCII text file of the sequence listing named “16_494720”, which is 271 kB in size and was created and electronically submitted via EFS-Web on Aug. 12, 2020, is incorporated by reference in its entirety.

### FIELD OF THE INVENTION

The present invention relates to direct or indirect production of anticancer compounds from bacteria and to new anticancer compounds, pharmaceutical compositions comprising them and their use as anticancer agents.

### BACKGROUND OF THE INVENTION

In 1949, Ueta reported the isolation of the toxic principle from the beetle Paederus fuscipes (Kyushu Igaku Zasshi, 1949, 249). Four years later, a substance with identical physical properties from the same beetle species was also described by Pavan and Bo (Physiol. Comp. Oecol. 1953, 3, 307). The structure of this toxic compound, named pederin, was first proposed in 1965 by Cardani and co-workers (Tetrahedron Lett. 1965, 2537) but it was corrected in 1968 by Furusaki and co-workers based upon the crystal structure of a derivative. (Tetrahedron Lett. 1968, 6301). The structure of pederin is:

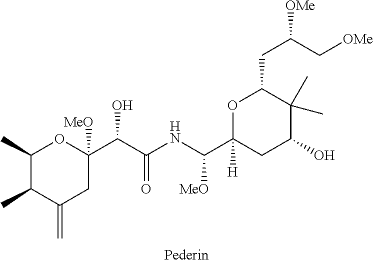

Additionally, Cardani's group has reported the isolation of two additional compounds from Paederus fuscipes that were named pseudopederin and pederone. Pederone was described two years later (Tetrahedron Lett. 1967, 41, 4023).

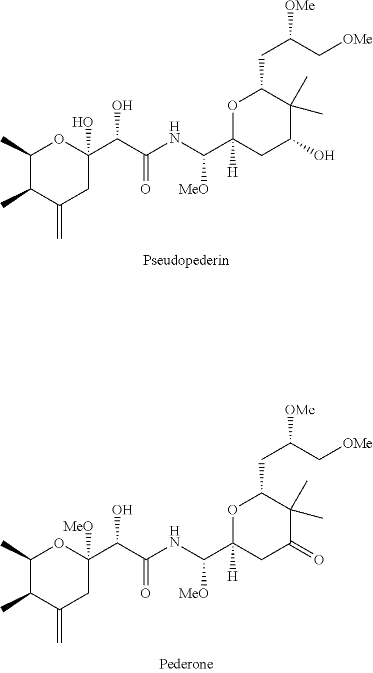 [SMILES: C=C1C[C@](O)([C@H](O)C(=O)N[C@@H](OC)[C@@H]2C[C@@H](O)C(C)(C)[C@@H](C[C@@H](COC)OC)O2)O[C@H](C)[C@@H]1C ; C=C1C[C@](OC)([C@H](O)C(=O)N[C@@H](OC)[C@@H]2CC(=O)C(C)(C)[C@@H](C[C@@H](COC)OC)O2)O[C@H](C)[C@@H]1C]

Pederin is a potent cytotoxic and vesicant agent. Brega and co-workers (J. Cell Biol. 1968, 485-496) have tested pederin against a number of cell lines such as EUE, E6D, HeLa, KB, Hep, AS, MEF, CE, BHK, Z1 and M1 and have reported that concentrations of the order of 3 nM are sufficient to cause cellular death within four days in all the cell lines analyzed. In addition pederin causes an immediate impairment of protein and DNA synthesis.

The cytotoxicity of pseudopederin has also been described by Soldati and co-workers (Experientia 1966, 3, 176-178). Pseudopederin has toxicity lower than pederin, being active at doses 10 times higher.

European patent EP0289203 discloses the isolation and antitumoral and antiviral activity of Mycalamide A, a compound isolated from Mycale sp. sponges collected in New Zealand.

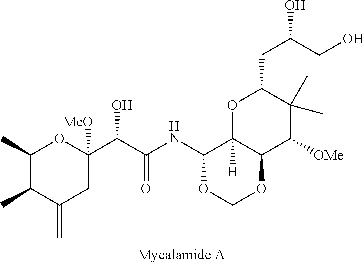

Its inventors, the Munro's group, further reported the isolation of Mycalamide B, a closely related compound with antitumoral and antiviral activity, from the same source (J. Org. Chem. 1990, 55, 223).

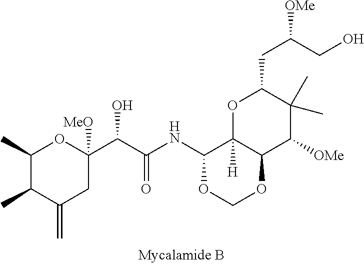

They further isolated two additional mycalamides, Mycalamides C and D, from Stylinos sponges (J. Nat. Prod. 2000, 63, 704). Mycalamides A, B, C and D have IC₅₀ values against the P-388 murine leukemia cell line of 3.0, 0.7, 95.0 and 35 ng/mL, respectively.

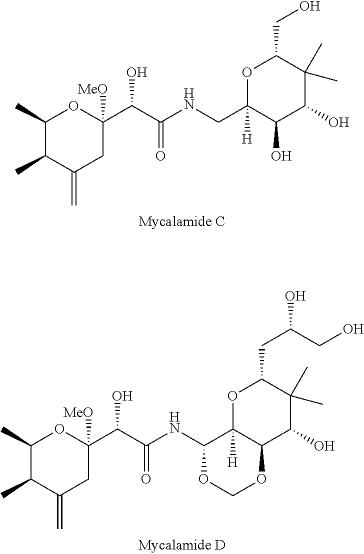

Mycalamides have also been shown to be powerful immunosuppressive agents with comparable in vitro efficacy to the clinical agent cyclosporine A.

U.S. Pat. No. 4,801,606 describes the isolation of Onnamide A from Theonella sp. samples collected off the coast of Japan. Onnamide A is an antitumoral compound with an IC₅₀ value against the murine P388 cell line of 1 ng/mL. It also has antiviral activity.

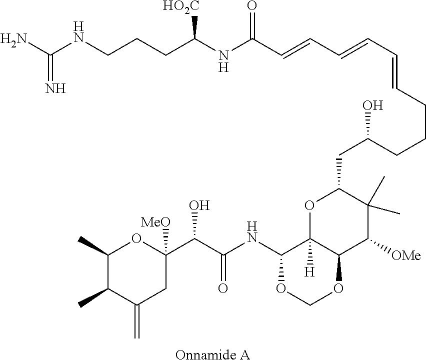

The onnamide family contains several members. Three of them, Onnamides D-F, lack of the dioxolane ring of onnamide A. Onnamides D and E were isolated from Theonella sponges by Matsunaga and co-workers (Tetrahedron, 1992, 48, 8369) and Onnamide F was collected by the Capon group from the sponge Trachycladus laevispirulifer (J. Nat. Prod. 2001, 64, 640).

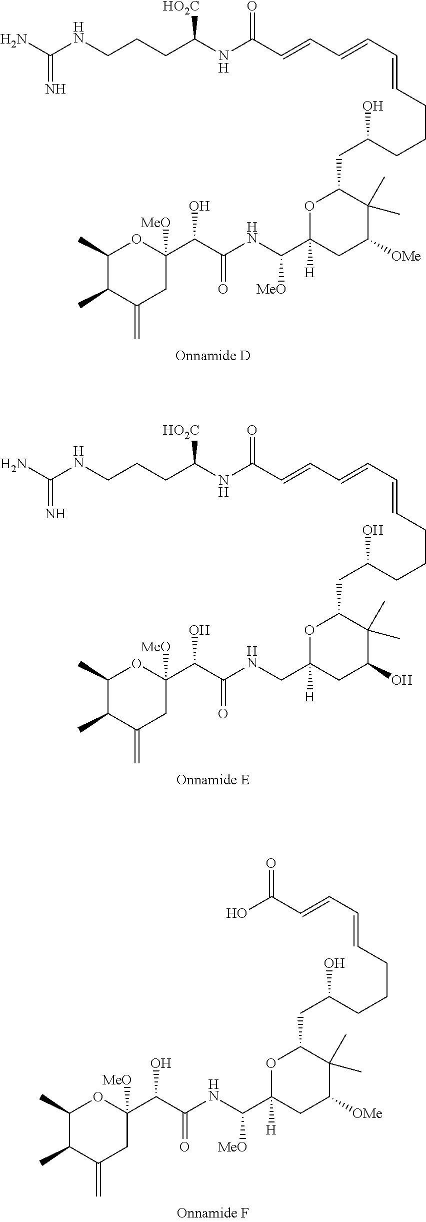 [SMILES: C=C1C[C@](OC)([C@H](O)C(=O)N[C@@H](OC)[C@@H]2C[C@@H](OC)C(C)(C)[C@@H](C[C@H](O)CCC/C=C/C=C/C=C/C(=O)N[C@@H](CCCNC(=N)N)C(=O)O)O2)O[C@H](C)[C@@H]1C ; C=C1C[C@](OC)([C@H](O)C(=O)NC[C@@H]2C[C@H](O)C(C)(C)[C@@H](C[C@H](O)CCC/C=C/C=C/C=C/C(=O)N[C@@H](CCCNC(=N)N)C(=O)O)O2)O[C@H](C)[C@@H]1C ; C=C1C[C@](OC)([C@H](O)C(=O)N[C@@H](OC)[C@@H]2C[C@@H](OC)C(C)(C)[C@@H](C[C@H](O)CCC/C=C/C=C/C(=O)O)O2)O[C@H](C)[C@@H]1C]

Onnamide E did not show cytotoxic activity against the P388 cell line at a concentration of 0.4 μg/mL and Onnamide F has been described as a potent nematocide.

Experimental evidence for a bacterial biosynthesis of pederin was first provided by Kellner, who reported that the pederin-producing trait could be transferred to nonproducing Paederus spp. lines by feeding eggs of pederin-positive females (Chemoecology, 2001, 11, 127). In contrast, eggs treated with antibiotics did not cause this effect. This result indicated the existence of a pederin-producing bacterium that is able to colonize the nonproducers (J. Insect. Physiol., 2001, 47, 475).

Piel and co-workers isolated the gene cluster for the polyketide synthase (PKS) of pederin (Proc. Natl. Acad. Sci. USA., 2002, 99, 14002 and WO2003044186), and onnamides (Proc. Natl. Acad. Sci. USA., 2004, 101, 16222). This work strongly implicated bacterial symbionts as the true sources of these compounds, which provides an explanation for the isolation of structurally similar compounds from disparate organisms. For a review about the symbiont proposal see Piel, J., Curr. Med. Chem. 2006, 13, 39.

Another closely related compound, diaphorin, was isolated from the insect Diaphorina citri by Nakabachi and co-workers (Current Biology 2013, 23(15), 1478-1484). This compound is also cytotoxic with an IC₅₀ value of ca. 1 μM and ca. 2 μM against B104 and HeLa cells, respectively. Its presence in extracts of Diaphorina citri was predicted in the same publication by the analysis of the polyketide synthase (PKS) system of Candidatus Profftella armatura, a defensive bacterial symbiont associated with Diaphorina citri.

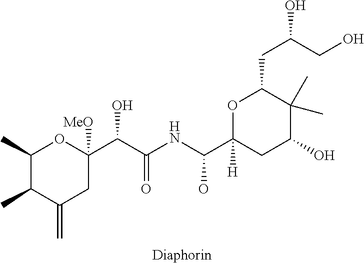 [SMILES: C=C1C[C@](OC)([C@H](O)C(=O)N[C@@H](O)[C@@H]2C[C@@H](O)C(C)(C)[C@@H](C[C@H](O)CO)O2)O[C@H](C)[C@@H]1C]

On the other hand, patent application WO2013016120 describes a total synthesis of pederin and analogues thereof of formula:

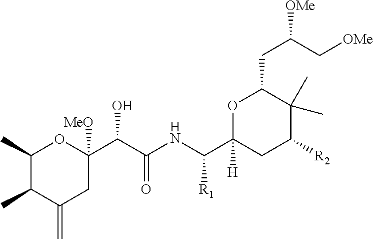

wherein at least one of R₁ or R₂ includes a linker that includes a reactive functional group that can bind to a targeting moiety. This total synthesis is based on a multicomponent acyl aminal construction.

Detailed studies on the pharmacological properties of pederins, mycalamides and onnamides have been hampered by the scarcity of these compounds from natural sources. For example, approximately 100 kg of Paederus fuscipes were required to isolate sufficient material to elucidate the structure of pederin. Although the PKS systems of pederins and onnamides have been described, it has not yet been possible to obtain these compounds by biotechnological methods. Therefore, the only practical way to obtain these interesting compounds at the moment is total synthesis. A number of total syntheses of pederin and mycalamides have been reported. They have been recently reviewed by Witezak and co-workers (Mini Rev. Med. Chem. 2012, 12(14), 1520-1532) and by Floreancig and Mosey (Nat. Prod. Rep. 2012, 29, 980).

These syntheses have led to routes that are sufficiently brief to deliver sufficient material for biological testing and have provided analogues that have been useful in developing structure-activity relationships for these compounds. However, the need remains to provide a more concise route to these compounds and new antitumoral analogues thereof.

### SUMMARY OF THE INVENTION

In a first aspect, the present invention is directed to a compound of general formula I or a pharmaceutically acceptable salt, tautomer, or stereoisomer thereof.

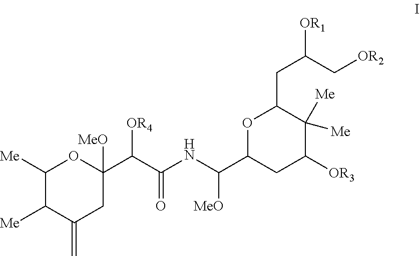

wherein:
R₁, R₂, and R₃ are each independently selected from hydrogen, substituted or unsubstituted C₁-C₁₂ alkyl, substituted or unsubstituted C₂-C₁₂ alkenyl, substituted or unsubstituted C₂-C₁₂ alkynyl, —C(═O)Rₐ, —C(═O)OR_{b} and —(C═O)NR_{c}R_{d};
R₄ is selected from hydrogen, —C(═O)Rₐ, —C(═O)OR_{b}, and —C(═O)NR_{c}R_{d};
Rₐ is selected from hydrogen, substituted or unsubstituted C₁-C₁₂ alkyl, substituted or unsubstituted C₂-C₁₂ alkenyl, substituted or unsubstituted C₂-C₁₂ alkynyl, aryl, and heterocyclyl;
R_{b} is selected from substituted or unsubstituted C₁-C₁₂ alkyl, substituted or unsubstituted C₂-C₁₂ alkenyl, substituted or unsubstituted C₂-C₁₂ alkynyl, aryl, and heterocyclyl;
R_{c} and R_{d} are independently selected from hydrogen, substituted or unsubstituted C₁-C₁₂ alkyl, substituted or unsubstituted C₂-C₁₂ alkenyl, substituted or unsubstituted C₂-C₁₂ alkynyl, aryl and heterocyclyl;
with the proviso that R₁ and R₂ are not simultaneously methyl.

In a second aspect, the present invention is directed to pharmaceutical compositions comprising a compound of formula I, or a pharmaceutically acceptable salt, tautomer, or stereoisomer thereof, together with a pharmaceutically acceptable carrier or diluent.

In a third aspect, the present invention is directed to compounds of formula I, or pharmaceutically acceptable salts, tautomers, or stereoisomers thereof, for use as a medicament, in particular as a medicament for treating cancer.

In a fourth aspect, the present invention is directed to pharmaceutical compositions comprising a compound of formula I, for use as a medicament, in particular as a medicament for treating cancer.

In a fifth aspect, the present invention is also directed to the use of a compound of formula I, or a pharmaceutically acceptable salt, tautomer, or stereoisomer thereof, in the treatment of cancer, or in the preparation of a medicament, preferably for the treatment of cancer. Other aspects of the invention are methods of treatment, and compounds for use in these methods. Therefore, the present invention further provides a method of treating a patient, notably a human affected by cancer which comprises administering to said affected individual in need thereof a therapeutically effective amount of a compound as defined above.

In a sixth aspect, the present invention is directed to a process for obtaining a compound of formula II or a pharmaceutically acceptable salt, tautomer, or stereoisomer thereof.

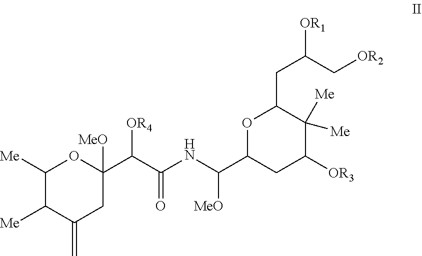

wherein

R₁, R₂, and R₃ are each independently selected from hydrogen, substituted or unsubstituted C₁-C₁₂ alkyl, substituted or unsubstituted C₂-C₁₂ alkenyl, substituted or unsubstituted C₂-C₁₂ alkynyl, C(═O)Rₐ, —C(═O)OR_{b} and —(C═O)NR_{c}R_{d};
R₄ is selected from hydrogen, —C(═O)Rₐ, —C(═O)OR_{b}, and —C(═O)NR_{c}R_{d};
Rₐ is selected from hydrogen, substituted or unsubstituted C₁-C₁₂ alkyl, substituted or unsubstituted C₂-C₁₂ alkenyl, substituted or unsubstituted C₂-C₁₂ alkynyl, aryl, and heterocyclyl;
R_{b} is selected from substituted or unsubstituted C₁-C₁₂ alkyl, substituted or unsubstituted C₂-C₁₂ alkenyl, substituted or unsubstituted C₂-C₁₂ alkynyl, aryl, and heterocyclyl;
R_{c} and R_{d} are independently selected from hydrogen, substituted or unsubstituted C₁-C₁₂ alkyl, substituted or unsubstituted C₂-C₁₂ alkenyl, substituted or unsubstituted C₂-C₁₂ alkynyl, aryl and heterocyclyl;

the process comprising the steps of:
culturing the wild type marine bacteria strain PHM005 or their mutants under suitable conditions to produce compounds 1 and/or 2 of formula:

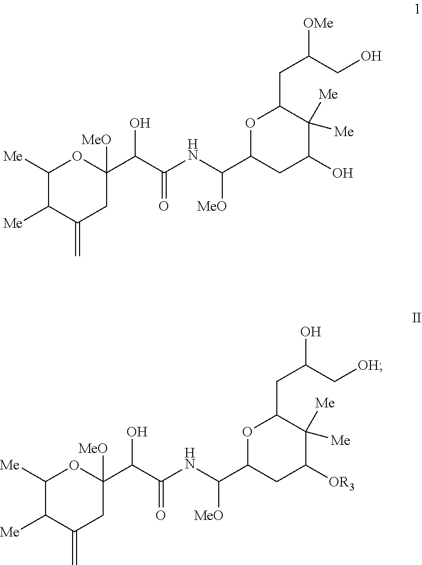

isolating compound 1 or 2; and, if needed,
derivatizing compound 1 or 2.

In a seventh aspect, the present invention relates to strain PHM005. The free-living marine alphaproteobacteria producer of compounds 1 and 2 has been deposited for patent purposes in the CECT collection with the code CECT-9225.

In an eighth aspect, the present invention provides an isolated nucleic acid sequence comprising the Lab biosynthetic gene cluster or being complementary to a sequence comprising the Lab biosynthetic gene cluster. This gene cluster represents the first example of genes from a cultivable bacterium encoding the biosynthesis of pederin-like and onnamide-like compounds.

In a nineth aspect, the present invention provides nucleic acid fragments selected from the group consisting of genes lab706, lab707, lab708, lab709, lab710, lab711, lab712, lab713, lab714, lab715, lab716, lab717, lab718, lab719, lab720, lab721, lab722, lab723, lab724, lab725 and/or lab726 as shown in FIG. 3.

In a tenth aspect, the invention is directed to a modular enzymatic system encoded by a nucleic acid sequence as described above. The modular enzymatic system preferably has functional activity in the biosynthesis of pederin-like and onnamide-like compounds and/or a polyketide moiety and/or a nonribosomal peptide moiety.

In an eleventh aspect, the present invention is directed to a vector comprising a nucleic acid consisting essentially of the Lab biosynthetic gene cluster derived from Labrenzia sp. and in particular from strain PHM005 or a vector comprising a nucleic acid sequence as described above.

In a twelfth aspect the present invention is directed to a recombinant host cell or a transgenic organism comprising said nucleic acid or containing said vector.

In a thirteenth aspect the present invention is directed to a method for producing pederin-like or onnamide-like compounds using a mutant of PHM005 or a recombinant host cell or a transgenic organism as described above, comprising the step of:
Culturing the mutant of PHM005 or the recombinant host cell or the transgenic organism under conditions to express the Lab biosynthetic gene cluster; and
Isolating the produced pederin-like and/or onnamide-like compounds.

Other aspects of the present invention are directed to the use of a nucleic acid as defined above in the preparation of a modified Lab biosynthetic gene cluster, to the use of a nucleic acid as defined above in the preparation of a pederin-like or onnamide-like compound and to processes for improving production of pederin-like and ormamide-like compounds in bacteria comprising the steps of a) culturing strain PHM005 in the presence of a mutagenic agent for a period of time sufficient to allow mutagenesis. and b) selecting said mutants by a change of the phenotype that results in an increased production of pederin-like or ormainide-like compounds. The mutagenic agent may be a chemical agent, such as daunorubicin and nitrosoguanidine; a physical agent, such as gamma radiation or ultraviolet radiation; or a biological agent, such as a transposon, for example. Exemplary modifications include knock-out of tailoring genes to avoid methylations and hidroxylations.

### BRIEF DESCRIPTION OF THE FIGURES AND THE SEQUENCES

FIG. 1. Electron microscopy (negative staining) of Labrenzia sp. PHM005. Cells in the mid-exponential growth phase were adsorbed on 400-mesh carbon-collodion-coated grids for 2 min, negatively stained with 2% uranyl acetate, imaged with a Jeol JEM 1011 transmission electron microscope operated at 100 kV and photographed with a CCD Gatan Erlangshen ES1000W camera.

FIG. 2. Neighbour joining tree based on 16S rRNA gene sequences that show the relationship between PHM005 and the type strains of closely related species of the genera Labrenzia and Stappia. The phylogenetic tree was generated by the Pairwise alignment based similarity coefficient and UPGMA for Cluster analysis using BioNumerics V7.5 (Applied Maths). The phylogenetic neighbors were identified and pairwise 16S rDNA gene sequence similarities calculated by comparison with the SILVA LTPs123 database.

FIG. 3. Map of Lab biosynthetic gene cluster. Total Lab gene cluster island: 69 Kb.

FIG. 4. ¹H NMR spectrum of compound 1 in CDCl₃.

FIG. 5. ¹³CNMR spectrum of compound 1 in CDCl₃.

FIG. 6. gCOSY spectrum of compound 1 in CDCl₃.

FIG. 7. TOCSY spectrum of compound 1 in CDCl₃.

FIG. 8. gHSQC spectrum of compound 1 in CDCl₃.

FIG. 9. LR-HSQMBC spectrum of compound 1 in CDCl₃.

FIG. 10. ROESY spectrum of compound 1 in CDCl₃.

The sequences mentioned in this application are listed in the attached sequence listing. These sequences are shortly summarized in the following:
SEQ ID NO: 1 Sequence (1355 bp) of 16S rRNA gene of Labrenzia sp. PHM005
SEQ ID NO: 2 nucleic acid sequence of the Lab biosynthetic gene cluster.
SEQ ID NO: 3 protein sequence of Lab706 putative acyl carrier protein.
SEQ ID NO: 4 protein sequence of Lab707 putative HMGS.
SEQ ID NO: 5 protein sequence of Lab708 PKS.
SEQ ID NO: 6 protein sequence of Lab709 TransAT PKS.
SEQ ID NO: 7 protein sequence of Lab710 putative acyl carrier protein.
SEQ ID NO: 8 protein sequence of Lab711 putative FAD oxigenase.
SEQ ID NO: 9 protein sequence of Lab712 putative oMethyltransferase.
SEQ ID NO: 10 protein sequence of Lab713 putative cytochrome P450.
SEQ ID NO: 11 protein sequence of Lab714 putative Malonyl CoA-ACP transacylase or FMT oxidoreductase.
SEQ ID NO: 12 protein sequence of Lab715 putative Malonyl CoA-ACP transacylase or acyltransferase.
SEQ ID NO: 13 protein sequence of Lab716 Malonyl CoA-ACP transacylase.
SEQ ID NO: 14 protein sequence of Lab717 Enoyl-CoA Hydratase.
SEQ ID NO: 15 protein sequence of Lab718 Beta-ketoacyl synthetase.
SEQ ID NO: 16 protein sequence of Lab719 TransAT PKS/NRPS.
SEQ ID NO: 17 protein sequence of Lab720 putative FAD monooxigenase.
SEQ ID NO: 18 protein sequence of Lab721, part of TransAT PKS.
SEQ ID NO: 19 protein sequence of Lab722, part of TransAT PKS.
SEQ ID NO: 20 protein sequence of Lab723, part of PKS.
SEQ ID NO: 21 protein sequence of Lab724, part of TransAT PKS/NRPS.
SEQ ID NO: 22 protein sequence of Lab725, part of PKS.
SEQ ID NO: 23 protein sequence of Lab726 putative oMethyltransferase.

### DETAILED DESCRIPTION OF PREFERRED EMBODIMENTS

The present invention relates to compounds of general formula I as defined above.

In the compounds defined by Markush formulae in this specification, the groups can be selected in accordance with the following guidance:

Alkyl groups may be branched or unbranched, and preferably have from 1 to about 12 carbon atoms. One more preferred class of alkyl groups has from 1 to about 6 carbon atoms. Even more preferred are alkyl groups having 1, 2, 3 or 4 carbon atoms. Methyl, ethyl, n-propyl, isopropyl, and butyl, including n-butyl, tert-butyl, sec-butyl and isobutyl are particularly preferred alkyl groups in the compounds of the present invention. As used herein, the term alkyl, unless otherwise stated, refers to both cyclic and non-cyclic groups, although cyclic groups will comprise at least three carbon ring members.

Alkenyl and alkynyl groups in the compounds of the present invention may be branched or unbranched, have one or more unsaturated linkages and from 2 to about 12 carbon atoms. One more preferred class of alkenyl and alkynyl groups has from 2 to about 6 carbon atoms. Even more preferred are alkenyl and alkynyl groups having 2, 3 or 4 carbon atoms. The terms alkenyl and alkynyl as used herein refer to both cyclic and noncyclic groups, although cyclic groups will comprise at least three carbon ring atoms.

Suitable aryl groups in the compounds of the present invention include single and multiple ring compounds, including multiple ring compounds that contain separate and/or fused aryl groups. Typical aryl groups contain from 1 to 3 separated or fused rings and from 6 to about 18 carbon ring atoms. Preferably aryl groups contain from 6 to about 14 carbon ring atoms. Specially preferred aryl groups include substituted or unsubstituted phenyl, substituted or unsubstituted naphthyl, substituted or unsubstituted biphenyl, substituted or unsubstituted phenanthryl and substituted or unsubstituted anthryl. The most preferred aryl group is substituted or unsubstituted phenyl.

Suitable heterocyclic groups include heteroaromatic and heteroalicyclic groups containing from 1 to 3 separated and/or fused rings and from 5 to about 18 ring atoms. Preferably heteroaromatic and heteroalicyclic groups contain from 5 to about 10 ring atoms, more preferably 5, 6 or 7 ring atoms. Suitable heteroaromatic groups in the compounds of the present invention contain one, two or three heteroatoms selected from N, O or S atoms and include, e.g., coumarinyl including 8-coumarinyl, quinolyl, including 8-quinolyl, isoquinolyl, pyridyl, pyrazinyl, pyrazolyl, pyrimidinyl, furyl, pyrrolyl, thienyl, thiazolyl, isothiazolyl, triazolyl, tetrazolyl, isoxazolyl, oxazolyl, imidazolyl, indolyl, isoindolyl, indazolyl, indolizinyl, phthalazinyl, pteridinyl, purinyl, oxadiazolyl, thiadiazolyl, furazanyl, pyridazinyl, triazinyl, cinnolinyl, benzimidazolyl, benzofuranyl, benzofurazanyl, benzothiophenyl, benzothiazolyl, benzoxazolyl, quinazolinyl, quinoxalinyl, naphthyridinyl, and furopyridyl. Suitable heteroalicyclic groups in the compounds of the present invention contain one, two or three heteroatoms selected form N, O or S atoms and include, e.g., pyrrolidinyl, tetrahydrofuranyl, tetrahydrothienyl, tetrahydrothiopyranyl, piperidyl, morpholinyl, thiomorpholinyl, thioxanyl, piperazinyl, azetidinyl, oxetanyl, thietanyl, homopiperidyl, oxepanyl, thiepanyl, oxazepinyl, diazepinyl, thiazepinyl, 1,2,3,6-tetrahydropyridil, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo [3.1.0] hexyl, 3-azabicyclo [4.1.0] heptyl, 3H-indolyl, and quinolizinyl.

The groups above mentioned may be substituted at one or more available positions by one or more suitable groups such as OR′, ═O, SR′, SOR′, SO₂R′, OSO₂R′, NO₂, NHR′, NR′R′, ═N—R′, N(R′)COR′, N(COR′)₂, N(R′)SO₂R, N(R′)C(═NR′)N(R′)R′, CN, halogen, COR′ COOR′, OCOR′, OCOOR′, OCONHR′, OCON(R′)R′, CON(R′)R′, CON(R′)OR′, CON(R′)SO₂R′, PO(OR′)₂, PO(OR′)R′, PO(OR′)(N(R′)R′), protected OH, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, and substituted or unsubstituted heterocyclic group, wherein each of the R′ groups is independently selected from the group consisting of hydrogen, OH, NO₂, NH₂, SH, CN, halogen, COH, COalkyl, COOH, substituted or unsubstituted C₁-C₁₂ alkyl, substituted or unsubstituted C₂-C₁₂ alkenyl, substituted or unsubstituted C₂-C₁₂ alkynyl, substituted or unsubstituted aryl, and substituted or unsubstituted heterocyclic group. Where such groups are themselves substituted, the substituents may be chose from the foregoing list.

Suitable halogen groups or substituents in the compounds of the present invention include F, Cl, Br, and I.

Suitable protecting groups for OH, including protecting groups for 1,2-diols, are well known for the person skilled in the art. A general review of protecting groups in organic chemistry is provided by Wuts, P G M and Greene T W in Protecting Groups in Organic Synthesis 4^{th} Ed. Wiley-Interscience, and by Kocienski P J in Protecting Groups, 3^{th} Ed. Georg Thieme Verlag. These references provide sections on protecting groups for OH. All these references are incorporated by reference in their entirely.

Within the scope of the present invention an OH protecting group is defined to be the O-bonded moiety resulting from the protection of the OH group through the formation of a suitable protected OH group. Examples of such protected OH include ethers, silyl ethers, esters, sulfonates, sulfenates and sulfinates, carbonates and carbamates. In the case of ethers the protecting group for the OH can be selected from methyl, methoxymethyl, methylthiomethyl, (phenyldimethylsilyl)methoxymethyl, benzyloxymethyl, p-methoxybenzyloxymethyl, [(3,4-dimethoxybenzypoxy]methyl, p-nitrobenzyloxymethyl, o-nitrobenzyloxymethyl, [(R)-1-(2-nitrophenyl)ethoxy]methyl, (4-methoxyphenoxy)methyl, guaiacolmethyl, [(p-phenylphenyl)-oxy]methyl, t-butoxymethyl, 4-pentenyloxymethyl, siloxymethyl, 2-methoxyethoxymethyl, 2-cyanoethoxymethyl, bis(2-chloroethoxy)methyl, 2,2,2-trichoroethoxymethyl, 2-(trimethylsilyl)-ethoxymethyl, menthoxymethyl, O-bis(2-acetoxyethoxy)methyl, tetrahydropyranyl, fluorous tetrahydropyranyl, 3-bromotetrahydropyranyl, tetrahydrothiopyranyl, 1-methoxycyclohexyl, 4-methoxytetrahydropyranyl, 4-methoxytetrahydrothiopyranyl, 4-methoxytetrahydrothiopyranyl S,S-dioxide, 1-[(2-chloro-4-methyl)phenyl]-4-methoxypiperidin-4-yl, 1-(2-fluorophenyl)-4-methoxypiperidin-4-yl, 1-(4-chlorophenyl)-4-methoxypiperidin-4-yl, 1,4-dioxan-2-yl, tetrahydrofuranyl, tetrahydrothiofuranyl, 2,3,3α,4,5,6,7,7α-octahydro-7,8,8-trimethyl-4,7-methanobenzofuran-2-yl, 1-ethoxyethyl, 1-(2-chloroethoxy)ethyl, 2-hydroxyethyl, 2-bromoethyl, 1-[2-(trimethylsilypethoxy] ethyl, 1-methyl-1-methoxyethyl, 1-methyl-1-benzyloxy ethyl, 1-methyl-1-benzyloxy-2-fluoroethyl, 1-methyl-1-phenoxy ethyl, 2,2,2-trichloroethyl, 1,1-dianisyl-2,2,2-trichloroethyl, 1,1,1,3,3,3-hexafluoro-2-phenylisopropyl, 1-(2-cyanoethoxy)ethyl, 2-trimethylsilylethyl, 2-(benzylthio)ethyl, 2-(phenylselenyl)ethyl, t-butyl, cyclohexyl, 1-methyl-1′-cyclopropylmethyl, allyl, prenyl, cinnamyl, 2-phenallyl, propargyl, p-chlorophenyl, p-methoxyphenyl, p-nitrophenyl, 2,4-dinitrophenyl, 2,3,5,6-tetrafluoro-4-(trifluoromethyl)phenyl, benzyl, p-methoxybenzyl, 3,4-dimethoxybenzyl, 2,6-dimethoxybenzyl, o-nitrobenzyl, p-nitrobenzyl, pentadienylnitrobenzyl, pentadienylnitropiperonyl, halobenzyl, 2,6-dichlorobenzyl, 2,4-dichlorobenzyl, 2,6-difluorobenzyl, p-cyanobenzyl, fluorous benzyl, 4-fluorousalkoxybenzyl, trimethylsilylxylyl, p-phenylbenzyl, 2-phenyl-2-propyl, p-acylaminobenzyl, p-azidobenzyl, 4.azido-3-chlorobenzyl, 2-trifluoromethylbenzyl, 4-trifluoromethylbenzyl, p-(methylsulfinyl)benzyl, p-siletanylbenzyl, 4-acetoxybenzyl, 4-(2-trimethylsilyl)ethoxymethoxybenzyl, 2-naphthylmethyl, 2-picolyl, 4-picolyl, 3-methyl-2-picolyl N-oxide, 2-quinolinylmethyl, 6-methoxy-2-(4-methylphenyl-4-quinolinemethyl, 1-pyrenylmethyl, diphenylmethyl, 4-methoxydiphenylmethyl, 4-phenyldiphenylmethyl, p,p′-dinitrobenzhydryl, 5-dibenzosuberyl, triphenylmethyl, tris(4-t-butylphenyl)methyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxy)phenyldiphenylmethyl, 4,4′,4″-tris(4,5-dichlorophthalimidophenyl)methyl, 4,4′4″-tris(levulinoyloxyphenyl)methyl, 4,4′,4″-tris(benzoyloxyphenyl)methyl, 4,4′-dimethoxy -3″-[(imidazolylmethypitrityl, 4,4′-dimethoxy-3″-[N-(imidazolylethyl)carbamoyl]trityl, bis(4-methoxyphenyl)-1′-pyrenylmethyl, 4-(17-tetrabenzo [a,c,g,i]fluorenylmethyl)-4,4″-dimethoxytrityl, 9-anthryl, 9-(9-phenyl)xan-thenyl, 9-phenylthioxanthyl, 9-(9-phenyl-10-oxo)anthryl, 1,3-benzodithiolan-2-yl, and 4,5-bis(ethoxy carbonyl)-[1,3]-dioxolan-2-yl, benzisothiazolyl S,S-dioxide. In the case of silyl ethers the protecting group for the OH can be selected from trimethylsilyl, triethylsilyl, triisopropylsilyl, dimethylisopropylsilyl, diethylisopropylsilyl, dimethylhexylsilyl, 2-norbornyldimethylsilyl, t-butyldimethylsilyl, t-butyldiphenylsilyl, tribenzylsilyl, tri-p-xylylsilyl, triphenylsilyl, diphenylmethylsilyl, di-t-butylmethylsilyl, bis(t-butyl)-1-pyrenylmethoxy silyl, tris(trimethylsilyl) silyl, (2-hydroxystyryl)dimethylsilyl, (2-hydroxystyryl)diisopropylsilyl, t-bu-tylmethoxyphenylsilyl, t-butoxydiphenylsilyl, 1,1,3 ,3-tetraisopropyl-3-[2-(triphenylmethoxy)e-thoxy]disiloxane-1-yl, and fluorous silyl. In the case of esters the protecting group for the OH together with the oxygen atom of the unprotected OH to which it is attached form an ester that can be selected from formate, benzoylformate, acetate, chloroacetate, dichloroacetate, trichloroacetate, trichloroacetamidate, trifluoroacetate, methoxy acetate, triphenylmethoxy ace-tate, phenoxyacetate, p-chlorophenoxyacetate, phenylacetate, diphenylacetate, 3-phenylpropionate, bisfluorous chain type propanoyl, 4-pentenoate, 4-oxopentanoate, 4,4-(ethylenedithio)pentanoate, 5-[3-bis(4-methoxyphenyyl)hydroxymethylphenoxy]levulinate, pivaloate, 1-adamantoate, crotonate, 4-methoxycrotonate, benzoate, p-phenylbenzoate, 2,4,6-trimethylbenzoate, 4-bromobenzoate, 2,5-difluorobenzoate, p-nitrobenzoate, picolinate, nicotinate, 2-(azidomethyl)benzoate, 4-azidobutyrate, (2-azidomethyl)phenylacetate, 2-{[tritylthio)oxy]methyl}benzoate, 2-{[(4-methoxytritylthio)oxy]methyl}benzoate, 2-{[methyl (tritylthio)amino]methyl}benzoate, 2-{{[(4-methoxytrity)thio]methylamino]-methyl}benzoate, 2-(allyloxy)phenylacetate, 2-(prenyloxymethyl)benzoate, 6-(levulinyloxymethyl)-3-methoxy-2-nitrobenzoate, 6-(levulinyloxymethyl)-3-methoxy-4-nitrobenzoate, 4-benzyloxybutyrate, 4-trialkylsilyloxybutyrate, 4-acetoxy-2,2-dimethylbutyrate, 2,2-dimethyl-4-pentenoate, 2-iodobenzoate, 4-nitro-4-methylpentanoate, o-(dibromomethyl)benzoate, 2-formylbenzenesulfonate, 4-(methylthiomethoxy)butyrate, 2-(methylthiomethoxymethyl)benzoate, 2-(chloroacetoxymethyl)benzoate, 2-[(2-choroaceto xy)ethyl]benzoate, 2-[2-benzyloxy)ethyl]benzoate, 2-[2-(4-methoxybenzyloxy)ethyl]benzoate, 2,6-dichloro-4-methylphenoxy acetate, 2,6-dichloro-4-(1,1,3 ,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxycarbonyl)benzoate, α-naphthoate, nitrate, alkyl N,N,N′,N′-tetramethylphosphorodiamidate, and 2-chlorobenzoate. In the case of sulfonates, sulfenates and sulfinates the protecting group for the OH together with the oxygen atom of the unprotected OH to which it is attached form a sulfonate, sulfenate or sulfinate that can be selected from sulfate, allylsulfonate, methanesulfonate, benzylsulfonate, tosylate, 2-[(4-nitrophenyl)ethyl]sulfonate, 2-trifluoromethylbenzenesulfonate, 4-monomethoxytritylsulfenate, alkyl 2,4-dinitrophenylsul-fenate, 2,2,5,5-tetramethylpyrrolidin-3-one-1-sulfinate, and dimethylphosphinothiolyl. In the case of carbonates the protecting group for the OH together with the oxygen atom of the unprotected OH to which it is attached form a carbonate that can be selected from methyl carbonate, methoxymethyl carbonate, 9-fluorenylmethyl carbonate, ethyl carbonate, bromoethyl carbonate, 2-(methylthiomethoxy)ethyl carbonate, 2,2,2-trichloroethyl carbonate, 1,1-dimethyl-2,2,2-trichloroethyl carbonate, 2-(trimethylsilyl)ethyl carbonate, 2-[dimethyl(2-naph-thylmethyl)silyl]ethyl carbonate, 2-(phenylsulfonyl)ethyl carbonate, 2-(triphenylphos-phonio)ethyl carbonate, cis-[4-[[(methoxytrityl)sulfenyl]oxy]tetrahydrofuran-3-yl]oxy carbonate, isobutyl carbonate, t-butyl carbonate, vinyl carbonate, allyl carbonate, cinnamyl carbonate, propargyl carbonate, p-chlorophenyl carbonate, p-nitrophenyl carbonate, 4-ethoxy-1-naphthyl carbonate, 6-bromo-7-hydroxycoumarin-4-ylmethyl carbonate, benzyl carbonate, o-nitrobenzyl carbonate, p-nitrobenzyl carbonate, p-methoxybenzyl carbonate, 3,4-dimethoxybenzyl carbonate, anthraquinon-2-ylmethyl carbonate, 2-dansylethyl carbonate, 2-(4-nitrophenyl)ethyl carbonate, 2-(2,4-dinitrophenyl)ethyl carbonate, 2-(2-nitrophenyl)propyl carbonate, alkyl 2-(3,4-methylenedioxy-6-nitrophenyl)propyl carbonate, 2-cyano-1-phenylethyl carbonate, 2-(2-pyridylamino-1-phenylethyl carbonate, 2-[N-methyl-N-(2-pyridyl)]amino-1-phenylethyl carbonate, phenacyl carbonate, 3′,5′-dimethoxybenzoin carbonate, methyl dithiocarbonate, and S-benzyl thiocarbonate. And in the case of carbamates the protecting group for the OH together with the oxygen atom of the unprotected OH to which it is attached forms a carbamate that can be selected from dimethylthiocarbamate, N-phenylcarbamate, and N-methyl-N-(o-nitrophenyl)carbamate.

Within the scope of the present invention an 1,2-diol protecting group is defined to be the O-bonded moiety resulting from the simultaneous protection of the 1,2-diol through the formation of a protected 1,2-diol. Examples of such protected 1,2-diols include cyclic acetals and ketals, cyclic ortho esters, silyl derivatives, dialkylsilylene derivatives, cyclic carbonates, cyclic boronates. Examples of cyclic acetals and ketals include methylene acetal, ethylidene acetal, t-butylmethylidene acetal, 1-t-buylethylidene ketal, 1-phenylethylidene ketal, 2-(methoxycarbonyl)ethylidene (Mocdene) acetal, or 2-(t-butylcarbonyl)ethylidene (Bocdene) acetal, phenylsulfonylethylidene acetal, 2,2,2-trichloroethylidene acetal, 3-(benzyloxy)propyl acetal, acrolein acetal, acetonide (isopropylidene ketal), cyclopentylidene ketal, cyclohexylidene ketal, cycloheptylidene ketal, benzylidene acetal, p-methoxybenzylidene acetal, 1-(4-methoxyphenyl)ethylidene ketal, 2,4-dimethoxybenzylidene acetal, 3,4-dimethoxybenzylidene acetal, p-acetoxybenzylidene acetal, 4-(t-butyldimethylsilyloxy)benzylidene acetal, 2-nitrobenzylide acetal, 4-nitrobenzylidene acetal, mesitylene acetal, 6-bromo-7-hydroxycoumarin-2-ylmethylidene acetal, 1-naphthaladehyde acetal, 2-naphthaldehyde acetal, 9-anthracene acetal, benzophenone ketal, di-(p-anisyl)methylidene acetal, xanthen-9-ylidene ketal, 2,7-dimethylxanthen-9-ylidene ketal, diphenylmethylene ketal, camphor ketal, and menthone ketal. Examples of cyclic ortho esters include methoxymethylene acetal, ethoxymethylene acetal, 2-oxacyclopentylidene ortho ester, dimethoxymethylene ortho ester, 1-methoxyethylidene ortho ester, 1-ethoxyethylidene ortho ester, phthalide ortho ester, 1,2-dimethoxyethylidene ortho ester, cc-methoxybenzylidene ortho ester, 1-(N,N-dimethylamino)ethylidene derivative, α-(N,N-dimethylamino)benzylidene derivative, butane 2-3-bisacetal (BBA), cyclohexane-1,2-diacetal (CDA), and dispiroketals. Examples of silyl derivatives include di-t-butylsilylene group (DTBS(OR)₂), 1-(cyclohexyl)-1-(methypsilylene (Cy)(Me)Si(OR)₂, di-isopropylsilylene (i-propyl) 2Si(OR)₂, dicyclohexylsilylene (Cy)₂Si(OR)₂,1,3-(1,1,3,3-tetraisopropyldisiloxanylidene) derivative (TIPDS(OR)₂), 1,1,3,3-tetra-t-butoxydisiloxanylidene derivative (TBDS(OR)₂), methylene-bis-(diisopropylsilanoxanylidene) (MDPS(OR)₂), and 1,1,4,4-tetraphenyl-1,4-disilanylidene (SIBA(OR)₂). Examples of cyclic boronates include methyl boronate, ethyl boronate, phenyl boronate, and o-acetamidophenyl boranate.

The mention of these groups should not be interpreted as a limitation of the scope of the invention, since they have been mentioned as a mere illustration of protecting groups for OH, but further groups having said function may be known by the skilled person in the art, and they are to be understood to be also encompassed by the present invention.

The term “pharmaceutically acceptable salt” refers to any pharmaceutically acceptable salt which, upon administration to the patient is capable of providing (directly or indirectly) a compound as described herein. However, it will be appreciated that non-pharmaceutically acceptable salts also fall within the scope of the invention since those may be useful in the preparation of pharmaceutically acceptable salts. The preparation of salts can be carried out by methods known in the art.

For instance, pharmaceutically acceptable salts of compounds provided herein are synthesized from the parent compound, which contains a basic or acidic moiety, by conventional chemical methods. Generally such salts are, for example, prepared by reacting the free acid or base forms of these compounds with a stoichiometric amount of the appropriate base or acid in water or in an organic solvent or in a mixture of both. Generally, nonaqueous media like ether, ethyl acetate, ethanol, 2-propanol or acetonitrile are preferred. Examples of the acid addition salts include mineral acid addition salts such as, for example, hydrochloride, hydrobromide, hydroiodide, sulfate, nitrate, phosphate, and organic acid addition salts such as, for example, acetate, trifluoroacetate, maleate, fumarate, citrate, oxalate, succinate, tartrate, malate, mandelate, methanesulfonate and p-toluenesulfonate. Examples of the alkali addition salts include inorganic salts such as, for example, sodium, potassium, calcium and ammonium salts, and organic alkali salts such as, for example, ethylenediamine, ethanolamine, N,N-dialkylenethanolamine, triethanolamine and basic aminoacids salts.

The compounds of the invention may be in crystalline or amorphous form either as free compounds or as solvates (e.g. hydrates, alcoholates, particularly methanolates) and it is intended that any of these forms are within the scope of the present invention. Methods of solvation are generally known within the art. The compounds of the invention may present different polymorphic forms, and it is intended that the invention encompasses all such forms.

Any compound referred to herein is intended to represent such specific compound as well as certain variations or forms. In particular, compounds referred to herein may have asymmetric centers and therefore exist in different enantiomeric or diastereomeric forms. Thus, any given compound referred to herein is intended to represent any one of a racemate, one or more enantiomeric forms, one or more diastereomeric forms, and mixtures thereof. Likewise, stereoisomerism or geometric isomerism about the double bond is also possible, therefore in some cases the molecule could exist as (E)-isomer or (Z)-isomer (trans and cis isomers). If the molecule contains several double bonds, each double bond will have its own stereoisomerism, that could be the same, or different to, the stereoisomerism of the other double bonds of the molecule. Furthermore, compounds referred to herein may exist as atropisomers. All the stereoisomers including enantiomers, diastereoisomers, geometric isomers and atropisomers of the compounds referred to herein, and mixtures thereof, are considered within the scope of the present invention.

Furthermore, any compound referred to herein may exist as tautomers. Specifically, the term tautomers refer to one of two or more structural isomers of a compound that exist in equilibrium and are readily converted from one isomeric form to another. Common tautomeric pairs are amine-imine, amide-imidic acid, keto-enol, lactam-lactim, etc.

Unless otherwise stated, the compounds of the invention are also meant to include isotopically-labelled forms i.e. compounds which differ only in the presence of one or more isotopically-enriched atoms. For example, compounds having the present structures except for the replacement of at least one hydrogen atom by a deuterium or tritium, or the replacement of at least one carbon atom by ¹³C- or ¹⁴C-enriched carbon, or the replacement of at least one nitrogen atom by ¹⁵N-enriched nitrogen are within the scope of this invention.

To provide a more concise description, some of the quantitative expressions given herein are not qualified with the term “about”. It is understood that, whether the term “about” is used explicitly or nor, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value.

More particularly, preferred compounds of formula I are those also having general formula III or a pharmaceutically acceptable salt, tautomer, and stereoisomer thereof.

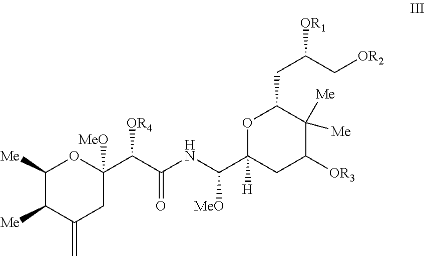

wherein R₁, R₂, R₃ and R₄ are as defined above in general formula I.

In compounds of general formula I and III, particularly preferred R₁ is selected from hydrogen and substituted or unsubstituted C₁-C₁₂ alkyl. More preferably R₁ is selected from hydrogen and substituted or unsubstituted C₁-C₆ alkyl. Even more preferably, R₁ is selected from hydrogen, methyl, ethyl, n-propyl, isopropyl, n-butyl, tert-butyl, sec-butyl, and isobutyl. Most preferred R₁ are hydrogen and methyl.

In compounds of general formula I and III, particularly preferred R₂ is selected from hydrogen and —C(═O)Rₐ, wherein Rₐ is substituted or unsubstituted C₁-C₁₂ alkyl. More preferred Rₐ is substituted or unsubstituted C₁-C₆ alkyl. Even more preferably Rₐ is selected from methyl, ethyl, n-propyl, isopropyl, n-butyl, tert-butyl, sec-butyl and isobutyl. Most preferred R₂ are hydrogen and acetyl.

In compounds of general formula I and III, particularly preferred R₃ and R₄ are independently selected from hydrogen and —C(═O)Rₐ, wherein Rₐ at each occurrence is independently selected from substituted or unsubstituted C₁-C₁₂ alkyl. More preferably Rₐ at each occurrence is independently selected from substituted or unsubstituted C₁-C₆ alkyl. Even more preferably, Rₐ at each occurrence is independently selected from methyl, ethyl, n-propyl, isopropyl, n-butyl, tert-butyl, sec-butyl and isobutyl. Most preferably R₃ and R₄ are independently selected from hydrogen and acetyl.

In additional preferred embodiments, the preferences described above for the different substituents are combined. The present invention is also directed to such combinations of preferred substitutions in the general formula I and III above.

In one embodiment, R₁ is selected from substituted or unsubstituted C₁-C₆ alkyl and R₂ is hydrogen.

In another embodiment, R₁ is selected from substituted or unsubstituted C₁-C₆ alkyl and R₂ is —C(═O)Rₐ, wherein Rₐ is substituted or unsubstituted C₁-C₁₂ alkyl.

In a further embodiment, both R₁ and R₂ are hydrogen.

In the present description and definitions, when there are several groups Rₐ, R_{b}, R_{c}, R_{d} or R′ present in the compounds of the invention, and unless it is stated explicitly so, it should be understood that they can be each independently different within the given definition, i.e. Rₐ does not represent necessarily the same group simultaneously in a given compound of the invention.

Particularly preferred compounds of the invention are the following

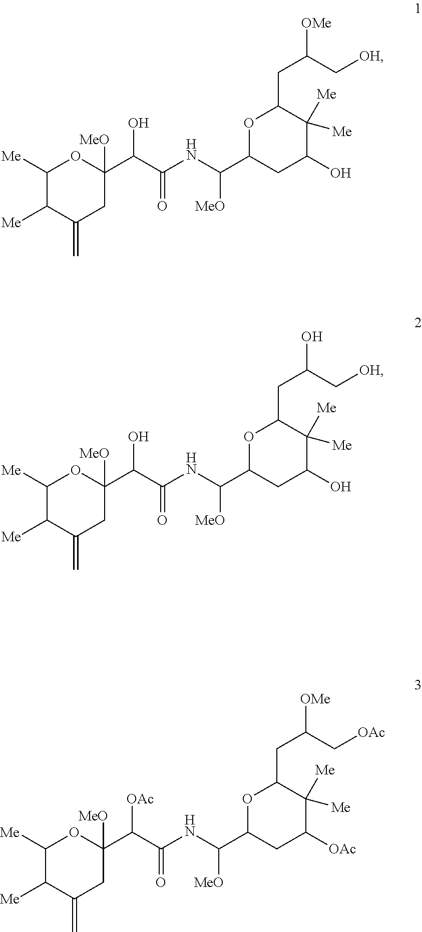 [SMILES: C=C1CC(OC)(C(O)C(=O)NC(OC)C2CC(O)C(C)(C)C(CC(CO)OC)O2)OC(C)C1C ; C=C1CC(OC)(C(O)C(=O)NC(OC)C2CC(O)C(C)(C)C(CC(O)CO)O2)OC(C)C1C ; C=C1CC(OC)(C(OC(C)=O)C(=O)NC(OC)C2CC(OC(C)=O)C(C)(C)C(CC(COC(C)=O)OC)O2)OC(C)C1C]

or pharmaceutically acceptable salts, tautomers or stereoisomers thereof.

Most preferred compounds of the invention are the following:

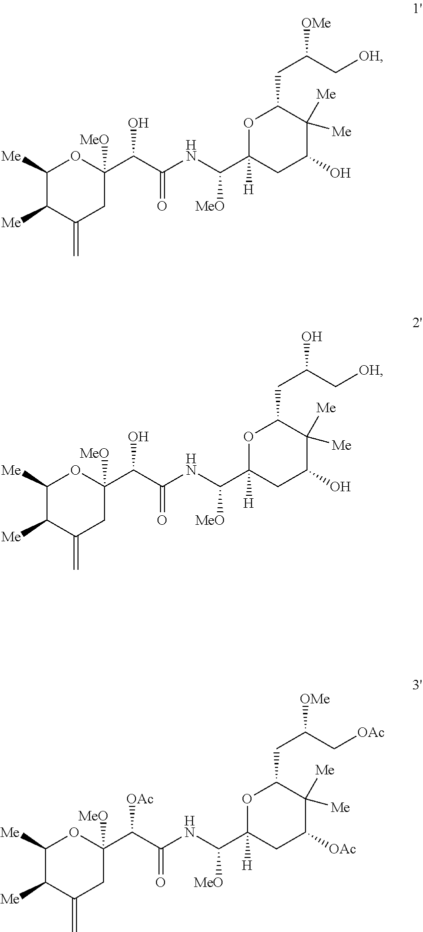

or pharmaceutically acceptable salts, tautomers or stereoisomers thereof.

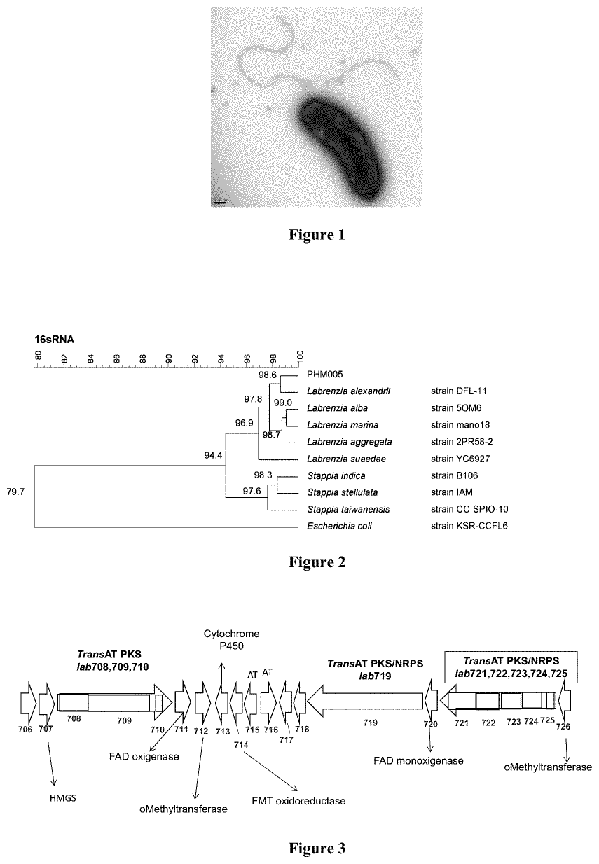

Compounds 1 and 2 were isolated from Labrenzia sp., named strain PHM005. This alphaproteobacteria was isolated from a marine sediment sample collected in the Indian Ocean. Observation of the cells by transmission electron microscopy (FIG. 1) allowed the identification of motile rods (0.6-0.8 μm wide and 1.6-2.1 μm long) with single, subpolar inserted flagella. A culture of this strain has been deposited in the CECT (“Colección Española de Cultivos Tipo”) at the University of Valencia, Spain, under the accession number CECT-9225. This deposit has been made under the provisions of the Budapest Treaty.

The bacteria is clearly marine salt dependent since it needs more than 2.5% NaCl to grow, with the optimal concentration of marine salt for production of 1 being 36 g/L, similar to ocean conditions. Colonies on Marine Agar 2216 (DIFCO) are beige, almost transparent, smooth and with entire margin. After three weeks the colonies become darker-brown, maybe due to the effect of bacteriochlorophyll a and carotenoid production, as described for Labrenzia alexandrii DFL-11^{T} (Biebl and co-workers, Evol, Microbiol, 2007, 57, 1095-1107).

For the isolation of the producer microorganism, all the manipulations were carried out in aseptic conditions. PHM005 was isolated from a sediment frozen sample spread directly on Petri dishes with a sea salt medium of the following composition (g/L): marine salts (Tropic Marin® PRO-REEF, 27; agar, 16; supplemented with cycloheximide 0.2 mg/mL. The plates were incubated at 28° C. for three weeks under atmospheric pressure. After this period a slightly brown colony was picket and transferred to the same sea salt medium to confirm the purity and start taxonomy and fermentation studies.

A taxonomic evaluation of PHM005 was conducted by partial sequence of 16S rRNA following standard procedures. PHM005 was grown in marine broth (DIFCO 1196) for 72 hours. Cells were recovered and lysed by boiling with 4% NP40 for 10 minutes. Cell debris was discarded by centrifugation. The 16S rRNA was amplified by the polymerase chain reaction using the bacterial primers F1 and R₅ described by Cook and Myers (International Journal of Systematics and Evolutionary Microbiology, 2003, 53, 1907-1915. The nearly full-length 16S rRNA gene sequence obtained is shown in SEQ NO: 1.

The phylogenetic tree was generated by the Pairwise alignment based similarity coefficient and UPGMA for Cluster analysis using BioNumerics V7.5 for Cluster Analysis. The phylogenetic neighbors were identified and pairwise 16S rRNA gene sequence similarities calculated by comparison with the SILVA LTPs123 database. The phylogenetic tree is shown in FIG. 2.

PHM005 produces compounds 1 and 2 when it is cultured under controlled conditions in a suitable medium. This strain clearly needs marine salt to grow. This strain is preferably grown in a conventional aqueous nutrient medium. The culture must be driven in aerobic conditions and the production of compounds 1 and 2 should start after 3 days of growth controlling temperature between 26-28° C. Conventional fermentation tanks have been found to be well suited for carrying out the cultivation of this organism. The addition of nutrients and pH control as well as antifoaming agents during the different stages of fermentation may be needed for increasing production and avoid foaming.

Compounds of the present invention can be produced starting from a colony or a frozen pure culture of strain PHM005 for developing enough biomass. This step may be repeated several times as needed and the material collected will be used as an inoculum to seed one or several fermentation flasks or tanks with the appropriate culture medium. These flasks or tanks can be used for developing the inoculum or for the production stage, depending on the broth volume needed. Sometimes the production medium may be different from the ones used for inoculum development.

Compounds of the present invention can be isolated from the fermentation broth mainly from cells and from the supernatant of strain PHM005 by extraction with a suitable mixture of solvents or absorbing in adequate resins.

Separation and purification of the present invention from the crude active extract can be performed using the proper combination of conventional chromatographic techniques.

Additionally, compounds of the invention can be obtained by modifying those already obtained from the natural source or by further modifying those already modified by using a variety of chemical reactions. Thus, hydroxyl groups can be acylated by standard coupling or acylation procedures, for instance by using acetyl chloride or acetic anhydride in pyridine or the like. Formate groups can be obtained by reacting the corresponding alkoxyde with acetic formic anhydride. Carbamates can be obtained by heating hydroxyl precursors with isocyanates. Carbonates can be obtained by using the corresponding anhydride and an activator such as Mg(CLO₄)₂ or Zn(OAc)₂, Hydroxyl groups can also be converted into alkoxy groups by alkylation using an alkyl bromide iodide or sulfonate or into amino lower alkoxy groups by using, for instance, a protected 2-bromoethylamine. When necessary, appropriate protecting groups can be used on the substituents to ensure that reactive groups are not affected and to all selective functionalization of the hydroxyl groups. The procedures and reagents needed to prepare these derivatives are known to the skilled person and can be found in general textbooks such as March's Advanced Organic Chemistry 7^{th} Edition 2013, Wiley Interscience.

An important feature of the above described compounds of formula I and III is their bioactivity and in particular their cytotoxic activity against tumor cells. Thus, with this invention we provide pharmaceutical compositions of compounds of general formula I and III, or a pharmaceutically acceptable salt, tautomer or stereoisomer thereof that possess cytotoxicity activities and their use as anticancer agents. The present invention further provides pharmaceutical compositions comprising a compound of general formula I and III, or a pharmaceutically acceptable salt, tautomer or stereoisomer thereof, with a pharmaceutically acceptable carrier or diluent.

Examples of pharmaceutical compositions include any solid (tablet, pills, capsules, granules, powder for vials, etc.) or liquid (solutions, suspensions or emulsions) composition for oral, topical or parenteral administration.

Administration of the compounds or compositions of the present invention may be by any suitable method, such as intravenous infusion, oral preparations, and intraperitoneal and intravenous administration. We prefer that infusion times of up to 24 hours are used, more preferably 1-12 hours, with 1-6 hours most preferred. Short infusion times which allow treatment to be carried out without an overnight stay in hospital are especially desirable. However, infusion may be 12 to 24 hours or even longer if required. Infusion may be carried out at suitable intervals of say 1 to 4 weeks. Pharmaceutical compositions containing compounds of the invention may be delivered by liposome or nanosphere encapsulation, in sustained release formulations or by other standard delivery means.

The correct dosage of the compounds will vary according to the particular formulation, the mode of application, ant the particular status, host and tumor being treated. Other factors like age, body weight, sex, diet, time of administration, rate of excretion, condition of the host, drug combinations, reaction sensitivities and severity of the disease shall be taken into account. Administration can be carried out continuously or periodically within the maximum tolerated dose.

As used herein, the terms “treat”, “treating” and “treatment” include the eradication, removal, modification, or control of a tumor or primary, regional, or metastatic cancer cells or tissue and the minimization of delay of the spread of cancer.

The compounds of the invention have anticancer activity against several cancer types which include, but are not limited to, lung cancer, colon cancer, breast cancer and pancreas cancer.

Thus in alternative embodiments of the present invention, the pharmaceutical composition comprising a compound of formula I and III as defined above is for the treatment of lung cancer, colon cancer, breast cancer or pancreas cancer.

In a sixth aspect, the present invention is directed to a process for the production of compounds of formula II. Preferred processes according to this aspect of the invention are those that produce a compound also having formula IV

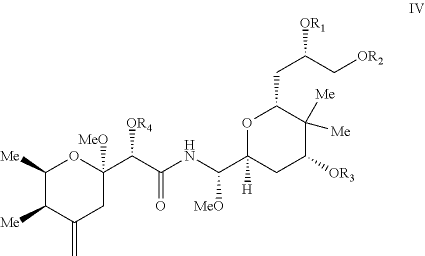

or a pharmaceutically acceptable salt, tautomer or stereoisomer thereof;

wherein R₁, R₂, R₃ and R₄ are as defined above in general formula II.

In processes for the synthesis of compounds of formula II and IV, particularly preferred R₁ is selected from hydrogen, substituted or unsubstituted C₁-C₁₂ alkyl, and —C(═O)Rₐ where Rₐ is substituted or unsubstituted C₁-C₁₂ alkyl. More preferably R₁ is selected from hydrogen, substituted or unsubstituted C₁-C₆ alkyl and —C(═O)Rₐ where Rₐ is substituted or unsubstituted C₁-C₆ alkyl. Even more preferably, R₁ is selected from hydrogen, methyl, ethyl, n-propyl, isopropyl, n-butyl, tert-butyl, sec-butyl, isobutyl and —C(═O)Rₐ wherein Rₐ is selected from methyl, ethyl, n-propyl, isopropyl, n-butyl, tert-butyl, sec-butyl and isobutyl. Most preferred R₁ is selected from hydrogen and methyl.

In processes for the synthesis of compounds of formula II and IV, particularly preferred R₂ is selected from hydrogen, substituted or unsubstituted C₁-C₁₂ alkyl, and —C(═O)Rₐ where Rₐ is substituted or unsubstituted C₁-C₁₂ alkyl. More preferably R₂ is selected from hydrogen, substituted or unsubstituted C₁-C₆ alkyl and —(C═O)Rₐ where Rₐ is substituted or unsubstituted C₁-C₆ alkyl. Even more preferably, R₂ is selected from hydrogen, methyl, ethyl, n-propyl, isopropyl, n-butyl, tert-butyl, sec-butyl, isobutyl, and —C(═O)Rₐ where Rₐ is selected from methyl, ethyl, n-propyl, isopropyl, n-butyl, tert-butyl, sec-butyl and isobutyl. Most preferred R₂ are hydrogen, methyl and acetyl.

In processes for the synthesis of compounds of formula II and IV, particularly preferred R₃ and R₄ are independently selected from hydrogen and —C(═O)Rₐ, wherein Rₐ at each occurrence is independently selected from substituted or unsubstituted C₁-C₁₂ alkyl. More preferably Rₐ at each occurrence is independently selected form substituted or unsubstituted C₁-C₆ alkyl. Even more preferably, Rₐ is selected from methyl, ethyl, n-propyl, isopropyl, n-butyl, tert-butyl, sec-butyl and isobutyl. Most preferred R₃ and R₄ are independently selected from hydrogen and acetyl.

In processes for the synthesis of compounds of formula II and IV, particularly preferred compounds 1 and 2 have, respectively, the following relative stereochemistry:

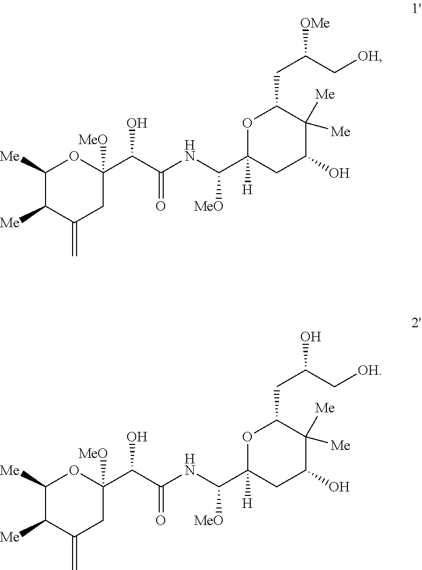 [SMILES: C=C1C[C@](OC)([C@H](O)C(=O)N[C@@H](OC)[C@@H]2C[C@@H](O)C(C)(C)[C@@H](C[C@@H](CO)OC)O2)O[C@H](C)[C@@H]1C ; C=C1C[C@](OC)([C@H](O)C(=O)N[C@@H](OC)[C@@H]2C[C@@H](O)C(C)(C)[C@@H](C[C@H](O)CO)O2)O[C@H](C)[C@@H]1C]

In additional preferred embodiments, the preferences described above for the different substituents are combined. The present invention is also directed to such combinations of preferred substitutions in the processes for the synthesis of compounds of formula II and IV above.

In a more preferred embodiment of this aspect of the invention the compound of formula II or IV is pederin.

In an even more preferred embodiment, pederin is obtained from compound 1′ by:
Protecting all the hydroxy groups in compound 1′ with a protecting group for —OH suitable to be selectively removed from a protected primary OH in presence of protected secondary OH. Examples of such protecting group include trimethylsilyl, triethylsilyl, triisopropylsilyl, and tert-butyldimethylsilyl. Most preferred protecting group for this step is tert-butyldimethylsilyl;
Selectively removing the primary OH protecting group;
Methylating the resulting primary hydroxy group with a suitable methylation reagent; and
Removing the other protecting groups for OH.

In another more preferred embodiment, pederin is obtained from compound 2′ by:
Protecting the 1,2-diol group with a suitable protecting group for 1,2-diols. Examples of suitable protecting groups for 1,2-diols include, but are not limited to, those groups that after reaction with the corresponding 1,2-diol generate Mocdene acetal, Bocdene acetal, acrolein acetal, benzylidene acetal, (t-butyldimethylsilyloxy)benzylidene acetal, mesitylene acetal, methoxymethylene acetal, ethoxymethylene acetal, cyclic carbonates, methyl boronate and ethyl boronate. More preferred protecting groups for this step are those that generate a Mocdene acetal, Bocdene acetal, benzylidene acetal, and cyclic carbonates being the protecting group that generates a benzylidene acetal the most preferred;
Protecting the other hydroxy groups with a protecting group for —OH that is orthogonal with the 1,2-diol protecting group of previous step. Examples of protecting groups for OH suitable for this step are trimethylsilyl, triethylsilyl, triisopropylsilyl tert-butyldimethylsilyl, and acetyl. Most preferred protecting group for this step are tert-butyldimethylsilyl and acetyl;
Removing the 1,2-diol protecting group;
Methylating the resulting 1,2-diol with a suitable methylation reagent; and
Removing the other protecting groups for OH.

Examples of suitable methylation reagents include methyl iodide, methyl bromide, dimethylsulfate, and methyl triflate.

The isolated nucleic acid according to the eighth aspect of the invention is preferably derived from Labrenzia sp, and in particular from strain PHM005.

The complete genome sequence of this bacterium revealed the biosynthetic gene cluster responsible for the pederin and onnamide synthesis. Bioinformatic analysis was used to predict the function of the genes in the cluster.

This gene cluster, named Lab gene cluster, is a Trans-AT hybrid polyketide synthase/non ribosomal synthetase (PKS/NRPS) gene cluster with 69 Kb. It was deduced from genome mining from the whole sequenciation of the genome of strain PHM005 composed by 20 ORF homologous to the described for pederin gene cluster. It contains genes encoding enzymes for the biosynthesis of pederin-like and onnamide-like compounds.

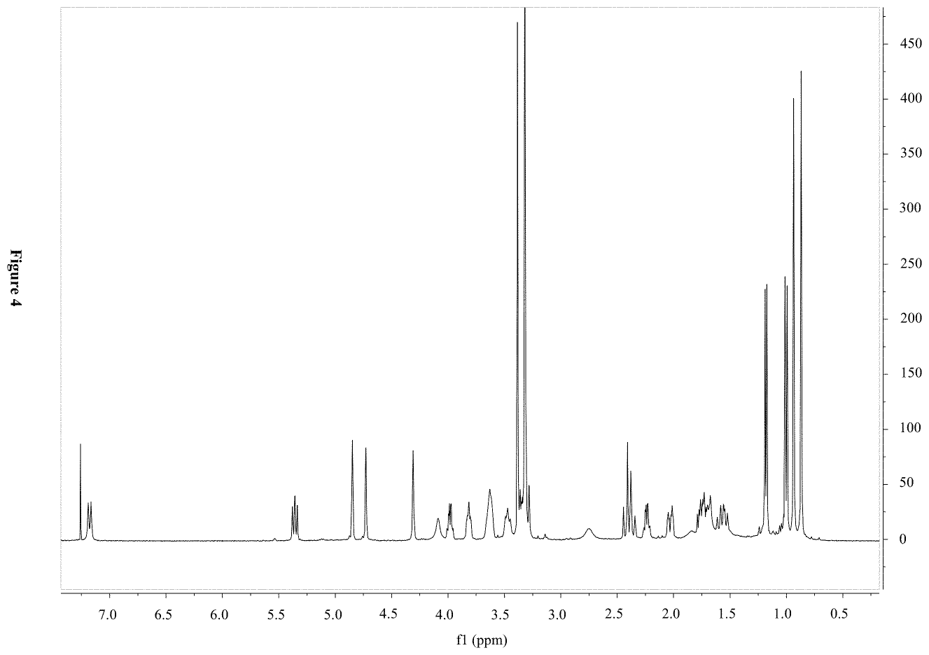

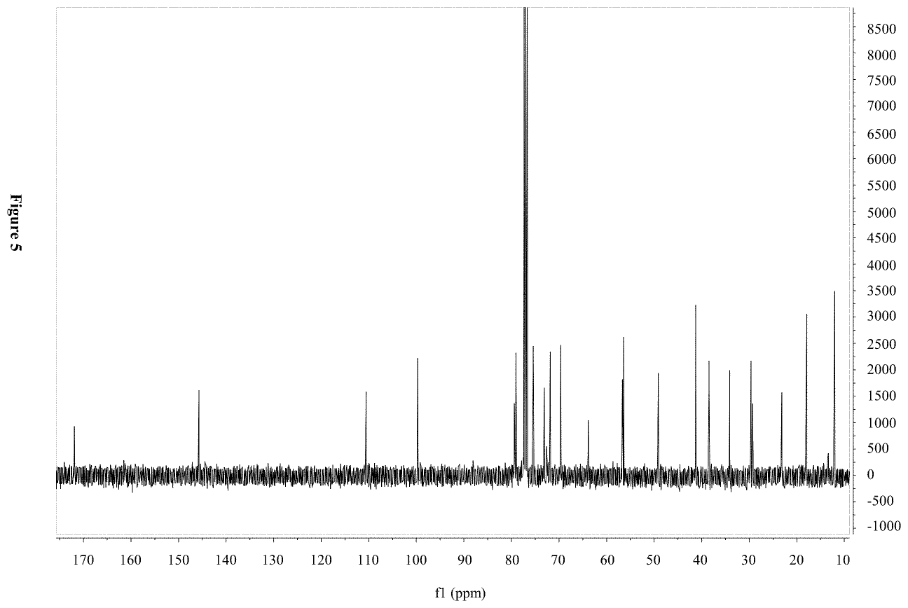

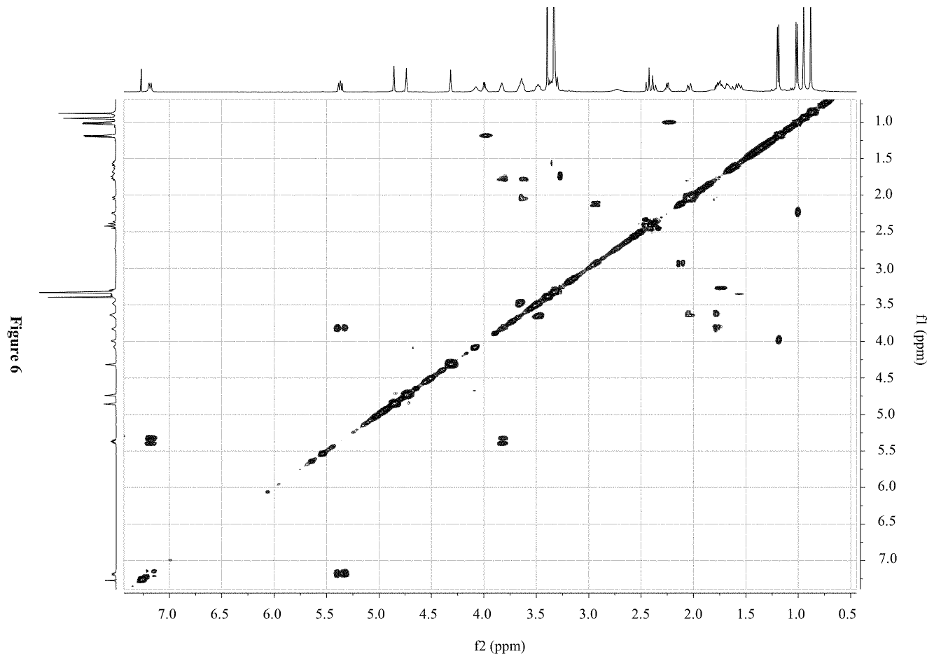

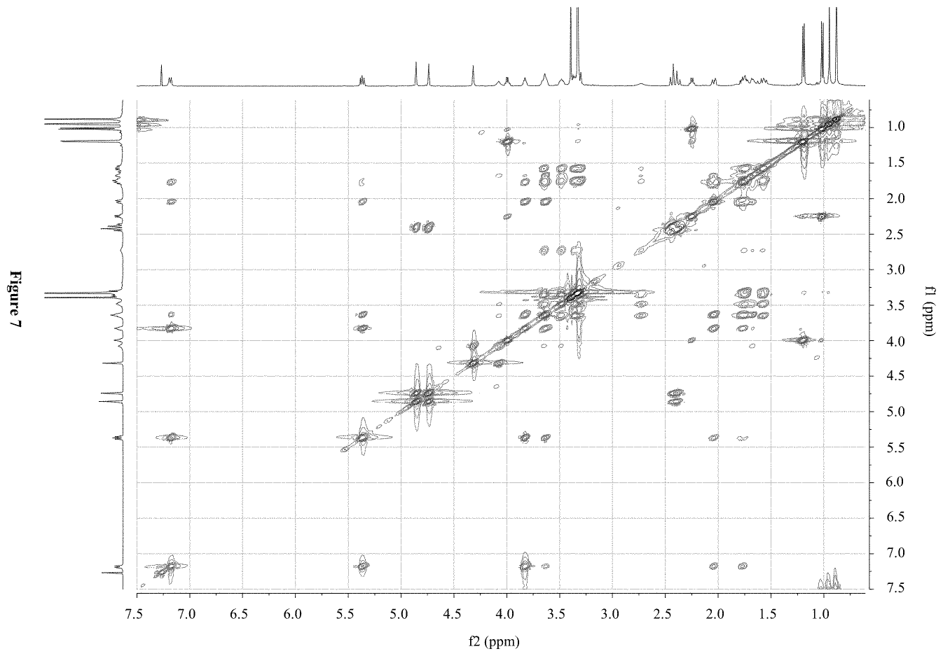

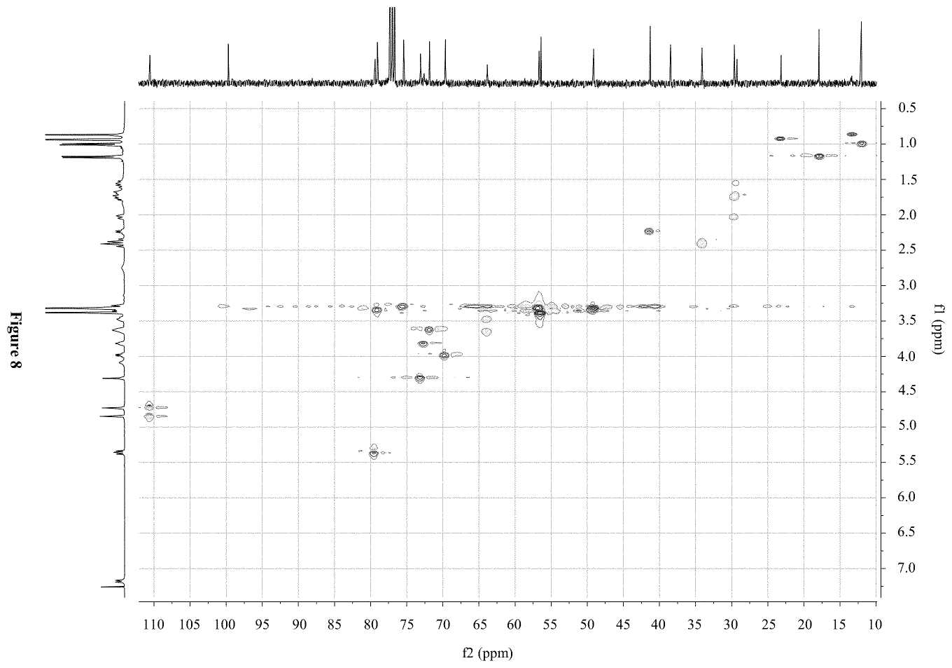

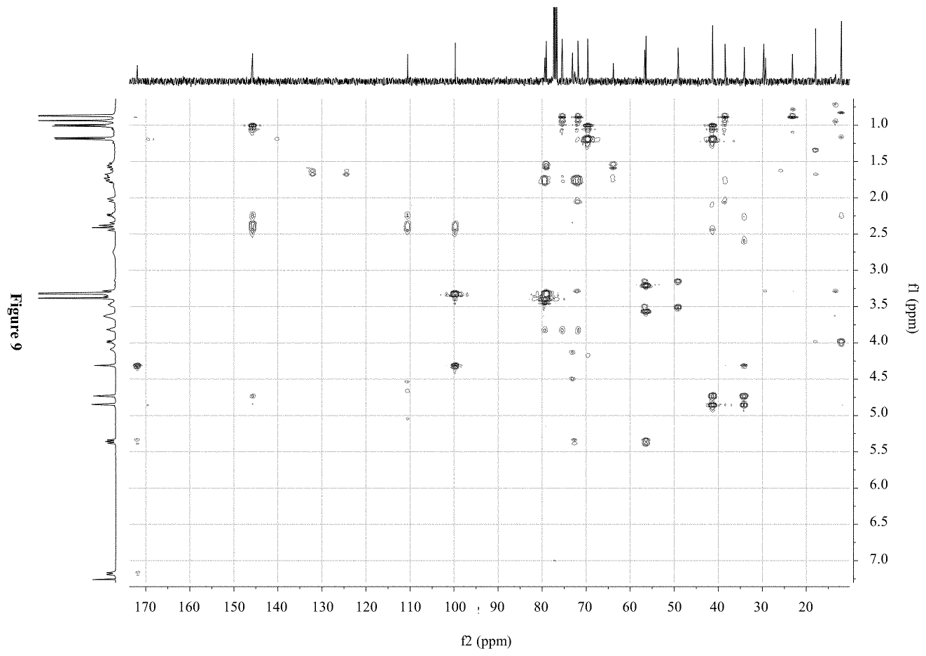

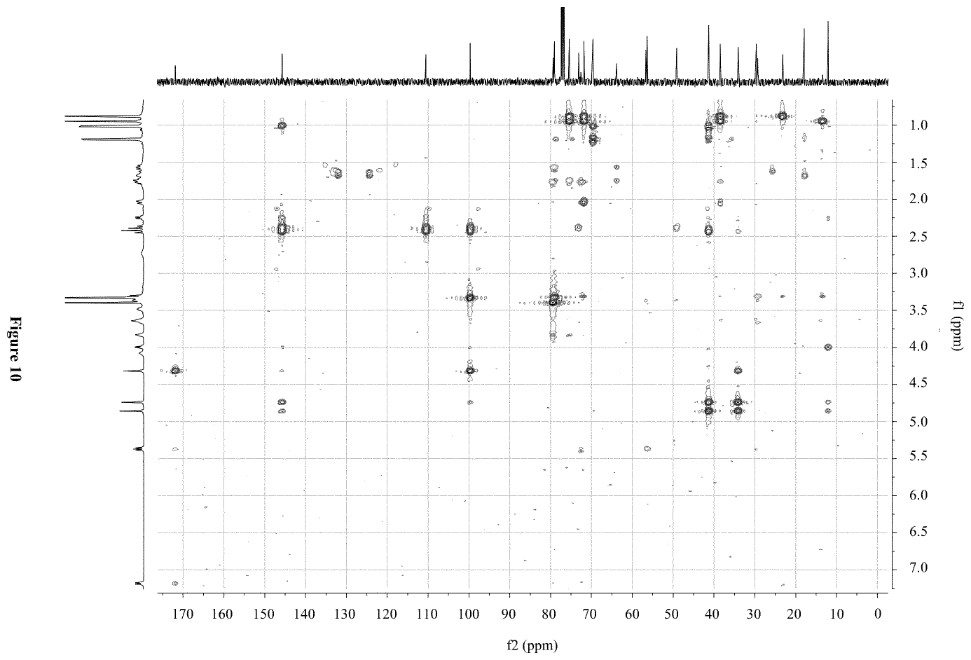

In a preferred embodiment, the isolated nucleic acid preferably comprises nucleic acid fragments forming individual units and/or modules of the Lab biosynthetic gene cluster as it is shown in more detail in FIG. 3. As depicted in FIG. 3, the Lab gene cluster contains the units lab706 to lab726.

In a particularly preferred embodiment, the isolated nucleic acid according to the eighth aspect of the present invention comprises:
a nucleotide sequence as shown in SEQ ID NO: 2; or
a nucleotide sequence which is the complement of SEQ ID NO: 2; or
a nucleotide sequence hybridising under highly stringent conditions to SEQ ID NO: 2; or to the complement thereof; or
a nucleotide sequence having at least 80% sequence identity with SEQ ID NO: 2 or with the complement thereof.

Particularly preferred nucleic acid fragments according to the nineth aspect of the present invention are nucleic acid fragments essentially comprising at least one of the genes lab708, lab709, lab710, lab721, lab722, lab723, lab724 and lab725. Further preferred are the nucleic acid fragments comprising one or more nucleotide sequences encoding the protein sequences as shown in SEQ ID NOs: 3 to 23. Also preferred parts are the corresponding parts of the nucleotide sequence SEQ ID NO: 2.

In another preferred embodiment particularly preferred fragments are those essentially consisting of lab719 and/or lab720. Further preferred is the nucleic acid fragment comprising the nucleotide sequence encoding the protein sequence as shown in SEQ ID NO: 16 and/or SEQ ID NO: 17. Also preferred are the corresponding parts of the nucleotide sequence SEQ ID NO: 2.

The annotation of the whole genome of PHM005 reveals a circular chromosome with a length of 6167 bp, 5651 coding sequences (CDS), 53 tRNA and 10 rRNA. 55% G+C.

Exploring the entire genome into a unique contig using software for prediction/identification of secondary metabolisms as antiSMASH V 3.0 (Weber and co-workers, Nucleic Acid Research, 2015 doi: 10. 1093/nar/gkv437) a 102 Kb of a large hybrid PKS/NRPS gene cluster is detected. Among the 317 ORF analyzed, 20 genes (69 Kb) shown homologies to pederin (ped) and onnamide (onn) sequences based on BLASTp against symbiont bacterium of Paedeus fascipens (GenBank AH013687.2) and bacterium symbiont of Theonella swinhoei (GenBank AY688304.1) as shown in more detail in Table 1.

TABLE 1
Homologies of the lab genes respect to ped (pederin) and onn (onnamide) genes.

| | | | Bacterium Symbiont | | Bacterium Symbiont | |
|---|---|---|---|---|---|---|
| | Prot. | | Paedeus fuscipens | | Theonella swinhoei | |
| lab | Size | Putative function in | (AH013687.2) | | (AY688304.1) | |
| gene | (aas) | Labrenzia sp. PHM005 | Gene | % H/Q* | Gene | % H/Q |
| 706 | 80 | Polyketide Biosynthesis Acyl | pedN | 47/87 | — | No |
| | | carrier protein (ACP) | | | | homology |
| 707 | 425 | Polyketide Biosynthesis | pedP | 61/99 | onnA | 60/99 |
| | | 3-hydroxy-3 methylglutaryl ACP | | | | |
| | | synthase (HMGS) | | | | |
| 708 | 1165 | Polyketide synthase | pedI | 42/93 | onnB | 39/98 |
| | | (GNAT-ACP-KS-DHt) | | | | |
| 709 | 3219 | TransAT PKS | pedI | 49/94 | onnB | 41/73 |
| | | (KR-cMT-ACP-KS-TransAT- | | | onnI | 45/73 |
| | | ECH-ACPb-ACPb-KS-KR) | | | | |
| 710 | 97 | Phosphopantetheine attached site | pedI | 46/90 | onnI | 34/73 |
| | | (ACP) | | | | |
| 711 | 373 | Monooxygenase (OX) | pedJ | 60/98 | onnC | 58/98 |
| 712 | 318 | Methyltransferase | pedA | 47/97 | onnG | 51/99 |
| | | (oMT) | | | onnD | 46/97 |
| 713 | 414 | Cytochrome P450 | | No | | No |
| | | | | homology | | homology |
| 714 | 447 | Malonyl CoA-ACP transacylase | pedB | 56/98 | | No |
| | | (or oxidorectase) | | | | homology |
| 715 | 337 | Malonyl CoA-ACP transcylase | pedC | 38/94 | | No |
| | | | | | | homology |
| 716 | 375 | Malonyl CoA-ACP transcylase | pedD | 51/95 | | No |
| | | | | | | homology |
| 717 | 253 | Enoyl transferase | pedL | 43/91 | | No |
| | | | | | | homology |
| 718 | 411 | Beta-ketocacyl-synthase | pedM | 30/81 | | No |
| | | | | | | homology |
| 719 | 2254 | Mixed TransAT PKS/NRPS | pedH | 42/99 | onnI | 35/84 |
| | | (ACP-KS-TransAT-DH-KR- | | | | |
| | | ACP-KS-DH-DH-ACP-KS- | | | | |
| | | TransAT-KR-ACP-KS- | | | | |
| | | TransAT-C-A-PCP-TE) | | | | |
| 720 | 437 | Oxidoreductase (Ox) | pedG | 73/94 | | No |
| | | | | | | homology |
| 721 | 1986 | TransAT-PKS | pedF | 40/99 | onnI | 30/82 |
| | | (PS-KR-ACP-KS-TransAT-KR- | | | | |
| | | KS-TransAT) | | | | |
| 722 | 1949 | TransAT Polyketide synthase | pedF | 44/97 | onnI | 36/86 |
| | | (Trans AT-KR-cMT-ACPb-KS- | | | onnB | 34/85 |
| | | TransAT-DH) | | | | |
| 723 | 875 | Polyketide synthase | pedF | 49/93 | onnB | 52/96 |
| | | (KR-ACP-KS) | | | onnI | 45/95 |
| 724 | 1986 | Mixed PKS/NRPS | pedF | 42/99 | onnI | 38/96 |
| | | (DHt-ACP-C-A(gly)-PCP-KS- | | | | |
| | | TransAT) | | | | |
| 725 | 377 | Polyketide synthase (KS) | pedF | 48/99 | onnI | 46/92 |
| | | | | | onnB | 41/88 |
| 726 | 278 | Methyltransferase (MT) | pedE | 51/98 | onnH | 43/99 |

*H: Homology in %.
Q: Query covered in %

The putative Lab gene cluster comprises a 69 Kb nucleic acid fragments forming individual units and/or modules similar to those described for pederin biosynthetic gene cluster as it is shown in more detail in FIG. 3.

The TransAT hybrid PKS/NRPS Lab gene cluster is mainly composed by one PKS (Composed by ORF lab708, lab709 and lab710) and two mixed PKS/NRPS systems (lab721, lab722, lab723, lab 724, lab 725 and lab 719) flanked by oxygenases, oxidoreductases and methylases in closed similar architecture to the described by J. Piel for ped genes. The predicted functions and the composition of the aminoacids of each ORF is detailed in Table 1.

The TransAT-PKS lab708, lab709, lab710 (4.481 amino acids) is composed by the modules GNAT-ACP-KS-DHt-KR-cMT-ACP-KS-TransAT-ECH-ACP-ACP-KS-KR-ACP) similar to the described for peril with homologies 42-49%. This biosynthetic gene cluster may be the responsible of the biosynthesis of the six membered ring bearing the exomethylene group of the pederin structure. Where the domains are GNAT: Gcn5-related-N-acetyltransferase; ACP: Acyl Carrier Protein; KS: Ketosynthase; DHt Dehydratase; KR: Ketoreductase; cMT: Methyltransferase; ECH Enoyl-CoA-hydratase o crotonase; TransAT: Trans Acyl Transferase).

The hybrid Trans-AT PKS/NRPS formed by lab721, lab722, lab723, lab724, lab725 (5.385 aa) is composed by 6 Kethosyntases and 1 NRPS with a clear adenylation for glycine. (PS-KR-ACP-KS-TransAT-KR-KS-TransAT-transAT-KR-cMT-ACP-KS-TransAT-DH-KR-ACP-KS-DHt-ACP-C-A (gly)-PCP-KS-TransAT-KS). With 40-49% homology to pedF but essentially the same functions and architecture of modules. Where the domains are C: nonribosomal peptide Condensation; A: nonribosomal peptide Adenylation; PCP: Thiolation and Peptide Carrier Protein.

According to a preferred embodiment of the nineth aspect, we have identified the lab719 PKS/NRPS system related to the biosynthesis of any onnamide-like compound from the Lab gene cluster. This putative new compound has not been identified in the fermentation broth of PHM005. It is possible that the product of the gen lab720, an oxidoreductase, will prevent the formation of the onnamide-like compound by cleaving the pederin structure before to add to the first domain ACP in lab719 or a final oxidative breakout is produced after its biosynthesis. The same doubt has been discussed by J. Piel in the WO 03/044186 A2. The genetic modification of the gene lab719 (homology to pedG) will solve this uncertainty.

This “silent” hybrid transAT PKS/NRPS gene, represented by lab719 (2.254 aa) is composed by 4 KS and 1 NRPS with uncertain adenylation domain, maybe for the incorporation of arg (as the case of onnamide), but asp, asn, glu and gln could be other possible alternatives as propodes by NRPSPredictor2 SVM algorithm. The composition of this ORF is (ACP-KS-TransAT-DH-KR-ACP-KS-DH-DH-ACP-KS-TransAT-KR-ACP-KS-TransAT-C-A-PCP-TE). Where TE: Thioesterase domain.

The single ORF in the lab region without sequence-homology to ped, onn or nsp (nosperin) islands is the lab713, putative for a cytochrome P450, maybe playing a role in oxygenation of polyketides, as described by J. Piel in the case of the ped islands. (J. Bacteriol. 2004. 186(5), 1280-1286) with similar function-assigned genes.

Particularly preferred modular enzymatic system according to the tenth aspect of the present invention comprises a protein sequence according to any of the sequences SEQ ID NO: 3 to SEQ ID NO: 23 or a protein sequence having at least 80% sequence identity with these sequences.

Particularly preferred host cells according to the twelfth aspect of the present invention are bacterial cells. More particularly preferred host cells are Pseudomonas, Acinetobacter, Bacillus, Streptomyces and E. coli.

The inventive modification on Lab biosynthetic gene cluster can be used in the preparation of a modified Lab biosynthetic gene cluster or in the preparation of pederin-like or onnamide-like compounds.

In a preferred embodiment according to the thirteenth aspect of the present invention the product of the lab719 is expressed.

### EXAMPLES

General Structure Elucidation Procedure. Optical rotations were determined using a Jasco P-1020 polarimeter. NMR spectra were obtained on a Varian “Unity 500” spectrometer at 500/125 MHz (¹H/¹³C) and on a Varian “Unity 400” spectrometer at 400/100 MHz (¹H/¹³C). Chemical shifts were reported in ppm using residual solvent peak for CDCl₃ (δ 7.26 ppm for ¹H and 77.0 ppm for ¹³C) as an internal reference. (+)ESIMS were recorded using an Agilent 1100 Series LC/MSD spectrometer. High Resolution Mass Spectroscopy (HRMS) was performed using an Agilent 6230 TOF LC/MS system and the ESI-MS technique.

### Example 1: Bacteria Isolation

The pederin-type producing bacteria, Labrenzia sp., PHM005 was isolated from a sediment sample collected at a depth of 18 m from a highly epiphytic and unidentified coral-sponge habitat off the coast of Kenya in 2005. Approximately 5 grams of sea gravel material was collected in a 50 ml Falcon tube containing sterile artificial sea water (ASW) and was maintained at 5° C. for 5 days before being processed. Once in the laboratory, the sample was homogenized and 100 μl of a 1:100 dilution with ASW spread directly on Petri dishes with a sea salt medium consisting of 27 g/L marine salts (Tropic Marin® PRO-REEF), 16 g/L agar and 0.2 mg/mL of cycloheximide After incubation for three weeks at 28° C., a slightly brown colony was picked and transferred to the same sea salt medium to confirm the purity and generate biomass for molecular characterization with one colony being inoculated on liquid marine broth for further conservation on 20% glycerol at -80° C. as a cell bank.

### Example 2: Electron Microscopy

Cells in the mid-exponential growth phase were adsorbed on 400-mesh carbon-collodion-coated grids for 2 min, negatively stained with 2% uranyl acetate, imaged with a Jeol JEM 1011 transmission electron microscope operated at 100 kV and photographed with a CCD Gatan Erlangshen ES1000W camera.

### Example 3: 16S rRNA Characterization

For DNA extraction the strain was grown in marine broth (DIFCO 1196) for 72 hours. Cells were recovered and lysed by boiling with 4% NP40 for 10 minutes. Cell debris was discarded by centrifugation. The 16S rDNA gene was amplified by the polymerase chain reaction using the bacterial primers Fl and R₅. The phylogenetic tree (FIG. 2) was generated by the Pairwise alignment based similarity coefficient and UPGMA for Cluster analysis using BioNumerics V7.5 (Applied Maths). The phylogenetic neighbors were identified and pairwise 16S rDNA gene sequence similarities calculated by comparison with the SILVA LTPs123 database.

### Example 4: Cultivation and Extraction

The strain clearly needs marine salt to grow. After culture, the whole broths were lyophilized and extracted with a mixture of organic solvents and a 0.5 mL sample of the crude extract dried and screened for cytotoxic activity. The best cytotoxic activity was achieved in the 16B/d medium at 120h. This medium consisted of 17.5 g/L of brewer's yeast (Sensient, G2025), 76 g/L mannitol, 7 g/L (NH4)₂SO₄, 13 g/L CaCO₃, 0.09 g/L FeCl₃ and 36 g/L marine salts (Tropic Marin® PRO-REEF). A 50 L scale-up of this bacterium in 16B/d medium was prepared in 200×2L Erlenmeyer flasks each with a working volume of 250 mL. The production flasks were inoculated with 2% of the bacteria grown during 72 h in marine broth (DIFCO 1196) from another highly grown pre-inoculum. The scale-up was incubated during 120h at 28° C. in a rotatory shaker at 220 rpm with 5 cm 4eccentricity. The culture was then centrifuged at 6.000 rpm during 20 minutes to give 45 L of aqueous supernatant which was extracted twice with EtOAc and the organic phase dried to give a crude extract (1.8 g).

### Example 5: Isolation of Compound 1

The extract was applied to a silica gel VFC (vacuum flash chromatography) system, using a stepwise gradient elution with n-hexane-EtOAc and EtOAc-MeOH mixtures to give eleven fractions. The active fractions were eluted with EtOAc and EtOAc-MeOH 9:1 (550.0 mg) and were subjected to preparative reversed-phase HPLC using a symmetry C₁₈ column (19×150 mm, 7 gm) and a linear gradient of H₂O/CH₃CN from 5% to 35% CH₃CN over 30 min at a flow rate of 13.5 mL/min, to afford a very active peak-fraction (77.0 mg) with a retention time of 24.5 min containing 1 based on the HPLC-MS chromatogram. This fraction was further purified by semipreparative HPLC on a XBridge C₁₈ column (10×250 mm, 5 μm) and isocratic elution with H₂O/CH₃CN (78:22) at a flow of 4 mL/min, to yield 24.5 mg of pure compound 1 with a retention time of 25.0 min at these HPLC conditions.

(1): colorless oil; [α]_{D}²⁰+82.4 (c=0.49; CHCl₃) and [α]_{D}²⁰+81.3 (c=0.36; MeOH); ¹H NMR (CDCl₃) δ 3.99 (1H, dq, J=6.6, 2.7 Hz, H-2), 2.25 (1H, dq, J=7.1, 2.7 Hz, H-3), 2.43 (1H, d, J=14.1 Hz, H-5a), 2.36 (1H, dt, J=14.1, 2.3 Hz, H-5b), 4.31 (1H, s, H-7), 7.18 (1H, d, J=9.8 Hz, NH), 5.37 (1H, dd, J=9.8, 7.8 Hz, H-10), 3.83 (1H, dt, J=7.8, 2.7 Hz, H-11), 2.04 (1H, dt, J=13.5, 3.6 Hz, H-12a), 1.75 (1H, m, H-12b), 3.64 (1H, m, H-13), 3.31 (1H, m, H-15), 1.75 (1H, m, H-16a), 1.57 (1H, dd, J=14.3, 9.7 Hz, H-16b), 3.36 (1H, m, H-17), 3.65 (1H, m, H-18a), 3.48 (1H, m, H-18b), 1.19 (3H, d, J=6.6 Hz, H-19), 1.01 (3H, d, J=7.1 Hz, H-20), 4.85 (1H, t, J=2.3 Hz, H-21a), 4.73 (1H, t, J=2.3 Hz, H-21b), 0.95 (3H, s, C-22), 0.88 (3H, s, C-23), 3.32 (3H, s, MeO-6), 3.38 (3H, s, MeO-10), 3.32 (3H, s, MeO-17); ¹³C NMR (CDCl₃) δ 69.6 (d, C-2), 41.3 (d, C-3), 145.7 (s, C-4), 34.1 (t, C-5), 99.7 (s, C-6), 73.1 (d, C-7), 171.9 (s, C-8), 79.4 (d, C-10), 72.6 (d, C-11), 29.6 (t, C-12), 71.8 (d, C-13), 38.4 (s, C-14), 75.4 (d, C-15), 29.2 (t, C-16), 79.0 (d, C-17), 63.8 (t, C-18), 17.9 (q, C-19), 12.0 (q, C-20), 110.5 (t, C-21), 23.1 (s, C-22), 13.5 (s, C-23), 49.1 (q, MeO-6), 56.4 (q, MeO-10), 56.6 (q, MeO-17); (+)-ESIMS m/z 512.3 [M+Na]⁺; (30 )-HRES-TOFMS m/z 512.2873 [M+Na]⁺ (calcd. for C₂₄H₄₃NO₉Na, 512.2830).

The relative stereochemistry of compound 1 was established as

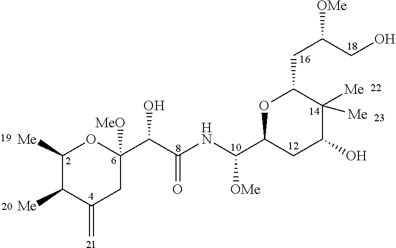

on the basis of ROESY data and analysis of coupling constants. The optical rotation of compound 1 ([α]_{D}²⁰+82.4, c=0.49; CHCl₃ and [α]_{D}²⁰+81.3, c=0.36; MeOH) showed the same sign as pederin ([α]_{D}²⁰+86.8, c=1.00; CHCl₃). The absolute stereochemistry of pederin has been established by X-ray crystallographic study (Simpson, J. S. et. al. J. Nat. Prod. 2000, 63, 704-706) and stereoselective synthesis (Matsuda, F., et. al. Tetrahedron 1988, 44, 7063-7080). Therefore, we tentatively propose the absolute configuration of compound 1 to be the same as pederin and other reported analogous compounds (Wan, S. et. al. J. Am. Chem. Soc. 2011, 133, 16668-16679).

### Example 6. Isolation of Compound 2

Compound 2 was isolated from the whole broth crude extract (9.5 g) of the fermentation broth (15 L) of the marine derived strain PHM005. The extract was applied to a silica gel VFC (vacuum flash chromatography) system, using a stepwise gradient elution with n-hexane-EtOAc and EtOAc-MeOH mixtures to give seven fractions. The active fraction containing compound 2 was eluted with EtOAc-MeOH 4:1 (659.0 mg) and were subjected to semipreparative reversed-phase HPLC equipped with a Symmetry C₁₈ column (7.8×150 mm, 51.1m) using a linear gradient of H₂O/CH₃CN from 5% to 60% of CH₃CN in 25 min at a flow rate of 3.0 mL/min, to afford a very active time-fraction between 25-30 min (28.0 mg) containing compound 2 based on HPLC-MS chromatogram. This fraction was again purified by semipreparative HPLC on a Symmetry C18 column (7.8×150 mm, 5 μm), using a linear gradient of H₂O/CH₃CN from 20% to 30% of CH₃CN in 20 min at a flow rate of 2.5 mL/min, to yield 2.6 mg of pure compound 2 with a retention time of 11.5 min at these HPLC conditions.

2: colorless oil; [α]_{D}²⁰+64.5 (c=0.16; CHCl₃); ¹H NMR (CDCl₃) δ 3.97 (1H, dq, J=6.6, 2.6 Hz, H-2), 2.25 (1H, dq, J=7.1, 2.6 Hz, H-3),), 2.50 (1H, dt, J=14.2, 1.45 Hz, H-5a), 2.45 (1H, d, J=14.1 Hz, H-5b), 4.32 (1H, s, H-7), 7.17 (1H, d, J=9.9 Hz, NH), 5.44 (1H, dd, J=9.9, 7.5 Hz, H-10), 3.95 (1H, m, H-11), 2.05 (1H, dt, J=13.5, 4.0 Hz, H-12a), 1.75 (1H, m, H-12b), 3.66 (1H, m, H-13), 3.58 (1H, m, H-15), 1.80 (1H, m, H-16a), 1.55 (1H, m, H-16b), 3.80 (1H, m, H-17), 3.57 (1H, m, H-18), 3.44 (1H, dd, J=11.5, 6.5 Hz, H-18), 1.19 (3H, d, J=6.6 Hz, H-19), 1.01 (3H, d, J=7.1 Hz, H-20), 4.85 (1H, t, J=1.45 Hz, H-21a), 4.75 (1H, t, J=1.45 Hz, H-21b), 0.96 (3H, s, C₂₂), 0.89 (3H, s, C-23), 3.34 (3H, s, MeO-6), 3.41 (3H, s, MeO-10); ¹³C NMR (CDCl₃) δ 69.6 (d, C-2), 41.3 (d, C-3), 146.1 (s, C-4), 34.2 (t, C-5), 99.6 (s, C-6), 74.5 (d, C-7), 171.9 (s, C-8), 79.3 (d, C-10), 72.2 (d, C-11), 29.8 (t, C-12), 71.6 (d, C-13), 38.4 (s, C-14), 80.9 (d, C-15), 31.4 (t, C-16), 72.8 (d, C-17), 66.6 (t, C-18), 17.8 (q, C-19), 11.9 (q, C-20), 110.2 (t, C-21), 23.4 (s, C-22), 14.3 (s, C-23), 49.6 (q, MeO-6), 56.3 (q, MeO-10); (+)-ESIMS m/z 498.4 [M+Na]⁺; (+)-HRES-TOFMS m/z 498.2713 [M+Na]⁺ (calcd. for C₂₃H₄₁NO₉Na, 498.2674).

The relative stereochemistry of compound 2 was assigned as

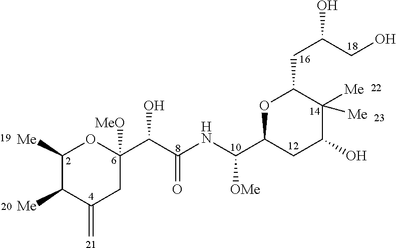

on the basis of an analysis of coupling constants. The optical rotation of compound 2 ([α]_{D}²⁰+64.5, c=0.16; CHCl₃) showed the same sign as pederin ([α]_{D}²⁰+86.8, c=1.00; CHCl₃). Therefore, we tentatively propose the absolute configuration of compound 2 to be the same as pederin and other reported analogous compounds (Wan, S. et. al. J. Am. Chem. Soc. 2011, 133, 16668-16679).

### Example 7. Synthesis of Compound 3

To a solution of 1 (2.5 mg, 5.1 μmol) in dry DCM (2 mL) under a nitrogen atmosphere, were added pyridine (10 μL, 124 μmol), DMAP (catalytic amount) and Ac₂O (2.9 μL, 31 mmol). The reaction was allowed to stand at room temperature overnight. The mixture was concentrated under vacuum and purified via flash column chromatography on silica gel (n-hexane/EtOAc 1:1) to afford 3 (3 mg, 95%) as a white solid.

3: ¹H NMR (CDCl₃) δ 3.96 (1H, dq, J=6.6, 2.6 Hz, H-2), 2.24 (1H, dq, J=7.0, 2.6 Hz, H-3), 2.62 (1H, dt, J=14.5, 2.2 Hz, H-5a), 2.37 (1H, d, J=14.5 Hz, H-5b), 5.25 (1H, s, H-7), 6.62 (1H, d, J=9.6 Hz, NH), 5.27 (1H, dd, J=9.6, 4.1Hz, H-10), 3.91(1H, dt, J=6.3, 4.6, Hz, H-11), 2.02 (1H, m, H-12a), 1.66 (1H, m, H-12b), 4.91 (1H, dd, J=4.7, 4.1Hz, H-13), 3.55 (1H, m, H-15), 2.02 (1H, m, H-16a), 1.67 (1H, m, H-16b), 3.60 (1H, dd, J=11.3, 2.2 Hz, H-17), 4.32 (1H, dd, J=12.1, 2.6 Hz, H-18a), 4.12 (1H, m, H-18b), 1.15 (3H, d, J=6.6 Hz, H-19), 0.97 (3H, d, J=7.0 Hz, H-20), 4.86 (1H, t, J=2.0 Hz, H-2a), 4.76 (1H, t, J=2.0 Hz, H-21b), 0.97 (3H, s, C₂₂), 0.89 (3H, s, C-23), 3.21 (3H, s, MeO-6), 3.39 (3H, s, MeO-10), 3.38 (3H, s, MeO-17), 2.20 (3H, s, OCOMe-7), 2.08 (3H, s, OCOMe-13), 2.10 (3H, s, OCOMe-18); 13C NMR (CDCl₃) δ 69.6 (d, C-2), 41.3 (d, C-3), 145.5 (s, C-4), 33.8 (t, C-5), 99.1 (s, C-6), 72.1 (d, C-7), 167.4 (s, C-8), 81.8 (d, C-10), 70.0 (d, C-11), 26.7 (t, C-12), 74.2 (d, C-13), 36.7 (s, C-14), 76.5 (d, C-15), 29.3 (t, C-16), 76.4 (d, C-17), 64.0 (t, C-18), 17.9 (q, C-19), 12.0 (q, C-20), 110.4 (t, C-21), 24.7 (s, C-22), 17.2 (s, C-23), 48.4 (q, MeO-6), 56.3 (q, MeO-10), 57.0 (q, MeO-17), 20.7 (q, OCOMe-7), 169.8 (s, OCOMe-7), 21.2 (q, OCOMe-13), 170.3 (s, OCOMe-13), 20.9 (q, OCOMe-18), 170.0 (s, OCOMe-18),; (+)-ESIMS m/z 638.3 [M+Na]⁺.

The relative stereochemistry of compound 3 was established as

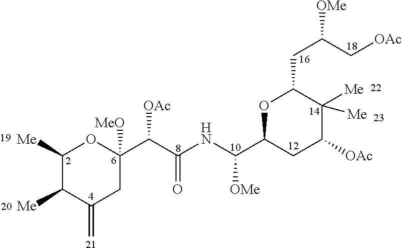

by analogy with its precursor, compound 1.

### Example 8. In Vitro Bioassays for the Detection of Antitumor Activity

The aim of this assay is to evaluate the in vitro cytostatic (ability to delay or arrest tumor cell growth) or cytotoxic (ability to kill tumor cells) activity of the samples being tested.

### Cell Lines

| Name | No ATCC | Species | Tissue | Characteristics |
|---|---|---|---|---|
| A549 | CCL-185 | human | lung | lung carcinoma |
| | | | | (NSCLC) |
| HT29 | HTB-38 | human | colon | colorectal |
| | | | | adeno- |
| | | | | carcinoma |
| MDA-MB-231 | HTB-26 | human | breast | breast |
| | | | | adeno- |
| | | | | carcinoma |
| PSN1 | CRM-CRL-3211 | human | pancreas | pancreas |
| | | | | adeno- |
| | | | | carcinoma |

### Evaluation of Cytotoxic Activity Using the SBR Colorimetric Assay

A colorimetric assay, using sulforhodamine B (SRB) reaction has been adapted to provide a quantitative measurement of cell growth and viability (following the technique described by Skehan et al. J. Natl. Cancer Inst. 1990, 82, 1107-1112).

This form of assay employs 96-well cell culture microplates following the standards of the American National Standards Institute and the Society for Laboratory Automation and Screening (ANSI SLAS 1-2004 (R₂₀₁₂) 10/12/2011). All the cell lines used in this study were obtained from the American Type Culture Collection (ATCC) and derive from different types of human cancer.

Cells were maintained in Dulbecco's Modified Eagle Medium (DMEM) supplemented with 10% Fetal Bovine Serum (FBS), 2mM L-glutamine, 100 U/mL penicillin and 100 U/mL streptomycin at 37 ° C., 5% CO2 and 98% humidity. For the experiments, cells were harvested from subconfluent cultures using trypsinization and resuspended in fresh medium before counting and plating.

Cells were seeded in 96 well microtiter plates, at 5000 cells per well in aliquots of 150 μL and allowed to attach to the plate surface for 18 hours (overnight) in drug free medium. After that, one control (untreated) plate of each cell line was fixed (as described below) and used for time zero reference value. Culture plates were then treated with test compounds (50 μL aliquots of 4× stock solutions in complete culture medium plus 4% DMSO) using ten 2/5 serial dilutions (concentrations ranging from 10 to 0.003 μg/mL) and triplicate cultures (1% final concentration in DMSO). After 72 hours treatment, the antitumor effect was measured by using the SRB methodology: Briefly, cells were washed twice with PBS, fixed for 15 min in 1% glutaraldehyde solution at room temperature, rinsed twice in PBS, and stained in 0.4% SRB solution for 30 min at room temperature. Cells were then rinsed several times with 1% acetic acid solution and air-dried at room temperature. SRB was then extracted in 10 mM trizma base solution and the absorbance measured in an automated spectrophotometric plate reader at 490 nm. Effects on cell growth and survival were estimated by applying the NCI algorithm (Boyd M R and Paull K D. Drug Dev. Res. 1995, 34, 91-104).

The values obtained in triplicate cultures were fitted to a four-parameter logistic curve by nonlinear regression analysis. Three reference parameters were calculated (according to the NCI algorithm) by automatic interpolation of the curves obtained from such fitting: GI₅₀=compound concentration that produces 50% cell growth inhibition, as compared to control cultures; TGI=total cell growth inhibition (cytostatic effect), as compared to control cultures, and LC₅₀=compound concentration that produces 50% net cell killing cytotoxic effect).

TABLE 2
illustrates data on the biological activity
of compounds of the present invention.

| | Biological activity (M) | | | | |
|---|---|---|---|---|---|
| Com- | | Cell Line | | | |
| pound | | A549 | HT29 | MDA-MB-231 | PSN-1 |
| 1 | GL₅₀ | 2.04E−09 | 2.86E−09 | 2.66E−09 | 2.66E−09 |
| | TGI | 7.97E−09 | 8.99E−09 | 5.31E−09 | 5.72E−09 |
| | LC₅₀ | 3.68E−08 | >2.04E−07 | 1.08E−08 | 1.94E−08 |
| 2 | GI₅₀ | 7.15E−09 | 8.83E−09 | 8.20E−09 | 8.62E−09 |
| | TGI | 2.52E−08 | 4.42E−08 | 1.56E−08 | 1.91E−08 |
| | LC₅₀ | 1.22E−07 | >2.10E−06 | 3.15E−08 | 7.78E−08 |
| 3 | GI₅₀ | 1.15E−07 | 1.62E−07 | 3.09E−07 | 1.62E−07 |
| | TGI | 8.77E−07 | 9.26E−07 | 2.44E−06 | 6.66E−07 |
| | LC₅₀ | 8.61E−06 | >1.62E−05 | >1.62E−05 | 3.90E−06 |

1. A compound of general formula I or a pharmaceutically acceptable salt, tautomer, or stereoisomer thereof. 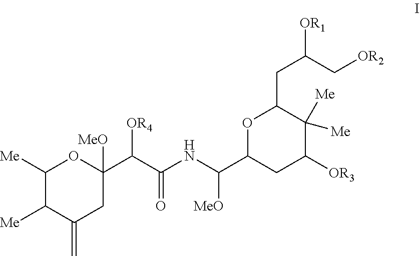
wherein:
R₁, R₂, and R₃ are each independently selected from hydrogen, substituted or unsubstituted C₁-C₁₂ alkyl, substituted or unsubstituted C₂-C₁₂ alkenyl, substituted or unsubstituted C₂-C₁₂ alkynyl, —C(═O)Rₐ, —C(═O)OR_{b} and —(C═O)NR_{c}R_{d};
R₄ is selected from hydrogen, —C(═O)Rₐ, —C(═O)OR_{b}, and —C(═O)NR_{c}R_{d};
Rₐ is selected from hydrogen, substituted or unsubstituted C₁-C₁₂ alkyl, substituted or unsubstituted C₂-C₁₂ alkenyl, substituted or unsubstituted C₂-C₁₂ alkynyl, aryl, and heterocyclyl;
R_{b} is selected from substituted or unsubstituted C₁-C₁₂ alkyl, substituted or unsubstituted C₂-C₁₂ alkenyl, substituted or unsubstituted C₂-C₁₂ alkynyl, aryl, and heterocyclyl;
R_{c} and R_{d} are independently selected from hydrogen, substituted or unsubstituted C₁-C₁₂ alkyl, substituted or unsubstituted C₂-C₁₂ alkenyl, substituted or unsubstituted C₂-C₁₂ alkynyl, aryl and heterocyclyl;
with the proviso that R₁ and R₂ are not simultaneously methyl.

2. The compound according to claim 1, also having general formula III or a pharmaceutically acceptable salt, tautomer or stereoisomer thereof. 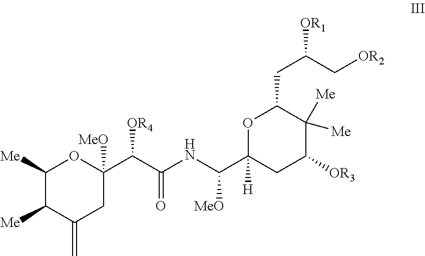
wherein R₁, R₂, R₃ and R₄ are as defined for formula I in claim 1.

3. The compound according to claim 1 or 2, wherein R₁ is selected from hydrogen and substituted or unsubstituted C₁-C₆ alkyl.

4. The compound according to claim 3, wherein R₁ is selected from hydrogen and methyl.

5. The compound according to claim 1, wherein R₂ is selected from hydrogen and —C(═O)Rₐ where Rₐ is selected from substituted or unsubstituted C₁-C₆ alkyl.

6. The compound according to claim 5, wherein R₂ is selected from hydrogen and acetyl.

7. The compound according to claim 1, wherein R₃ and R₄ are independently selected from hydrogen and —C(═O)Rₐ, wherein Rₐ at each occurrence is independently selected from substituted or unsubstituted C₁-C₆ alkyl.

8. The compound according to claim 7 wherein R₃ and R₄ are independently selected from hydrogen and acetyl.

9. The compound according to claim 1 of formula: 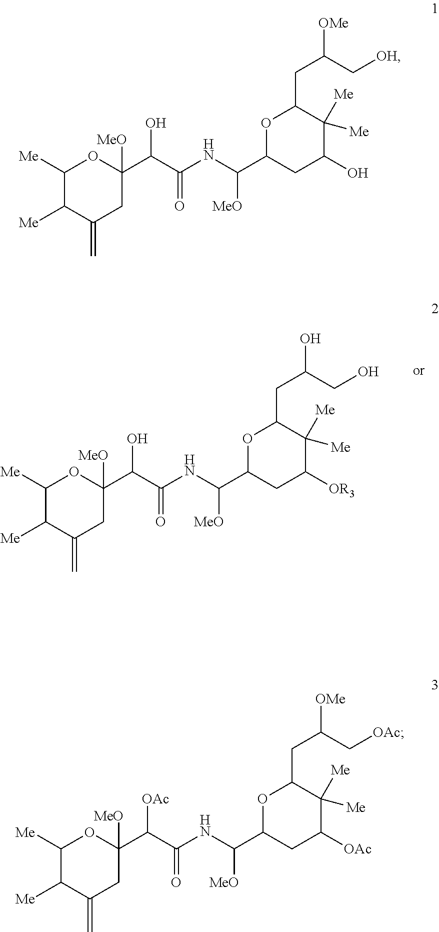
or
a pharmaceutically acceptable salt, tautomer or stereoisomer thereof.

10. The compound according to claim 9 of formula: 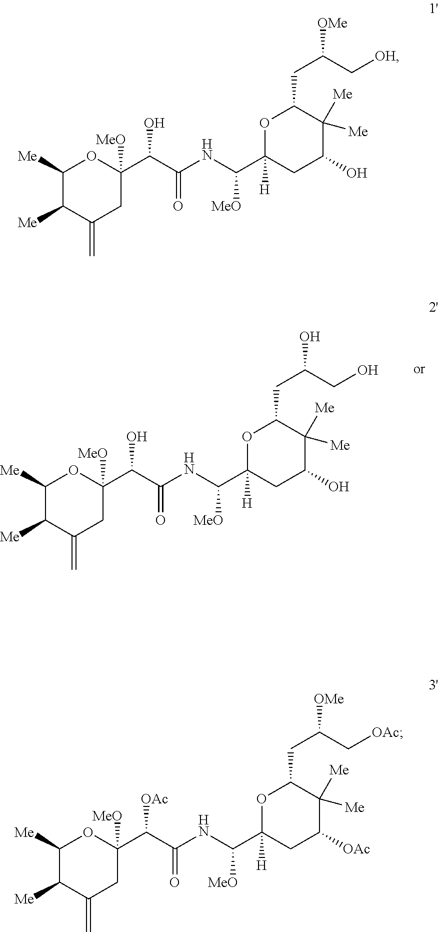
or
a pharmaceutically acceptable salt, tautomer or stereoisomer thereof.

11. A pharmaceutical composition comprising a compound as defined in claim 1, or a pharmaceutically acceptable salt, tautomer or stereoisomer thereof, and a pharmaceutically acceptable carrier or diluent.

12. A compound as defined in claim 1, or a pharmaceutically acceptable salt, tautomer or stereoisomer thereof, or a pharmaceutical composition comprising the compound, for use as a medicament.

13. The compound or composition according to claim 12, for use as a medicament for the treatment of cancer.

14. Use of the compound of claim 1, or pharmaceutically acceptable salts, tautomers, or stereoisomers thereof, in the preparation of a medicament for the treatment of cancer.

15. A method of treating a patient, notably a human, affected by cancer, which comprises administering to the affected individual in need thereof a therapeutically effective amount of the compound as defined in claim 1, or a pharmaceutically acceptable salt, tautomer, or stereoisomer thereof.

16. A process for obtaining a compound of formula II or a pharmaceutically acceptable salt, tautomer, or stereoisomer thereof. 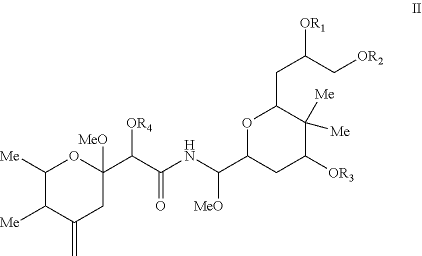
wherein
R₁, R₂, and R₃ are each independently selected from hydrogen, substituted or unsubstituted C₁-C₁₂ alkyl, substituted or unsubstituted C₂-C₁₂ alkenyl, substituted or unsubstituted C₂-C₁₂ alkynyl, —C(═O)Rₐ, —C(═O)OR_{b} and —(C═O)NR_{c}R_{d};
R₄ is selected from hydrogen, —C(═O)Rₐ, —C(═O)OR_{b}, and —C(═O)NR_{c}R_{d};
Rₐ is selected from hydrogen, substituted or unsubstituted C₁-C₁₂ alkyl, substituted or unsubstituted C₂-C₁₂ alkenyl, substituted or unsubstituted C₂-C₁₂ alkynyl, aryl, and heterocyclyl;
R_{b} is selected from substituted or unsubstituted C₁-C₁₂ alkyl, substituted or unsubstituted C₂-C₁₂ alkenyl, substituted or unsubstituted C₂-C₁₂ alkynyl, aryl, and heterocyclyl;
R_{c} and R_{d} are independently selected from hydrogen, substituted or unsubstituted C₁-C₁₂ alkyl, substituted or unsubstituted C₂-C₁₂ alkenyl, substituted or unsubstituted C₂-C₁₂ alkynyl, aryl and heterocyclyl;
the process comprising the steps of:
culturing the wild type marine bacterial strain PHM005 or their mutants under suitable conditions to produce compounds 1 and/or 2 of formula: 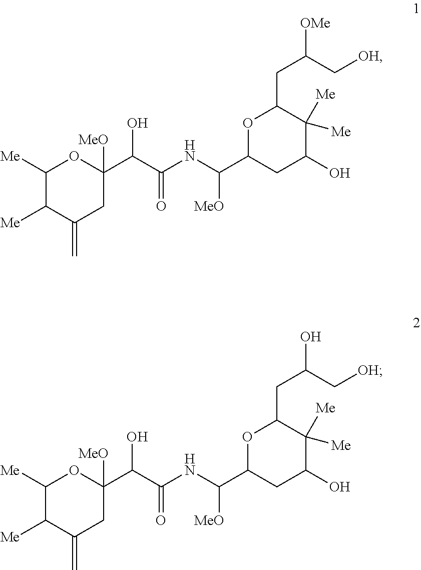 [SMILES: C=C1CC(OC)(C(O)C(=O)NC(OC)C2CC(O)C(C)(C)C(CC(CO)OC)O2)OC(C)C1C ; C=C1CC(OC)(C(O)C(=O)NC(OC)C2CC(O)C(C)(C)C(CC(O)CO)O2)OC(C)C1C]
isolating compounds 1 or 2; and, if needed,
derivatizing compounds 1 or 2.

17. The process according to claim 16, wherein the compound of formula II has also formula IV 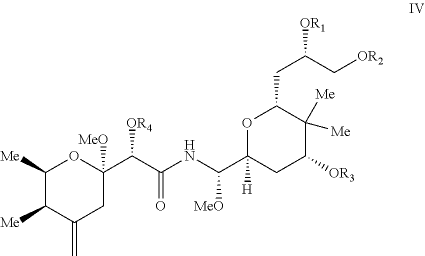
or a pharmaceutically acceptable salt, tautomer or stereoisomer thereof;
wherein R₁, R₂, R₃, and R₄ are as defined for formula II in claim 16.

18. Biologically pure strain PHM005, deposited under the accession number CECT-9225 in the Colección Española de Cultivos Tipo at the University of Valencia, Spain.

19. An isolated nucleic acid comprising the Lab biosynthetic gene cluster or being complementary to a sequence comprising the Lab biosynthetic gene cluster which is derived from Labrenzia sp. and in particular from strain PHM005.

20. The isolated nucleotide sequence according to claim 19 comprising:
a nucleotide sequence as shown in SEQ ID NO: 2; or
a nucleotide sequence which is the complement of SEQ ID NO: 2; or
a nucleotide sequence hybridising under highly stringent conditions to SEQ ID NO: 2 or to the complement thereof; or
a nucleotide sequence having at least 80% sequence identity with SEQ ID NO: 2 or with the complement thereof.

21. An isolated nucleic acid comprising nucleic acid fragments forming individual units and/or modules of the Lab biosynthetic gene cluster as shown in FIG. 3.

22. A modular enzymatic system encoded by a nucleic acid sequence as defined in any of claims 19 to 21.

23. A modular enzymatic system according to claim 22 comprising one or more of a protein sequence selected from the group formed by the sequences SEQ ID NO: 3 to SEQ ID NO: 23 or a protein sequence having at least 80% sequence identity with these sequences.

24. A modular enzymatic system according to claim 22 having functional activity in the synthesis of pederin-like or onnamide-like compounds and/or a polyketide moiety and/or a nonribosomal peptide moiety.

25. A vector comprising a nucleic acid consisting essentially of the Lab biosynthetic gene cluster derived from Labrenzia sp. and in particular from strain PHM005.

26. A vector comprising a nucleic acid sequence according to any of claims 19 to 21.

27. A recombinant host cell or a transgenic organism comprising a nucleic acid according to any of claims 19 to 21 or containing a vector comprising the nucleic acid.

28. A recombinant host cell according to claim 27 which is a bacterial cell and in particular is a Pseudomonas, Acinetobacter, Bacillus, Streptomyces, or E. coli cell.

29. A method for producing pederin-like or onnamide-like compounds using a mutant of PHM005 or a recombinant host cell according to claim 27 comprising the steps of:
culturing the mutant of PHM005 or the recombinant host cell or the transgenic organism under conditions to express the Lab biosynthetic gene cluster; and
isolating the produced pederin-like or onnamide-like compounds.

30. The method according to claim 29 wherein the product of the lab719 is expressed to provide an onnamide-like compound.

31. Use of a nucleic acid according to any of claims 19 to 21 in the preparation of a modified Lab biosynthetic gene cluster.

32. Use of a nucleic acid according to any of claims 19 to 21 in the preparation of a pederin-like compound.
